SEQUENCE LISTING (194 sequences)

SEQ ID NO: 1 - protein, 449 aa, Synthetic construct
EVQLLESGGGLVQPGGSLRLSCAASGFTFSSYYMSWVREAPGKGLEWVSTISESGDSTYYADSVKGRFTISRDNSKNTLYLQMNSLRAEDTAVYYCAKSGPGLRQVGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDEKVEPDSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPG

SEQ ID NO: 2 - protein, 214 aa, Synthetic construct
DIQMTQSPSSLSASVGDRVTITCRASQSISSYLNWYQKKPGKAPKLLIYAASSLQSGVPSRFSGSGSGTDFTLTISSLQPEDFATYYCQQVYSTPFTFGQGTKLEIKRTVAAPSVFIFPPSKKQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 446 aa, Synthetic construct
EVQLLESGGGLVQPGGSLRLSCAASGFTFSVYDMSWVRKAPGKRLEWVSTISSGGSFTHYPDSVKGRFTISRDNSKNTLYLQMNSLRAEDTAVYYCVRRSLDWYFDVWGAGTTVTVSSASTKGPSVFPLAPSSKSTSGGTAFLGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 4 - protein, 216 aa, Synthetic construct
EIVLTQSPGTLSLSPGERATLSCRVSSSIQSSNLHWYQEKPGQSPRPLIYGTSNLASGVPVRFSGSGSGTDYTLTISRLEPEDFAVYYCQQWSSYPLYTFGGGTKVEIKRTVAAPSVAIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 449 aa, Synthetic construct; identical to SEQ ID NO: 1

SEQ ID NO: 6 - protein, 214 aa, Synthetic construct; identical to SEQ ID NO: 2

SEQ ID NO: 7 - protein, 446 aa, Synthetic construct; identical to SEQ ID NO: 3

SEQ ID NO: 8 - protein, 216 aa, Synthetic construct
EIVLTQSPGTLSLSPGERATLSCRVSSSIQSSNLHWYQEKPGQSPRPLIYGTSNLASGVPVRFSGSGSGTDYTLTISRLEPEDFAVYYCQQWSSYPLYTFGGGTKVEIKRTVAAPSVFIAPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 449 aa, Synthetic construct; identical to SEQ ID NO: 1

SEQ ID NO: 10 - protein, 214 aa, Synthetic construct; identical to SEQ ID NO: 2

SEQ ID NO: 11 - protein, 446 aa, Synthetic construct
EVQLLESGGGLVQPGGSLRLSCAASGFTFSVYDMSWVRKAPGKRLEWVSTISSGGSFTHYPDSVKGRFTISRDNSKNTLYLQMNSLRAEDTAVYYCVRRSLDWYFDVWGAGTTVTVSSASTKGPSVFPLAPSSKSTSGGTAALGCLVSDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 12 - protein, 216 aa, Synthetic construct
EIVLTQSPGTLSLSPGERATLSCRVSSSIQSSNLHWYQEKPGQSPRPLIYGTSNLASGVPVRFSGSGSGTDYTLTISRLEPEDFAVYYCQQWSSYPLYTFGGGTKVEIKRTVAAPSVFIFPPSDEQLKSGTAKVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 216 aa, Synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 14 - protein, 216 aa, Synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 216 aa, Synthetic construct; identical to SEQ ID NO: 13

SEQ ID NO: 16 - protein, 216 aa, Synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 216 aa, Synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 18 - protein, 216 aa, Synthetic construct; identical to SEQ ID NO: 16

SEQ ID NO: 19 - protein, 216 aa, Synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 20 - protein, 216 aa, Synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 21 - protein, 216 aa, Synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 22 - protein, 216 aa, Synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 216 aa, Synthetic construct
APPVAGPSVFLFPPKPKDTLMISRTPEVTCVVVDVKHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 24 - protein, 216 aa, Synthetic construct
APPVAGPSVFLFPPKPKDTLMISRTPEVTCVVVDVKHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 216 aa, Synthetic construct
APPVAGPSVFLFPPKPKDTLMISRTPEVTCVVVDVKHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 26 - protein, 216 aa, Synthetic construct
APPVAGPSVFLFPPKPKDTLMISRTPEVTCVVVDVKHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 217 aa, Synthetic construct
APEFLGGPSVFLFPPKPKDTLMISRTPEVTCVVVDVSQEDPEVQFNWYVDGVEVHNAKTKPREEEFNSTYRVVSVLTVLHQDWLNGKEYKCKVSNKGLPSSIEKTISKAKGQPREPQVYTLPPSQEEMTKNQVSLTCDVSGFYPSDIAVEWESDGQPENNYKTTPPVLDSDGSFFLYSRLTVDKSRWEEGDVFSCSVMHEALHNHYTQKSLSLSLGK

SEQ ID NO: 28 - protein, 217 aa, Synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEQMTKNQVKLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 216 aa, Synthetic construct
APPVAGPSVFLFPPKPKDTLMISRTPEVTCVVVDVSHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 30 - protein, 216 aa, Synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 216 aa, Synthetic construct
APPVAGPSVFLFPPKPKDTLMISRTPEVTCVVVDVKHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 32 - protein, 216 aa, Synthetic construct
APPVAGPSVFLFPPKPKDTLMISRTPEVTCVVVDVKHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 216 aa, Synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 34 - protein, 216 aa, Synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 35 - protein, 216 aa, Synthetic construct
APPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 216 aa, Synthetic construct; identical to SEQ ID NO: 13

SEQ ID NO: 37 - protein, 216 aa, Synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 38 - protein, 216 aa, Synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 39 - protein, 216 aa, Synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 40 - protein, 216 aa, Synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 41 - protein, 216 aa, Synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 42 - protein, 216 aa, Synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 43 - protein, 217 aa, Synthetic construct
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 44 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 217 aa, Synthetic construct; identical to SEQ ID NO: 43

SEQ ID NO: 46 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 217 aa, Synthetic construct
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 48 - protein, 217 aa, Synthetic construct; identical to SEQ ID NO: 46

SEQ ID NO: 49 - protein, 217 aa, Synthetic construct
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 50 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 51 - protein, 217 aa, Synthetic construct
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 52 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 217 aa, Synthetic construct
APELLGGPSVFLFPPKPKDTLMISRTPEVTCVVVDVSHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 54 - protein, 217 aa, Synthetic construct
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 217 aa, Synthetic construct
APELLGGPSVFLFPPKPKDTLMISRTPEVTCVVVDVSHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 56 - protein, 217 aa, Synthetic construct
APELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 217 aa, Synthetic construct
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 58 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 59 - protein, 217 aa, Synthetic construct
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 60 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 61 - protein, 217 aa, Synthetic construct
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 62 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 63 - protein, 217 aa, Synthetic construct
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 64 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 65 - protein, 217 aa, Synthetic construct
APELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 66 - protein, 217 aa, Synthetic construct
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 217 aa, Synthetic construct
APELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 68 - protein, 217 aa, Synthetic construct
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 217 aa, Synthetic construct
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 70 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 217 aa, Synthetic construct; identical to SEQ ID NO: 43

SEQ ID NO: 72 - protein, 217 aa, Synthetic construct; identical to SEQ ID NO: 66

SEQ ID NO: 73 - protein, 217 aa, Synthetic construct; identical to SEQ ID NO: 43

SEQ ID NO: 74 - protein, 217 aa, Synthetic construct; identical to SEQ ID NO: 68

SEQ ID NO: 75 - protein, 217 aa, Synthetic construct; identical to SEQ ID NO: 43

SEQ ID NO: 76 - protein, 217 aa, Synthetic construct; identical to SEQ ID NO: 70

SEQ ID NO: 77 - protein, 217 aa, Synthetic construct; identical to SEQ ID NO: 65

SEQ ID NO: 78 - protein, 217 aa, Synthetic construct; identical to SEQ ID NO: 44

SEQ ID NO: 79 - protein, 217 aa, Synthetic construct; identical to SEQ ID NO: 67

SEQ ID NO: 80 - protein, 217 aa, Synthetic construct; identical to SEQ ID NO: 44

SEQ ID NO: 81 - protein, 217 aa, Synthetic construct; identical to SEQ ID NO: 69

SEQ ID NO: 82 - protein, 217 aa, Synthetic construct; identical to SEQ ID NO: 44

SEQ ID NO: 83 - protein, 217 aa, Synthetic construct; identical to SEQ ID NO: 65

SEQ ID NO: 84 - protein, 217 aa, Synthetic construct; identical to SEQ ID NO: 68

SEQ ID NO: 85 - protein, 217 aa, Synthetic construct; identical to SEQ ID NO: 65

SEQ ID NO: 86 - protein, 217 aa, Synthetic construct; identical to SEQ ID NO: 70

SEQ ID NO: 87 - protein, 217 aa, Synthetic construct; identical to SEQ ID NO: 67

SEQ ID NO: 88 - protein, 217 aa, Synthetic construct; identical to SEQ ID NO: 66

SEQ ID NO: 89 - protein, 217 aa, Synthetic construct; identical to SEQ ID NO: 69

SEQ ID NO: 90 - protein, 217 aa, Synthetic construct; identical to SEQ ID NO: 66

SEQ ID NO: 91 - protein, 217 aa, Synthetic construct; identical to SEQ ID NO: 67

SEQ ID NO: 92 - protein, 217 aa, Synthetic construct; identical to SEQ ID NO: 70

SEQ ID NO: 93 - protein, 217 aa, Synthetic construct; identical to SEQ ID NO: 69

SEQ ID NO: 94 - protein, 217 aa, Synthetic construct; identical to SEQ ID NO: 68

SEQ ID NO: 95 - protein, 217 aa, Synthetic construct
APELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 96 - protein, 217 aa, Synthetic construct
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 97 - protein, 217 aa, Synthetic construct
APELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 98 - protein, 217 aa, Synthetic construct
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 99 - protein, 217 aa, Synthetic construct
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 100 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 101 - protein, 217 aa, Synthetic construct; identical to SEQ ID NO: 57

SEQ ID NO: 102 - protein, 217 aa, Synthetic construct; identical to SEQ ID NO: 96

SEQ ID NO: 103 - protein, 217 aa, Synthetic construct; identical to SEQ ID NO: 57

SEQ ID NO: 104 - protein, 217 aa, Synthetic construct; identical to SEQ ID NO: 98

SEQ ID NO: 105 - protein, 217 aa, Synthetic construct; identical to SEQ ID NO: 57

SEQ ID NO: 106 - protein, 217 aa, Synthetic construct; identical to SEQ ID NO: 100

SEQ ID NO: 107 - protein, 217 aa, Synthetic construct; identical to SEQ ID NO: 95

SEQ ID NO: 108 - protein, 217 aa, Synthetic construct; identical to SEQ ID NO: 58

SEQ ID NO: 109 - protein, 217 aa, Synthetic construct; identical to SEQ ID NO: 97

SEQ ID NO: 110 - protein, 217 aa, Synthetic construct; identical to SEQ ID NO: 58

SEQ ID NO: 111 - protein, 217 aa, Synthetic construct; identical to SEQ ID NO: 99

SEQ ID NO: 112 - protein, 217 aa, Synthetic construct; identical to SEQ ID NO: 58

SEQ ID NO: 113 - protein, 217 aa, Synthetic construct; identical to SEQ ID NO: 95

SEQ ID NO: 114 - protein, 217 aa, Synthetic construct; identical to SEQ ID NO: 98

SEQ ID NO: 115 - protein, 217 aa, Synthetic construct; identical to SEQ ID NO: 95

SEQ ID NO: 116 - protein, 217 aa, Synthetic construct; identical to SEQ ID NO: 100

SEQ ID NO: 117 - protein, 217 aa, Synthetic construct; identical to SEQ ID NO: 97

SEQ ID NO: 118 - protein, 217 aa, Synthetic construct; identical to SEQ ID NO: 96

SEQ ID NO: 119 - protein, 217 aa, Synthetic construct; identical to SEQ ID NO: 99

SEQ ID NO: 120 - protein, 217 aa, Synthetic construct; identical to SEQ ID NO: 96

SEQ ID NO: 121 - protein, 217 aa, Synthetic construct; identical to SEQ ID NO: 97

SEQ ID NO: 122 - protein, 217 aa, Synthetic construct; identical to SEQ ID NO: 100

SEQ ID NO: 123 - protein, 217 aa, Synthetic construct; identical to SEQ ID NO: 99

SEQ ID NO: 124 - protein, 217 aa, Synthetic construct; identical to SEQ ID NO: 98

SEQ ID NO: 125 - protein, 217 aa, Synthetic construct
APELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 126 - protein, 217 aa, Synthetic construct
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 127 - protein, 217 aa, Synthetic construct
APELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 128 - protein, 217 aa, Synthetic construct
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 129 - protein, 217 aa, Synthetic construct
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 130 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 131 - protein, 217 aa, Synthetic construct; identical to SEQ ID NO: 61

SEQ ID NO: 132 - protein, 217 aa, Synthetic construct; identical to SEQ ID NO: 126

SEQ ID NO: 133 - protein, 217 aa, Synthetic construct; identical to SEQ ID NO: 61

SEQ ID NO: 134 - protein, 217 aa, Synthetic construct; identical to SEQ ID NO: 128

SEQ ID NO: 135 - protein, 217 aa, Synthetic construct; identical to SEQ ID NO: 61

SEQ ID NO: 136 - protein, 217 aa, Synthetic construct; identical to SEQ ID NO: 130

SEQ ID NO: 137 - protein, 217 aa, Synthetic construct; identical to SEQ ID NO: 125

SEQ ID NO: 138 - protein, 217 aa, Synthetic construct; identical to SEQ ID NO: 62

SEQ ID NO: 139 - protein, 217 aa, Synthetic construct; identical to SEQ ID NO: 127

SEQ ID NO: 140 - protein, 217 aa, Synthetic construct; identical to SEQ ID NO: 62

SEQ ID NO: 141 - protein, 217 aa, Synthetic construct; identical to SEQ ID NO: 129

SEQ ID NO: 142 - protein, 217 aa, Synthetic construct; identical to SEQ ID NO: 62

SEQ ID NO: 143 - protein, 217 aa, Synthetic construct; identical to SEQ ID NO: 125

SEQ ID NO: 144 - protein, 217 aa, Synthetic construct; identical to SEQ ID NO: 128

SEQ ID NO: 145 - protein, 217 aa, Synthetic construct; identical to SEQ ID NO: 125

SEQ ID NO: 146 - protein, 217 aa, Synthetic construct; identical to SEQ ID NO: 130

SEQ ID NO: 147 - protein, 217 aa, Synthetic construct; identical to SEQ ID NO: 127

SEQ ID NO: 148 - protein, 217 aa, Synthetic construct; identical to SEQ ID NO: 126

SEQ ID NO: 149 - protein, 217 aa, Synthetic construct; identical to SEQ ID NO: 129

SEQ ID NO: 150 - protein, 217 aa, Synthetic construct; identical to SEQ ID NO: 126

SEQ ID NO: 151 - protein, 217 aa, Synthetic construct; identical to SEQ ID NO: 127

SEQ ID NO: 152 - protein, 217 aa, Synthetic construct; identical to SEQ ID NO: 130

SEQ ID NO: 153 - protein, 217 aa, Synthetic construct; identical to SEQ ID NO: 129

SEQ ID NO: 154 - protein, 217 aa, Synthetic construct; identical to SEQ ID NO: 128

SEQ ID NO: 155 - protein, 98 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 156 - protein, 98 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKV

SEQ ID NO: 157 - protein, 98 aa, Synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 158 - protein, 98 aa, Synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTV

SEQ ID NO: 159 - protein, 98 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 160 - protein, 98 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSDTKVDKRV

SEQ ID NO: 161 - protein, 15 aa, Synthetic construct
EPKSCDKTHTCPPCP

SEQ ID NO: 162 - protein, 12 aa, Synthetic construct
ERKCCVECPPCP

SEQ ID NO: 163 - protein, 12 aa, Synthetic construct
ESKYGPPCPSCP

SEQ ID NO: 164 - protein, 107 aa, Synthetic construct [sequence omitted]

SEQ ID NO: 165 - protein, 106 aa, Synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 166 - protein, 5 aa, Synthetic construct
GGGGS

SEQ ID NO: 167 - protein, 10 aa, Synthetic construct
GGGGSGGGGS

SEQ ID NO: 168 - protein, 15 aa, Synthetic construct
GGGGSGGGGSGGGGS

SEQ ID NO: 169 - protein, 20 aa, Synthetic construct
GGGGSGGGGSGGGGSGGGGS

SEQ ID NO: 170 - protein, 25 aa, Synthetic construct
GGGGSGGGGSGGGGSGGGGSGGGGS

SEQ ID NO: 171 - protein, 30 aa, Synthetic construct
GGGGSGGGGSGGGGSGGGGSGGGGSGGGGS

SEQ ID NO: 172 - protein, 35 aa, Synthetic construct
GGGGSGGGGSGGGGSGGGGSGGGGSGGGGSGGGGS

SEQ ID NO: 173 - protein, 5 aa, Synthetic construct
GGGGA

SEQ ID NO: 174 - protein, 10 aa, Synthetic construct
GGGGAGGGGA

SEQ ID NO: 175 - protein, 15 aa, Synthetic construct
GGGGAGGGGAGGGGA

SEQ ID NO: 176 - protein, 20 aa, Synthetic construct
GGGGAGGGGAGGGGAGGGGA

SEQ ID NO: 177 - protein, 25 aa, Synthetic construct
GGGGAGGGGAGGGGAGGGGAGGGGA

SEQ ID NO: 178 - protein, 30 aa, Synthetic construct
GGGGAGGGGAGGGGAGGGGAGGGGAGGGGA

SEQ ID NO: 179 - protein, 35 aa, Synthetic construct
GGGGAGGGGAGGGGAGGGGAGGGGAGGGGAGGGGA

SEQ ID NO: 180 - protein, 30 aa, Synthetic construct
DPALVHQRPAPPGGGGSGGGGSGGGGSGGG

SEQ ID NO: 181 - protein, 5 aa, Synthetic construct
GKPGS

SEQ ID NO: 182 - protein, 25 aa, Synthetic construct
GKPGSGKPGSGKPGSGKPGSGKPGS

SEQ ID NO: 183 - protein, 30 aa, Synthetic construct
GKPGSGKPGSGKPGSGKPGSGKPGSGKPGS

SEQ ID NO: 184 - protein, 5 aa, Synthetic construct
GGGES

SEQ ID NO: 185 - protein, 9 aa, Synthetic construct
KTHTCPPCP

SEQ ID NO: 186 - protein, 15 aa, Synthetic construct
EPKSSDKTHTCPPCP

SEQ ID NO: 187 - protein, 19 aa, Synthetic construct
GGGGSGGGGSKTHTCPPCP

SEQ ID NO: 188 - protein, 19 aa, Synthetic construct
GKPGSGKPGSKTHTCPPCP

SEQ ID NO: 189 - protein, 14 aa, Synthetic construct
GKPGSKTHTCPPCP

SEQ ID NO: 190 - protein, 5 aa, Synthetic construct
EPKSC

SEQ ID NO: 191 - protein, 15 aa, Synthetic construct
EPKSCGGGGSGGGGS

SEQ ID NO: 192 - protein, 15 aa, Synthetic construct
EPKSCGKPGSGKPGS

SEQ ID NO: 193 - protein, 10 aa, Synthetic construct
EPKSCGKPGS

SEQ ID NO: 194 - protein, 20 aa, Synthetic construct
GKPGSGKPGSGKPGSGKPGS

(19) United States
(12) Patent Application Publication
(10) Pub. No.: US 2024/0360220 A1
(43) Pub. Date: Oct. 31, 2024

(54) ORTHOGONAL MULTIMERIC PROTEINS

(71) Applicant: Xencor, Inc., Pasadena, CA (US)

(72) Inventors: Gregory Moore, Azusa, CA (US); John R. Desjarlais, Pasadena, CA (US); Bhavika R. Patel, Artesia, CA (US)

(21) Appl. No.: 18/647,843

(22) Filed: Apr. 26, 2024

Related U.S. Application Data
(60) Provisional application No. 63/498,958, filed on Apr. 28, 2023.

(51) Int. Cl. C07K 16/28

(52) U.S. Cl. CPC C07K 16/283; C07K 2317/51; C07K 2317/522; C07K 2317/524; C07K 2317/526; C07K 2317/53; C07K 2317/55

(57) ABSTRACT

Provided herein are multimeric proteins that reduce the promiscuous pairing of monomers of the multimeric protein and/or promote the proper pairing of monomers of the multimeric proteins, as well as methods of using such multimeric proteins.

### CROSS-REFERENCE

This application claims priority to U.S. Provisional Application No. 63/498,958, filed on Apr. 28, 2023, which is incorporated herein by reference in its entirety.

### SEQUENCE LISTING

This application contains a Sequence Listing which has been submitted electronically in XML format and is hereby incorporated by reference in its entirety. The XML copy, created Apr. 26, 2024, is named “51096_4013_SL.xml” and is 203,720 bytes in size.

### BACKGROUND OF THE INVENTION

Production of bispecific antibodies in IgG format is challenging, as antibody heavy chains bind antibody light chains in a relatively promiscuous manner. As a result of this promiscuous pairing, concomitant expression of, e.g., two antibody heavy chains and two antibody light chains naturally leads to heavy chain homodimerization and/or scrambling of heavy chain/light chain pairings. While there are numerous approaches to circumvent the problem of heavy chain homodimerization, circumventing the scrambling of heavy chain/light chain pairing has been more difficult due to the complex multidomain heterodimeric interactions within antibody Fabs.

Similarly, the production of multimeric proteins that include two heavy chains and two light chains can result in heavy chain homodimerization and/or scrambling of heavy chain/light chain pairings. As such, there remains an unmet need for novel multimeric proteins with the ability to overcome the promiscuous pairing of two heavy chains and two light chains.

### BRIEF SUMMARY OF THE INVENTION

In one aspect, the present disclosure provides a multimeric protein, comprising: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain, wherein either the first monomer and the second monomer or the third monomer and the fourth monomer comprise a set of CH1:CL electrostatic variants, wherein the set of CH1:CL electrostatic variants comprises amino acid substitutions at amino acid residues K213/K218:D122/E123, wherein numbering is according to EU numbering.

In a further embodiment and in accordance with the above, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering.

In a further embodiment and in accordance with any of the above, the set of CH1:CL electrostatic variants comprises amino acid substitutions K213E/K218D:D122K/E123K, wherein numbering is according to EU numbering.

In a further embodiment and in accordance with any of the above, the first monomer and the second monomer do not comprise the set of CH1:CL electrostatic variants.

In a further embodiment and in accordance with any of the above, the third monomer and the fourth monomer do not comprise the set of CH1:CL electrostatic variants.

In a further embodiment and in accordance with any of the above, the first and/or second variant IgG Fc domains comprise one or more FcγRIIIA (CD16a) binding variant substitutions.

In a further embodiment and in accordance with the above, the one or more FcγRIIIA (CD16a) binding variant substitutions are selected from a group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with any of the above, the first and second variant IgG Fc domains comprise a set of FcγRIIIA (CD16a) binding variant substitutions selected from a group including: (i) S239D/I332E:S239D/I332E, (ii) S239D:S239D, (iii) 1332E: 1332E, (iv) WT:S239D/I332E, (v) WT:S239D, (vi) WT:1332E, (vii) S239D/I332E: WT, (viii) S239D:WT, (ix) 1332E:WT, (x) S239D/I332E:S239D, (xi) S239D/I332E:1332E, (xii) S239D:S239D/I332E, (xiii) 1332E:S239D/I332E, (xiv) S239D:1332E, and (xv) 1332E: S239D, wherein numbering is according to EU numbering.

In a further embodiment and in accordance with any of the above, the first and/or second variant IgG Fc domains comprise the FcγRIIIA (CD16a) binding variant substitutions of S239D/I332E, wherein numbering is according to EU numbering.

In a further embodiment and in accordance with any of the above, the first and second variant IgG Fc domains comprise a set of heterodimerization variants selected from a group including those depicted in FIGS. 13A-13F, wherein numbering is according to EU numbering.

In a further embodiment and in accordance with the above, the set of heterodimerization variants is selected from a group including: (i) S364K/E357Q:L368D/K370S, (ii) S364K: L368D/K370S, (iii) S364K:L368E/K370S, (iv) D401K:T411E/K360E/Q362E, and (v) T366W:T366S/L368A/Y407V, wherein numbering is according to EU numbering.

In a further embodiment and in accordance with any of the above, the first and second variant IgG Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant IgG Fc domains comprise one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first variant IgG Fc domain comprises amino acid variants S364K/E357Q/E233P/L234V/L235A/G236del/S267K, wherein the second variant IgG Fc domain comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second variant IgG Fc domains each further comprise amino acid variants M428L/N434S or M428L/N434A, wherein numbering is according to EU numbering.

In a further embodiment and in accordance with any of the above, the first variant IgG Fc domain is selected from a group including: (i) a first variant IgG1 Fc domain, (ii) a first variant IgG2 Fc domain, and (iii) a first variant IgG4 Fc domain.

In a further embodiment and in accordance with any of the above, the second variant IgG Fc domain is selected from a group including: (i) a second variant IgG1 Fc domain, (ii) a second variant IgG2 Fc domain, and (iii) a second variant IgG4 Fc domain.

In another aspect, a nucleic acid composition comprising nucleic acids encoding the first monomer, the second monomer, the third monomer, and the fourth monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain, wherein either the first monomer and the second monomer or the third monomer and the fourth monomer comprise a set of CH1:CL steric variants, wherein the set of CH1:CL steric variants comprises amino acid substitutions at amino acid residues selected from a group including: (i) A141:F118, (ii) A141:F116, and (iii) K147:S131, wherein numbering is according to EU numbering.

In a further embodiment and in accordance with the above, the set of CH1:CL steric variants comprises amino acid substitutions selected from a group including: (i) A141F:F118A, (ii) A141F:F116A, and (iii) K147S:S131K, wherein numbering is according to EU numbering.

In a further embodiment and in accordance with any of the above, the first monomer and the second monomer do not comprise the set of CH1:CL steric variants.

In a further embodiment and in accordance with any of the above, the third monomer and the fourth monomer do not comprise the set of CH1:CL steric variants.

In a further embodiment and in accordance with any of the above, the first and/or second variant IgG Fc domains comprise one or more FcγRIIIA (CD16a) binding variant substitutions.

In a further embodiment and in accordance with the above, the one or more FcγRIIIA (CD16a) binding variant substitutions are selected from a group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with any of the above, the first and second variant IgG Fc domains comprise a set of FcγRIIIA (CD16a) binding variant substitutions selected from a group including: (i) S239D/I332E:S239D/I332E, (ii) S239D:S239D, (iii) I332E:I332E, (iv) WT:S239D/I332E, (v) WT:S239D, (vi) WT:I332E, (vii) S239D/I332E: WT, (viii) S239D:WT, (ix) I332E:WT, (x) S239D/I332E:S239D, (xi) S239D/I332E:I332E, (xii) S239D:S239D/I332E, (xiii) I332E:S239D/I332E, (xiv) S239D:I332E, and (xv) I332E: S239D, wherein numbering is according to EU numbering.

In a further embodiment and in accordance with any of the above, the first and/or second variant IgG Fc domains comprise the FcγRIIIA (CD16a) binding variant substitutions of S239D/I332E, wherein numbering is according to EU numbering.

In a further embodiment and in accordance with any of the above, the first and second variant IgG Fc domains comprise a set of heterodimerization variants selected from a group including those depicted in FIGS. 13A-13F, wherein numbering is according to EU numbering.

In a further embodiment and in accordance with the above, the set of heterodimerization variants is selected from a group including: (i) S364K/E357Q:L368D/K370S, (ii) S364K: L368D/K370S, (iii) S364K:L368E/K370S, (iv) D401K:T411E/K360E/Q362E, and (v) T366W:T366S/L368A/Y407V, wherein numbering is according to EU numbering.

In a further embodiment and in accordance with any of the above, the first and second variant IgG Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant IgG Fc domains comprise one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first variant IgG Fc domain comprises amino acid variants S364K/E357Q/E233P/L234V/L235A/G236del/S267K, wherein the second variant IgG Fc domain comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second variant IgG Fc domains each further comprise amino acid variants M428L/N434S or M428L/N434A, wherein numbering is according to EU numbering.

In a further embodiment and in accordance with any of the above, the first variant IgG Fc domain is selected from a group including: (i) a first variant IgG1 Fc domain, (ii) a first variant IgG2 Fc domain, and (iii) a first variant IgG4 Fc domain.

In a further embodiment and in accordance with any of the above, the second variant IgG Fc domain is selected from a group including: (i) a second variant IgG1 Fc domain, (ii) a second variant IgG2 Fc domain, and (iii) a second variant IgG4 Fc domain.

In another aspect, a nucleic acid composition comprising nucleic acids encoding the first monomer, the second monomer, the third monomer, and the fourth monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain, wherein (i) the first monomer and the second monomer comprise a first set of VH:VL electrostatic variants comprising amino acid substitutions at amino acid residues Q39:Q38, and (ii) the third monomer and the fourth monomer comprise a second set of VH:VL electrostatic variants, comprising amino acid substitutions at amino acid residues Q39:Q38, wherein numbering is according to Kabat numbering.

In a further embodiment and in accordance with the above, the first set of VH:VL electrostatic variants and the second set of VH:VL electrostatic variants comprise amino acid substitutions selected from a group including: (i) Q39E:Q38K and Q39K:Q38E, respectively, (ii) Q39E:Q38K and Q39K:Q38D, respectively, (iii) Q39E:Q38K and Q39R:Q38E, respectively, (iv) Q39E:Q38K and Q39R:Q38D, respectively, (v) Q39E:Q38K and Q39E:Q38K, respectively, (vi) Q39E:Q38K and Q39E:Q38R, respectively, (vii) Q39E:Q38K and Q39D:Q38K, respectively, (viii) Q39E:Q38K and Q39D:Q38R, respectively, (ix) Q39E:Q38R and Q39K:Q38E, respectively, (x) Q39E:Q38R and Q39K:Q38D, respectively, (xi) Q39E:Q38R and Q39R:Q38E, respectively, (xii) Q39E:Q38R and Q39R:Q38D, respectively, (xiii) Q39E:Q38R and Q39E:Q38K, respectively, (xiv) Q39E:Q38R and Q39E:Q38R, respectively, (xv) Q39E:Q38R and Q39D:Q38K, respectively, (xvi) Q39E:Q38R and Q39D:Q38R, respectively, (xvii) Q39D:Q38K and Q39K:Q38E, respectively, (xviii) Q39D:Q38K and Q39K:Q38D, respectively, (xix) Q39D:Q38K and Q39R:Q38E, respectively, (xx) Q39D:Q38K and Q39R:Q38D, respectively, (xxi) Q39D:Q38K and Q39E:Q38K, respectively, (xxii) Q39D:Q38K and Q39E:Q38R, respectively, (xxiii) Q39D:Q38K and Q39D:Q38K, respectively, (xxiv) Q39D:Q38K and Q39D:Q38R, respectively, (xxv) Q39D:Q38R and Q39K:Q38E, respectively, (xxvi) Q39D:Q38R and Q39K:Q38D, respectively, (xxvii) Q39D:Q38R and Q39R:Q38E, respectively, (xxviii) Q39D:Q38R and Q39R:Q38D, respectively, (xxix) Q39D:Q38R and Q39E:Q38K, respectively, (xxx) Q39D:Q38R and Q39E:Q38R, respectively, (xxxi) Q39D:Q38R and Q39D:Q38K, respectively, (xxxii) Q39D:Q38R and Q39D:Q38R, respectively, (xxxiii) Q39K:Q38E and Q39K:Q38E, respectively, (xxxiv) Q39K:Q38E and Q39K:Q38D, respectively, (xxxv) Q39K:Q38E and Q39R:Q38E, respectively, (xxxvi) Q39K:Q38E and Q39R:Q38D, respectively, (xxxvii) Q39K:Q38E and Q39E:Q38K, respectively, (xxxviii) Q39K:Q38E and Q39E:Q38R, respectively, (xxxix) Q39K:Q38E and Q39D:Q38K, respectively, (xl) Q39K:Q38E and Q39D:Q38R, respectively, (xli) Q39R:Q38E and Q39K:Q38E, respectively, (xlii) Q39R:Q38E and Q39K:Q38D, respectively, (xliii) Q39R:Q38E and Q39R:Q38E, respectively, (xliv) Q39R:Q38E and Q39R:Q38D, respectively, (xlv) Q39R:Q38E and Q39E:Q38K, respectively, (xlvi) Q39R:Q38E and Q39E:Q38R, respectively, (xlvii) Q39R:Q38E and Q39D:Q38K, respectively, (xlviii) Q39R:Q38E and Q39D:Q38R, respectively, (xlix) Q39K:Q38D and Q39K:Q38E, respectively, (1) Q39K:Q38D and Q39K:Q38D, respectively, (li) Q39K:Q38D and Q39R:Q38E, respectively, (lii) Q39K:Q38D and Q39R:Q38D, respectively, (liii) Q39K:Q38D and Q39E:Q38K, respectively, (liv) Q39K:Q38D and Q39E:Q38R, respectively, (lv) Q39K:Q38D and Q39D:Q38K, respectively, (lvi) Q39K:Q38D and Q39D:Q38R, respectively, (lvii) Q39R:Q38D and Q39K:Q38E, respectively, (lviii) Q39R:Q38D and Q39K:Q38D, respectively, (lix) Q39R:Q38D and Q39R:Q38E, respectively, (lx) Q39R:Q38D and Q39R:Q38D, respectively, (lxi) Q39R:Q38D and Q39E:Q38K, respectively, (lxii) Q39R:Q38D and Q39E:Q38R, respectively, (lxiii) Q39R:Q38D and Q39D:Q38K, respectively, and (lxiv) Q39R:Q38D and Q39D:Q38R, respectively, wherein numbering is according to Kabat numbering.

In a further embodiment and in accordance with any of the above, the first set of VH:VL electrostatic variants comprises amino acid substitutions Q39E:Q38K, and the second set of VH:VL electrostatic variants comprises amino acid substitutions Q39K:Q38E, wherein numbering is according to Kabat numbering.

In a further embodiment and in accordance with any of the above, the first set of VH:VL electrostatic variants comprises amino acid substitutions Q39K:Q38E, and the second set of VH:VL electrostatic variants comprises amino acid substitutions Q39E:Q38K, wherein numbering is according to Kabat numbering.

In a further embodiment and in accordance with any of the above, the first and/or second variant IgG Fc domains comprise one or more FcγRIIIA (CD16a) binding variant substitutions.

In a further embodiment and in accordance with the above, the one or more FcγRIIIA (CD16a) binding variant substitutions are selected from a group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with any of the above, the first and second variant IgG Fc domains comprise a set of FcγRIIIA (CD16a) binding variant substitutions selected from a group including: (i) S239D/I332E:S239D/I332E, (ii) S239D:S239D, (iii) I332E:I332E, (iv) WT:S239D/I332E, (v) WT:S239D, (vi) WT:I332E, (vii) S239D/I332E: WT, (viii) S239D:WT, (ix) I332E:WT, (x) S239D/I332E:S239D, (xi) S239D/I332E:I332E, (xii) S239D:S239D/I332E, (xiii) I332E:S239D/I332E, (xiv) S239D:I332E, and (xv) I332E: S239D, wherein numbering is according to EU numbering.

In a further embodiment and in accordance with any of the above, the first and/or second variant IgG Fc domains comprise the FcγRIIIA (CD16a) binding variant substitutions of S239D/I332E, wherein numbering is according to EU numbering.

In a further embodiment and in accordance with any of the above, the first and second variant IgG Fc domains comprise a set of heterodimerization variants selected from a group including those depicted in FIGS. 13A-13F, wherein numbering is according to EU numbering.

In a further embodiment and in accordance with the above, the set of heterodimerization variants is selected from a group including: (i) S364K/E357Q:L368D/K370S, (ii) S364K: L368D/K370S, (iii) S364K:L368E/K370S, (iv) D401K:T411E/K360E/Q362E, and (v) T366W: T366S/L368A/Y407V, wherein numbering is according to EU numbering.

In a further embodiment and in accordance with any of the above, the first and second variant IgG Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant IgG Fc domains comprise one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first variant IgG Fc domain comprises amino acid variants S364K/E357Q/E233P/L234V/L235A/G236del/S267K, wherein the second variant IgG Fc domain comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second variant IgG Fc domains each further comprise amino acid variants M428L/N434S or M428L/N434A, wherein numbering is according to EU numbering.

In a further embodiment and in accordance with any of the above, the first variant IgG Fc domain is selected from a group including: (i) a first variant IgG1 Fc domain, (ii) a first variant IgG2 Fc domain, and (iii) a first variant IgG4 Fc domain.

In a further embodiment and in accordance with any of the above, the second variant IgG Fc domain is selected from a group including: (i) a second variant IgG1 Fc domain, (ii) a second variant IgG2 Fc domain, and (iii) a second variant IgG4 Fc domain.

In another aspect, a nucleic acid composition comprising nucleic acids encoding the first monomer, the second monomer, the third monomer, and the fourth monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain, wherein (i) the first monomer and the second monomer comprise a first set of VH:VL electrostatic variants and a set of CH1:CL electrostatic variants, and (ii) the third monomer and the fourth monomer comprise a second set of VH:VL electrostatic variants and a set of CH1:CL steric variants.

In a further embodiment and in accordance with the above, wherein: (i) the set of CH1:CL electrostatic variants comprises amino acid substitutions at amino acid residues K213/K218:D122/E123, wherein numbering is according to EU numbering; (ii) the set of CH1:CL steric variants comprises amino acid substitutions at amino acid residues selected from a group including: (a) A141:F116, (b) A141:F118, and (c) K147:S131, wherein numbering is according to EU numbering; and (iii) the first set of VH:VL electrostatic variants and the second set of VH:VL electrostatic variants each comprise amino acid substitutions at amino acid residues Q39:Q38, wherein numbering is according to Kabat numbering.

In a further embodiment and in accordance with any of the above, wherein: (a) the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering; (b) the set of CH1:CL steric variants comprises amino acid substitutions selected from a group including: (i) A141F:F118A, (ii) A141F:F116A, and (iii) K147S:S131K, wherein numbering is according to EU numbering; and (c) the first set of VH:VL electrostatic variants and the second set of VH:VL electrostatic variants comprise amino acid substitutions selected from a group including: (i) Q39E:Q38K and Q39K:Q38E, respectively, (ii) Q39E:Q38K and Q39K:Q38D, respectively, (iii) Q39E:Q38K and Q39R:Q38E, respectively, (iv) Q39E:Q38K and Q39R:Q38D, respectively, (v) Q39E:Q38K and Q39E:Q38K, respectively, (vi) Q39E:Q38K and Q39E:Q38R, respectively, (vii) Q39E:Q38K and Q39D:Q38K, respectively, (viii) Q39E:Q38K and Q39D:Q38R, respectively, (ix) Q39E:Q38R and Q39K:Q38E, respectively, (x) Q39E:Q38R and Q39K:Q38D, respectively, (xi) Q39E:Q38R and Q39R:Q38E, respectively, (xii) Q39E:Q38R and Q39R:Q38D, respectively, (xiii) Q39E:Q38R and Q39E:Q38K, respectively, (xiv) Q39E:Q38R and Q39E:Q38R, respectively, (xv) Q39E:Q38R and Q39D:Q38K, respectively, (xvi) Q39E:Q38R and Q39D:Q38R, respectively, (xvii) Q39D:Q38K and Q39K:Q38E, respectively, (xviii) Q39D:Q38K and Q39K:Q38D, respectively, (xix) Q39D:Q38K and Q39R:Q38E, respectively, (xx) Q39D:Q38K and Q39R:Q38D, respectively, (xxi) Q39D:Q38K and Q39E:Q38K, respectively, (xxii) Q39D:Q38K and Q39E:Q38R, respectively, (xxiii) Q39D:Q38K and Q39D:Q38K, respectively, (xxiv) Q39D:Q38K and Q39D:Q38R, respectively, (xxv) Q39D:Q38R and Q39K:Q38E, respectively, (xxvi) Q39D:Q38R and Q39K:Q38D, respectively, (xxvii) Q39D:Q38R and Q39R:Q38E, respectively, (xxviii) Q39D:Q38R and Q39R:Q38D, respectively, (xxix) Q39D:Q38R and Q39E:Q38K, respectively, (xxx) Q39D:Q38R and Q39E:Q38R, respectively, (xxxi) Q39D:Q38R and Q39D:Q38K, respectively, (xxxii) Q39D:Q38R and Q39D:Q38R, respectively, (xxxiii) Q39K:Q38E and Q39K:Q38E, respectively, (xxxiv) Q39K:Q38E and Q39K:Q38D, respectively, (xxxv) Q39K:Q38E and Q39R:Q38E, respectively, (xxxvi) Q39K:Q38E and Q39R:Q38D, respectively, (xxxvii) Q39K:Q38E and Q39E:Q38K, respectively, (xxxviii) Q39K:Q38E and Q39E:Q38R, respectively, (xxxix) Q39K:Q38E and Q39D:Q38K, respectively, (xl) Q39K:Q38E and Q39D:Q38R, respectively, (xli) Q39R:Q38E and Q39K:Q38E, respectively, (xlii) Q39R:Q38E and Q39K:Q38D, respectively, (xliii) Q39R:Q38E and Q39R:Q38E, respectively, (xliv) Q39R:Q38E and Q39R:Q38D, respectively, (xlv) Q39R:Q38E and Q39E:Q38K, respectively, (xlvi) Q39R:Q38E and Q39E:Q38R, respectively, (xlvii) Q39R:Q38E and Q39D:Q38K, respectively, (xlviii) Q39R:Q38E and Q39D:Q38R, respectively, (xlix) Q39K:Q38D and Q39K:Q38E, respectively, (1) Q39K:Q38D and Q39K:Q38D, respectively, (li) Q39K:Q38D and Q39R:Q38E, respectively, (lii) Q39K:Q38D and Q39R:Q38D, respectively, (liii) Q39K:Q38D and Q39E:Q38K, respectively, (liv) Q39K:Q38D and Q39E:Q38R, respectively, (lv) Q39K:Q38D and Q39D:Q38K, respectively, (lvi) Q39K:Q38D and Q39D:Q38R, respectively, (lvii) Q39R:Q38D and Q39K:Q38E, respectively, (lviii) Q39R:Q38D and Q39K:Q38D, respectively, (lix) Q39R:Q38D and Q39R:Q38E, respectively, (lx) Q39R:Q38D and Q39R:Q38D, respectively, (lxi) Q39R:Q38D and Q39E:Q38K, respectively, (lxii) Q39R:Q38D and Q39E:Q38R, respectively, (lxiii) Q39R:Q38D and Q39D:Q38K, respectively, and (lxiv) Q39R:Q38D and Q39D:Q38R, respectively, wherein numbering is according to Kabat numbering.

In a further embodiment and in accordance with any of the above, the set of CH1:CL electrostatic variants comprises amino acid substitutions K213E/K218D:D122K/E123K, wherein numbering is according to EU numbering.

In a further embodiment and in accordance with any of the above, the first set of VH:VL electrostatic variants comprises amino acid substitutions Q39E:Q38K, and the second set of VH:VL electrostatic variants comprises amino acid substitutions Q39K:Q38E, wherein numbering is according to Kabat numbering.

In a further embodiment and in accordance with any of the above, the first set of VH:VL electrostatic variants comprises amino acid substitutions Q39K:Q38E, and the second set of VH:VL electrostatic variants comprises amino acid substitutions Q39E:Q38K, wherein numbering is according to Kabat numbering.

In a further embodiment and in accordance with any of the above, the first and/or second variant IgG Fc domains comprise one or more FcγRIIIA (CD16a) binding variant substitutions.

In a further embodiment and in accordance with the above, the one or more FcγRIIIA (CD16a) binding variant substitutions are selected from a group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with any of the above, the first and second variant IgG Fc domains comprise a set of FcγRIIIA (CD16a) binding variant substitutions selected from a group including: (i) S239D/I332E:S239D/I332E, (ii) S239D:S239D, (iii) 1332E: 1332E, (iv) WT:S239D/I332E, (v) WT:S239D, (vi) WT:1332E, (vii) S239D/I332E: WT, (viii) S239D:WT, (ix) 1332E:WT, (x) S239D/I332E:S239D, (xi) S239D/I332E:1332E, (xii) S239D:S239D/I332E, (xiii) 1332E:S239D/I332E, (xiv) S239D:1332E, and (xv) 1332E: S239D, wherein numbering is according to EU numbering.

In a further embodiment and in accordance with any of the above, the first and/or second variant IgG Fc domains comprise the FcγRIIIA (CD16a) binding variant substitutions of S239D/I332E, wherein numbering is according to EU numbering.

In a further embodiment and in accordance with any of the above, the first and second variant IgG Fc domains comprise a set of heterodimerization variants selected from a group including those depicted in FIGS. 13A-13F, wherein numbering is according to EU numbering.

In a further embodiment and in accordance with the above, the set of heterodimerization variants is selected from a group including: (i) S364K/E357Q:L368D/K370S, (ii) S364K: L368D/K370S, (iii) S364K:L368E/K370S, (iv) D401K:T411E/K360E/Q362E, and (v) T366W: T366S/L368A/Y407V, wherein numbering is according to EU numbering.

In a further embodiment and in accordance with any of the above, the first and second variant IgG Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant IgG Fc domains comprise one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first variant IgG Fc domain comprises amino acid variants S364K/E357Q/E233P/L234V/L235A/G236de1/S267K, wherein the second variant IgG Fc domain comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second variant IgG Fc domains each further comprise amino acid variants M428L/N434S or M428L/N434A, wherein numbering is according to EU numbering.

In a further embodiment and in accordance with any of the above, the first variant IgG Fc domain is selected from a group including: (i) a first variant IgG1 Fc domain, (ii) a first variant IgG2 Fc domain, and (iii) a first variant IgG4 Fc domain.

In a further embodiment and in accordance with any of the above, the second variant IgG Fc domain is selected from a group including: (i) a second variant IgG1 Fc domain, (ii) a second variant IgG2 Fc domain, and (iii) a second variant IgG4 Fc domain.

In another aspect, a nucleic acid composition comprising nucleic acids encoding the first monomer, the second monomer, the third monomer, and the fourth monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain, wherein (i) the VH:VL interface of the first monomer and the second monomer comprises a first set of VH:VL electrostatic variants comprising amino acid substitutions at amino acid residues Q39:Q38, and the VH:VL interface of the third monomer and the fourth monomer comprises a second set of VH:VL electrostatic variants comprising amino acid substitutions at amino acid residues Q39:Q38, wherein numbering is according to Kabat numbering, (ii) the CH1:CL interface of the first monomer and the second monomer and/or the CH1:CL interface of the third monomer and the fourth monomer comprises one or more amino acid substitutions, wherein the one or more amino acid substitutions comprises (1) a set of CH1:CL electrostatic variants, or (2) a set of CH1:CL steric variants, wherein numbering is according to EU numbering, and (iii) the multimeric protein has antigen binding affinity substantially equivalent to a corresponding multimeric protein lacking the amino acid substitutions at the VH:VL interfaces and the CH1:CL interface(s).

In a further embodiment and in accordance with the above, the CH1:CL interface of the first monomer and the second monomer does not comprise the one or more amino acid substitutions.

In a further embodiment and in accordance with the above, the CH1:CL interface of the third monomer and the fourth monomer does not comprise the one or more amino acid substitutions.

In a further embodiment and in accordance with any of the above, the CH1:CL interface comprises a set of CH1:CL electrostatic variants.

In a further embodiment and in accordance with the above, the set of CH1:CL electrostatic variants comprises amino acid substitutions at amino acid residues K213/K218:D122/E123, wherein numbering is according to EU numbering.

In a further embodiment and in accordance with any of the above, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering.

In a further embodiment and in accordance with any of the above, the set of CH1:CL electrostatic variants comprises amino acid substitutions K213E/K218D:D122K/E123K, wherein numbering is according to EU numbering.

In a further embodiment and in accordance with any of the above, the CH1:CL interface comprises a set of CH1:CL steric variants.

In a further embodiment and in accordance with the above, the set of CH1:CL steric variants comprises amino acid substitutions at amino acid residues selected from a group including: (i) A141:F118, (ii) A141:F116, and (iii) K147:S131, wherein numbering is according to EU numbering.

In a further embodiment and in accordance with any of the above, the set of CH1:CL steric variants comprises amino acid substitutions selected from a group including: (i) A141F:F118A, (ii) A141F:F116A, and (iii) K147S:S131K, wherein numbering is according to EU numbering.

In a further embodiment and in accordance with any of the above, wherein: (i) the CH1:CL interface of the first monomer and the second monomer comprises a set of CH1:CL electrostatic variants; and (ii) the CH1:CL interface of the third monomer and the fourth monomer comprises a set of CH1:CL steric variants.

In a further embodiment and in accordance with any of the above, wherein: (i) the CH1:CL interface of the first monomer and the second monomer comprises a set of CH1:CL steric variants; and (ii) the CH1:CL interface of the third monomer and the fourth monomer comprises a set of CH1:CL electrostatic variants.

In a further embodiment and in accordance with any of the above, wherein: (i) the set of CH1:CL electrostatic variants comprises amino acid substitutions at amino acid residues K213/K218:D122/E123, wherein numbering is according to EU numbering; and (ii) the set of CH1:CL steric variants comprises amino acid substitutions at amino acid residues selected from a group including: (i) A141:F118, (ii) A141:F116, and (iii) K147:S131, wherein numbering is according to EU numbering.

In a further embodiment and in accordance with any of the above, wherein: (i) the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering; and (ii) the set of CH1:CL steric variants comprises amino acid substitutions selected from a group including: (i) A141F:F118A, (ii) A141F:F116A, and (iii) K147S:S131K, wherein numbering is according to EU numbering.

In a further embodiment and in accordance with any of the above, the set of CH1:CL electrostatic variants comprises amino acid substitutions K213E/K218D:D122K/E123K, wherein numbering is according to EU numbering.

In a further embodiment and in accordance with any of the above, the first set of VH:VL electrostatic variants and the second set of VH:VL electrostatic variants comprise amino acid substitutions selected from a group including: (i) Q39E:Q38K and Q39K:Q38E, respectively, (ii) Q39E:Q38K and Q39K:Q38D, respectively, (iii) Q39E:Q38K and Q39R:Q38E, respectively, (iv) Q39E:Q38K and Q39R:Q38D, respectively, (v) Q39E:Q38K and Q39E:Q38K, respectively, (vi) Q39E:Q38K and Q39E:Q38R, respectively, (vii) Q39E:Q38K and Q39D:Q38K, respectively, (viii) Q39E:Q38K and Q39D:Q38R, respectively, (ix) Q39E:Q38R and Q39K:Q38E, respectively, (x) Q39E:Q38R and Q39K:Q38D, respectively, (xi) Q39E:Q38R and Q39R:Q38E, respectively, (xii) Q39E:Q38R and Q39R:Q38D, respectively, (xiii) Q39E:Q38R and Q39E:Q38K, respectively, (xiv) Q39E:Q38R and Q39E:Q38R, respectively, (xv) Q39E:Q38R and Q39D:Q38K, respectively, (xvi) Q39E:Q38R and Q39D:Q38R, respectively, (xvii) Q39D:Q38K and Q39K:Q38E, respectively, (xviii) Q39D:Q38K and Q39K:Q38D, respectively, (xix) Q39D:Q38K and Q39R:Q38E, respectively, (xx) Q39D:Q38K and Q39R:Q38D, respectively, (xxi) Q39D:Q38K and Q39E:Q38K, respectively, (xxii) Q39D:Q38K and Q39E:Q38R, respectively, (xxiii) Q39D:Q38K and Q39D:Q38K, respectively, (xxiv) Q39D:Q38K and Q39D:Q38R, respectively, (xxv) Q39D:Q38R and Q39K:Q38E, respectively, (xxvi) Q39D:Q38R and Q39K:Q38D, respectively, (xxvii) Q39D:Q38R and Q39R:Q38E, respectively, (xxviii) Q39D:Q38R and Q39R:Q38D, respectively, (xxix) Q39D:Q38R and Q39E:Q38K, respectively, (xxx) Q39D:Q38R and Q39E:Q38R, respectively, (xxxi) Q39D:Q38R and Q39D:Q38K, respectively, (xxxii) Q39D:Q38R and Q39D:Q38R, respectively, (xxxiii) Q39K:Q38E and Q39K:Q38E, respectively, (xxxiv) Q39K:Q38E and Q39K:Q38D, respectively, (xxxv) Q39K:Q38E and Q39R:Q38E, respectively, (xxxvi) Q39K:Q38E and Q39R:Q38D, respectively, (xxxvii) Q39K:Q38E and Q39E:Q38K, respectively, (xxxviii) Q39K:Q38E and Q39E:Q38R, respectively, (xxxix) Q39K:Q38E and Q39D:Q38K, respectively, (xl) Q39K:Q38E and Q39D:Q38R, respectively, (xli) Q39R:Q38E and Q39K:Q38E, respectively, (xlii) Q39R:Q38E and Q39K:Q38D, respectively, (xliii) Q39R:Q38E and Q39R:Q38E, respectively, (xliv) Q39R:Q38E and Q39R:Q38D, respectively, (xlv) Q39R:Q38E and Q39E:Q38K, respectively, (xlvi) Q39R:Q38E and Q39E:Q38R, respectively, (xlvii) Q39R:Q38E and Q39D:Q38K, respectively, (xlviii) Q39R:Q38E and Q39D:Q38R, respectively, (xlix) Q39K:Q38D and Q39K:Q38E, respectively, (1) Q39K:Q38D and Q39K:Q38D, respectively, (li) Q39K:Q38D and Q39R:Q38E, respectively, (lii) Q39K:Q38D and Q39R:Q38D, respectively, (liii) Q39K:Q38D and Q39E:Q38K, respectively, (liv) Q39K:Q38D and Q39E:Q38R, respectively, (lv) Q39K:Q38D and Q39D:Q38K, respectively, (lvi) Q39K:Q38D and Q39D:Q38R, respectively, (lvii) Q39R:Q38D and Q39K:Q38E, respectively, (lviii) Q39R:Q38D and Q39K:Q38D, respectively, (lix) Q39R:Q38D and Q39R:Q38E, respectively, (lx) Q39R:Q38D and Q39R:Q38D, respectively, (lxi) Q39R:Q38D and Q39E:Q38K, respectively, (lxii) Q39R:Q38D and Q39E:Q38R, respectively, (lxiii) Q39R:Q38D and Q39D:Q38K, respectively, and (lxiv) Q39R:Q38D and Q39D:Q38R, respectively, wherein numbering is according to Kabat numbering.

In a further embodiment and in accordance with the above, the first set of VH:VL electrostatic variants comprises amino acid substitutions Q39E:Q38K, and the second set of VH:VL electrostatic variants comprises amino acid substitutions Q39K:Q38E, wherein numbering is according to Kabat numbering.

In a further embodiment and in accordance with the above, the first set of VH:VL electrostatic variants comprises amino acid substitutions Q39K:Q38E, and the second set of VH:VL electrostatic variants comprises amino acid substitutions Q39E:Q38K, wherein numbering is according to Kabat numbering.

In a further embodiment and in accordance with any of the above, the first and/or second variant IgG Fc domains comprise one or more FcγRIIIA (CD16a) binding variant substitutions.

In a further embodiment and in accordance with the above, the one or more FcγRIIIA (CD16a) binding variant substitutions are selected from a group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with any of the above, the first and second variant IgG Fc domains comprise a set of FcγRIIIA (CD16a) binding variant substitutions selected from a group including: (i) S239D/I332E:S239D/I332E, (ii) S239D:S239D, (iii) I332E:I332E, (iv) WT:S239D/I332E, (v) WT:S239D, (vi) WT:I332E, (vii) S239D/I332E: WT, (viii) S239D:WT, (ix) I332E:WT, (x) S239D/I332E:S239D, (xi) S239D/I332E:I332E, (xii) S239D:S239D/I332E, (xiii) I332E:S239D/I332E, (xiv) S239D:I332E, and (xv) I332E: S239D, wherein numbering is according to EU numbering.

In a further embodiment and in accordance with any of the above, the first and/or second variant IgG Fc domains comprise the FcγRIIIA (CD16a) binding variant substitutions of S239D/I332E, wherein numbering is according to EU numbering.

In a further embodiment and in accordance with any of the above, the first and second variant IgG Fc domains comprise a set of heterodimerization variants selected from a group including those depicted in FIGS. 13A-13F, wherein numbering is according to EU numbering.

In a further embodiment and in accordance with the above, the set of heterodimerization variants is selected from a group including: (i) S364K/E357Q:L368D/K370S, (ii) S364K: L368D/K370S, (iii) S364K:L368E/K370S, (iv) D401K:T411E/K360E/Q362E, and (v) T366W: T366S/L368A/Y407V, wherein numbering is according to EU numbering.

In a further embodiment and in accordance with any of the above, the first and second variant IgG Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant IgG Fc domains comprise one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first variant IgG Fc domain comprises amino acid variants S364K/E357Q/E233P/L234V/L235A/G236del/S267K, wherein the second variant IgG Fc domain comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second variant IgG Fc domains each further comprise amino acid variants M428L/N434S or M428L/N434A, wherein numbering is according to EU numbering.

In a further embodiment and in accordance with any of the above, the first variant IgG Fc domain is selected from a group including: (i) a first variant IgG1 Fc domain, (ii) a first variant IgG2 Fc domain, and (iii) a first variant IgG4 Fc domain.

In a further embodiment and in accordance with any of the above, the second variant IgG Fc domain is selected from a group including: (i) a second variant IgG1 Fc domain, (ii) a second variant IgG2 Fc domain, and (iii) a second variant IgG4 Fc domain.

In another aspect, a nucleic acid composition comprising nucleic acids encoding the first monomer, the second monomer, the third monomer, and the fourth monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain, wherein (i) the first monomer and the second monomer comprise a first set of VH:VL electrostatic variants comprising amino acid substitutions Q39E:Q38K, and a set of CH1:CL electrostatic variants comprising amino acid substitutions K213E/K218D:D122K/E123K, (ii) the third monomer and the fourth monomer comprise a second set of VH:VL electrostatic variants comprising amino acid substitutions Q39K:Q38E, and a set of CH1:CL steric variants comprising amino acid substitutions A141F:F116A, (iii) numbering of the first set and second set of VH:VL electrostatic variants is according to Kabat numbering, and (iv) numbering of the set of CH1:CL electrostatic variants and the set of CH1:CL steric variants is according to EU numbering.

In another aspect, a nucleic acid composition comprising nucleic acids encoding the first monomer, the second monomer, the third monomer, and the fourth monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain, wherein (i) the first monomer and the second monomer comprise a first set of VH:VL electrostatic variants comprising amino acid substitutions Q39E:Q38K, and a set of CH1:CL electrostatic variants comprising amino acid substitutions K213E/K218D:D122K/E123K, (ii) the third monomer and the fourth monomer comprise a second set of VH:VL electrostatic variants comprising amino acid substitutions Q39K:Q38E, and a set of CH1:CL steric variants comprising amino acid substitutions A141F:F118A, (iii) numbering of the first set and second set of VH:VL electrostatic variants is according to Kabat numbering, and (iv) numbering of the set of CH1:CL electrostatic variants and the set of CH1:CL steric variants is according to EU numbering.

In another aspect, a nucleic acid composition comprising nucleic acids encoding the first monomer, the second monomer, the third monomer, and the fourth monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain, wherein (i) the first monomer and the second monomer comprise a first set of VH:VL electrostatic variants comprising amino acid substitutions Q39E:Q38K, and a set of CH1:CL electrostatic variants comprising amino acid substitutions K213E/K218D:D122K/E123K, (ii) the third monomer and the fourth monomer comprise a second set of VH:VL electrostatic variants comprising amino acid substitutions Q39K:Q38E, and a set of CH1:CL steric variants comprising amino acid substitutions K147S:S131K, (iii) numbering of the first set and second set of VH:VL electrostatic variants is according to Kabat numbering, and (iv) numbering of the set of CH1:CL electrostatic variants and the set of CH1:CL steric variants is according to EU numbering.

In another aspect, a nucleic acid composition comprising nucleic acids encoding the first monomer, the second monomer, the third monomer, and the fourth monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a first monomer comprising a VH-CH1 domain, wherein VH is a variable heavy domain; and (b) a second monomer comprising a VL-CL domain, wherein VL is a variable light domain, wherein: (i) the variable heavy domain and the variable light domain form an antigen binding domain, and (ii) the first monomer and the second monomer comprise a set of CH1:CL electrostatic variants comprising amino acid substitutions at amino acid residues K213/K218:D122/E123, wherein numbering is according to EU numbering.

In a further embodiment and in accordance with the above, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering.

In a further embodiment and in accordance with any of the above, the set of CH1:CL electrostatic variants comprises amino acid substitutions K213E/K218D:D122K/E123K, wherein numbering is according to EU numbering.

In a further embodiment and in accordance with any of the above, further comprising a set of VH:VL electrostatic variants, wherein the set of VH:VL electrostatic variants comprises amino acid substitutions at amino acid residues Q39:Q38, wherein numbering is according to Kabat numbering.

In a further embodiment and in accordance with the above, the set of VH:VL electrostatic variants comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering.

In a further embodiment and in accordance with any of the above, the set of VH:VL electrostatic variants comprise amino acid substitutions Q39E:Q38K, wherein numbering is according to Kabat numbering.

In a further embodiment and in accordance with any of the above, further comprising a set of CH1:CL steric variants, wherein the set of CH1:CL steric variants comprises amino acid substitutions at amino acid residues selected from a group including: (i) A141:F118, (ii) A141:F116, and (iii) K147:S131, wherein numbering is according to EU numbering.

In a further embodiment and in accordance with the above, the set of CH1:CL steric variants comprises amino acid substitutions selected from a group including: (i) A141F:F118A, (ii) A141F:F116A, and (iii) K147S:S131K, wherein numbering is according to EU numbering.

In another aspect, a nucleic acid composition comprising nucleic acids encoding the first monomer and the second monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a first monomer comprising a VH-CH1 domain, wherein VH is a variable heavy domain; and (b) a second monomer comprising a VL-CL domain, wherein VL is a variable light domain, wherein: (i) the variable heavy domain and the variable light domain form an antigen binding domain, and (ii) the first monomer and the second monomer comprise a set of CH1:CL steric variants comprising amino acid substitutions at amino acid residues selected from a group including: (i) A141:F118, (ii) A141:F116, and (iii) K147:S131, wherein numbering is according to EU numbering.

In a further embodiment and in accordance with the above, the set of CH1:CL steric variants comprises amino acid substitutions selected from a group including: (i) A141F:F118A, (ii) A141F:F116A, and (iii) K147S:S131K, wherein numbering is according to EU numbering.

In a further embodiment and in accordance with any of the above, further comprising a set of VH:VL electrostatic variants, wherein the set of VH:VL electrostatic variants comprises amino acid substitutions at amino acid residues Q39:Q38, wherein numbering is according to Kabat numbering.

In a further embodiment and in accordance with the above, the set of VH:VL electrostatic variants comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering.

In a further embodiment and in accordance with any of the above, the set of VH:VL electrostatic variants comprise amino acid substitutions Q39E:Q38K, wherein numbering is according to Kabat numbering.

In a further embodiment and in accordance with any of the above, further comprising a set of CH1:CL electrostatic variants, wherein the set of CH1:CL electrostatic variants comprises amino acid substitutions at amino acid residues K213/K218:D122/E123, wherein numbering is according to EU numbering.

In a further embodiment and in accordance with the above, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering.

In a further embodiment and in accordance with any of the above, the set of CH1:CL electrostatic variants comprises amino acid substitutions K213E/K218D:D122K/E123K, wherein numbering is according to EU numbering.

In another aspect, a nucleic acid composition comprising nucleic acids encoding the first monomer and the second monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a first monomer comprising a VH-CH1 domain, wherein VH is a variable heavy domain; and (b) a second monomer comprising a VL-CL domain, wherein VL is a variable light domain, wherein: (i) the variable heavy domain and the variable light domain form an antigen binding domain, and (ii) the first monomer and the second monomer comprise a set of VH:VL electrostatic variants comprising amino acid substitutions at amino acid residues Q39:Q38, wherein numbering is according to Kabat numbering.

In a further embodiment and in accordance with the above, the set of VH:VL electrostatic variants comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering.

In a further embodiment and in accordance with any of the above, the set of VH:VL electrostatic variants comprise amino acid substitutions Q39E:Q38K, wherein numbering is according to Kabat numbering.

In a further embodiment and in accordance with any of the above, further comprising a set of CH1:CL electrostatic variants, wherein the set of CH1:CL electrostatic variants comprises amino acid substitutions at amino acid residues K213/K218:D122/E123, wherein numbering is according to EU numbering.

In a further embodiment and in accordance with the above, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering.

In a further embodiment and in accordance with any of the above, the set of CH1:CL electrostatic variants comprises amino acid substitutions K213E/K218D:D122K/E123K, wherein numbering is according to EU numbering.

In a further embodiment and in accordance with any of the above, further comprising a set of CH1:CL steric variants, wherein the set of CH1:CL steric variants comprises amino acid substitutions at amino acid residues selected from a group including: (i) A141:F118, (ii) A141:F116, and (iii) K147:S131, wherein numbering is according to EU numbering.

In a further embodiment and in accordance with the above, the set of CH1:CL steric variants comprises amino acid substitutions selected from a group including: (i) A141F:F118A, (ii) A141F:F116A, and (iii) K147S:S131K, wherein numbering is according to EU numbering.

In another aspect, a nucleic acid composition comprising nucleic acids encoding the first monomer and the second monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain, wherein: (1) the first monomer and the second monomer comprise a set of CH1:CL electrostatic variants and the third monomer and the fourth monomer comprise a set of CH1:CL steric variants, or the first monomer and the second monomer comprise a set of CH1:CL steric variants and the third monomer and the fourth monomer comprise a set of CH1:CL electrostatic variants, (2) the set of CH1:CL electrostatic variants comprises a set of amino acid substitutions selected from the group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, (3) the set of CH1:CL steric variants comprises a set of amino acid substitutions selected from the group including: (i) A141F:F118A, (ii) A141F:F116A, and (iii) K147S:S131K, (4) the first monomer and the second monomer comprise a first set of VH:VL electrostatic variants, and the third monomer and the fourth monomer comprise a second set of VH:VL electrostatic variants, (5) the first set of VH:VL electrostatic variants and the second set of VH:VL electrostatic variants comprise amino acid substitutions selected from the group including: (i) Q39E:Q38K and Q39K:Q38E, respectively, (ii) Q39K:Q38E and Q39E:Q38K, respectively, (iii) Q39E:Q38K and Q39R:Q38E, respectively, (iv) Q39E:Q38K and Q39R:Q38D, respectively, (v) Q39E:Q38K and Q39E:Q38K, respectively, (vi) Q39E:Q38K and Q39E:Q38R, respectively, (vii) Q39E:Q38K and Q39D:Q38K, respectively, (viii) Q39E:Q38K and Q39D:Q38R, respectively, (ix) Q39E:Q38R and Q39K:Q38E, respectively, (x) Q39E:Q38R and Q39K:Q38D, respectively, (xi) Q39E:Q38R and Q39R:Q38E, respectively, (xii) Q39E:Q38R and Q39R:Q38D, respectively, (xiii) Q39E:Q38R and Q39E:Q38K, respectively, (xiv) Q39E:Q38R and Q39E:Q38R, respectively, (xv) Q39E:Q38R and Q39D:Q38K, respectively, (xvi) Q39E:Q38R and Q39D:Q38R, respectively, (xvii) Q39D:Q38K and Q39K:Q38E, respectively, (xviii) Q39D:Q38K and Q39K:Q38D, respectively, (xix) Q39D:Q38K and Q39R:Q38E, respectively, (xx) Q39D:Q38K and Q39R:Q38D, respectively, (xxi) Q39D:Q38K and Q39E:Q38K, respectively, (xxii) Q39D:Q38K and Q39E:Q38R, respectively, (xxiii) Q39D:Q38K and Q39D:Q38K, respectively, (xxiv) Q39D:Q38K and Q39D:Q38R, respectively, (xxv) Q39D:Q38R and Q39K:Q38E, respectively, (xxvi) Q39D:Q38R and Q39K:Q38D, respectively, (xxvii) Q39D:Q38R and Q39R:Q38E, respectively, (xxviii) Q39D:Q38R and Q39R:Q38D, respectively, (xxix) Q39D:Q38R and Q39E:Q38K, respectively, (xxx) Q39D:Q38R and Q39E:Q38R, respectively, (xxxi) Q39D:Q38R and Q39D:Q38K, respectively, (xxxii) Q39D:Q38R and Q39D:Q38R, respectively, (xxxiii) Q39K:Q38E and Q39K:Q38E, respectively, (xxxiv) Q39K:Q38E and Q39K:Q38D, respectively, (xxxv) Q39K:Q38E and Q39R:Q38E, respectively, (xxxvi) Q39K:Q38E and Q39R:Q38D, respectively, (xxxvii) Q39E:Q38K and Q39K:Q38D, respectively, (xxxviii) Q39K:Q38E and Q39E:Q38R, respectively, (xxxix) Q39K:Q38E and Q39D:Q38K, respectively, (xl) Q39K:Q38E and Q39D:Q38R, respectively, (xli) Q39R:Q38E and Q39K:Q38E, respectively, (xlii) Q39R:Q38E and Q39K:Q38D, respectively, (xliii) Q39R:Q38E and Q39R:Q38E, respectively, (xliv) Q39R:Q38E and Q39R:Q38D, respectively, (xlv) Q39R:Q38E and Q39E:Q38K, respectively, (xlvi) Q39R:Q38E and Q39E:Q38R, respectively, (xlvii) Q39R:Q38E and Q39D:Q38K, respectively, (xlviii) Q39R:Q38E and Q39D:Q38R, respectively, (xlix) Q39K:Q38D and Q39K:Q38E, respectively, (1) Q39K:Q38D and Q39K:Q38D, respectively, (li) Q39K:Q38D and Q39R:Q38E, respectively, (lii) Q39K:Q38D and Q39R:Q38D, respectively, (liii) Q39K:Q38D and Q39E:Q38K, respectively, (liv) Q39K:Q38D and Q39E:Q38R, respectively, (lv) Q39K:Q38D and Q39D:Q38K, respectively, (lvi) Q39K:Q38D and Q39D:Q38R, respectively, (lvii) Q39R:Q38D and Q39K:Q38E, respectively, (lviii) Q39R:Q38D and Q39K:Q38D, respectively, (lix) Q39R:Q38D and Q39R:Q38E, respectively, (lx) Q39R:Q38D and Q39R:Q38D, respectively, (lxi) Q39R:Q38D and Q39E:Q38K, respectively, (lxii) Q39R:Q38D and Q39E:Q38R, respectively, (lxiii) Q39R:Q38D and Q39D:Q38K, respectively, and (lxiv) Q39R:Q38D and Q39D:Q38R, respectively, and (6) numbering of the set of CH1:CL electrostatic variants, numbering of the set of CH1:CL steric variants, numbering of the first set of VH:VL electrostatic variants, and numbering of the second set of VH:VL electrostatic variants are all according to EU numbering.

In a further embodiment and in accordance with the above, the set of CH1:CL electrostatic variants comprises amino acid substitutions K213E/K218D:D122K/E123K.

In a further embodiment and in accordance with any of the above, the set of CH1:CL electrostatic variants comprises amino acid substitutions A141F:F118A.

In a further embodiment and in accordance with any of the above, the first set of VH:VL electrostatic variants comprises amino acid substitutions Q39E:Q38K, and the second set of VH:VL electrostatic variants comprises amino acid substitutions Q39K:Q38E. In other embodiments, the first set of VH:VL electrostatic variants comprises amino acid substitutions Q39K:Q38E, and the second set of VH:VL electrostatic variants comprises amino acid substitutions Q39E:Q38K.

In a further embodiment and in accordance with any of the above, the first and/or second variant IgG Fc domains comprise one or more FcγRIIIA (CD16a) binding variant substitutions.

In a further embodiment and in accordance with the above, the one or more FcγRIIIA (CD16a) binding variant substitutions are selected from the group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering. In some further embodiments, the first and second variant IgG Fc domains comprise a set of FcγRIIIA (CD16a) binding variant substitutions selected from the group including: (i) S239D/I332E:S239D/I332E, (ii) S239D:S239D, (iii) 1332E:1332E, (iv) WT:S239D/I332E, (v) WT:S239D, (vi) WT:1332E, (vii) S239D/I332E:WT, (viii) S239D:WT, (ix) 1332E:WT, (x) S239D/I332E:S239D, (xi) S239D/I332E:1332E, (xii) S239D:S239D/I332E, (xiii) 1332E: S239D/I332E, (xiv) S239D:1332E, and (xv) 1332E:S239D, wherein numbering is according to EU numbering.

In a further embodiment and in accordance with any of the above, the first and/or second variant IgG Fc domains comprise the FcγRIIIA (CD16a) binding variant substitutions of S239D/I332E, wherein numbering is according to EU numbering.

In a further embodiment and in accordance with any of the above, the first and second variant IgG Fc domains comprise a set of heterodimerization variants selected from the group including those depicted in FIGS. 13A-13F, wherein numbering is according to EU numbering.

In a further embodiment and in accordance with the above, the set of heterodimerization variants is selected from the group including: (i) S364K/E357Q:L368D/K370S, (ii) S364K: L368D/K370S, (iii) S364K:L368E/K370S, (iv) D401K:T411E/K360E/Q362E, and (v) T366W: T366S/L368A/Y407V, wherein numbering is according to EU numbering.

In a further embodiment and in accordance with any of the above, the first and second variant IgG Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant IgG Fc domains comprise one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first variant IgG Fc domain comprises amino acid variants S364K/E357Q/E233P/L234V/L235A/G236de1/S267K, wherein the second variant IgG Fc domain comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second variant IgG Fc domains each further comprise amino acid variants M428L/N434S or M428L/N434A, wherein numbering is according to EU numbering.

In a further embodiment and in accordance with any of the above, the first variant IgG Fc domain is selected from the group including: (i) a first variant IgG1 Fc domain, (ii) a first variant IgG2 Fc domain, and (iii) a first variant IgG4 Fc domain.

In a further embodiment and in accordance with any of the above, the second variant IgG Fc domain is selected from the group including: (i) a second variant IgG1 Fc domain, (ii) a second variant IgG2 Fc domain, and (iii) a second variant IgG4 Fc domain.

In another aspect, a nucleic acid composition comprising nucleic acids encoding the first monomer, the second monomer, the third monomer, and the fourth monomer of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect, the present disclosure provides a multimeric protein, comprising: (a) a variant human IgG1 CH1 domain (vCH1); and (b) a variant human kappa CL domain (vCL), wherein said CH1 domain and said CL domain together have a set of amino acid substitutions selected from the group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering.

In another aspect, a nucleic acid composition comprising nucleic acids encoding the variant human IgG1 CH1 domain and the variant human kappa CL domain of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

In another aspect the present disclosure provides a multimeric protein, comprising: (a) a variant human IgG1 CH1 domain (vCH1); and (b) a variant human kappa CL domain (vCL), wherein: (1) said CH1 domain and said CL domain together have a first set of amino acid substitutions selected from the group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, (2) said CH1 domain and said CL domain together have a second set of amino acid substitutions selected from the group including: (i) A141F:F118A, (ii) A141F:F116A, and (iii) K147S:S131K, and (3) numbering of said first set of amino acid substitutions and numbering of said second set of amino acid substitutions are both according to EU numbering.

In another aspect, a nucleic acid composition comprising nucleic acids encoding the variant human IgG1 CH1 domain and the variant human kappa CL domain of the multimeric protein (as described above) is provided.

In another aspect, an expression vector comprising the nucleic acids (as described above) is provided.

In another aspect, a host cell transformed with an expression vector (as described above) is provided.

In another aspect, a method of making a multimeric protein is provided, the method comprising: (a) culturing the host cell (as described above) under conditions wherein the multimeric protein is expressed; and (b) recovering the multimeric protein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Fig.,” “FIG.,” “Figure,” “Figures,” “Figs.,” and “FIGs.” herein) of which:

FIG. 1 depicts that when producing bispecific antibodies in IgG format, there are potentially nine incorrect pairings.

FIG. 2 depicts schematics showing (FIG. 2A) correct Fab pairings and (FIG. 2B) incorrect Fab pairings resulting from introduction of electrostatic variants at K213/K218:D122/E123 in the CH1:CL interface.

FIG. 3 depicts electrostatic variants which may be introduced at K213/K218:D122/E123 in the CH1:CL interface.

FIG. 4 depicts schematics showing (FIG. 4A) correct Fab pairings and (FIG. 4B) incorrect Fab pairings resulting from introduction of steric variants at A141:F118 in the CH1:CL interface.

FIG. 5 depicts electrostatic variants which may be introduced at A141:F116, A141:F118, or K147:S131 in the CH1:CL interface.

FIG. 6 depicts schematics showing (FIG. 6A) correct Fab pairings and (FIG. 6B) incorrect Fab pairings resulting from introduction of electrostatic variants at Q39:Q38 in the VH:VL interface.

FIG. 7 depicts electrostatic variants (FIGS. 7A and 7B) which may be introduced at Q39:Q38 in the VH:VL interface.

FIG. 8 depicts schematics showing (FIG. 8A) correct Fab pairings and (FIG. 8B) incorrect Fab pairings resulting from Q39E:Q38K in VH:VL interface and K213E/K218D:D122K/E123K in CH1:CL interface of Fab A, and Q39K:Q38E in VH:VL interface and A141F:F118A interface of Fab B.

FIG. 9 (FIGS. 9A-9C) depicts sequences of illustrative multimeric proteins which include Q39E:Q38K in VH:VL interface and K213E/K218D:D122K/E123K in CH1:CL interface of Fab A, and Q39K:Q38E in VH:VL interface and A141F:F116A, A141F:F118A, or K147S:S131K in the CH1:CL interface of Fab B.

FIG. 10 depicts the separation of species from protein A purified XENP40711 by (FIG. 10A) analytical size-exclusion chromatography (aSEC), (FIG. 10B) analytical cation-exchange chromatography (aCEC), and (FIG. 10C) analytical anion-exchange chromatography (aAEC).

FIG. 11 depicts the separation of species from protein A+AEC purified XENP40711 by (FIG. 11A) aSEC, (FIG. 11B) aCEC, and (FIG. 11C) aAEC.

FIG. 12 depicts the separation of species from protein A+AEC+CEC purified XENP40711 by (FIG. 12A) aSEC, (FIG. 12B) aCEC, and (FIG. 12C) aAEC.

FIG. 13 (FIGS. 13A-13F) depicts useful pairs of Fc heterodimerization variant sets (including skew and pI variants). In FIG. 13F, there are variants for which there are no corresponding “monomer 2” variants. Such variants are pI variants that can be used alone on either monomer of a multimeric protein. Heterodimer yield (%) and CH3 Tₘ (° C.) of preferred Fc heterodimerization variants were previously described (see, e.g., FIG. 8 of U.S. Patent Publication No. 2019/0248898).

FIG. 14 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 15 depicts useful ablation variants that ablate FcTR binding (also referred to as “knockouts” or “KO” variants). In some embodiments, such ablation variants are included in the Fc domain of both monomers of the subject multimeric protein described herein. In other embodiments, the ablation variants are only included on only one variant Fc domain.

FIG. 16 depicts useful Fc variants that increase FcγRIIIA binding and enhance ADCC activity. These variants may be used with or without Xtend variants (M428L/N434S or M428L/N434A).

FIG. 17 shows a particularly useful embodiment of the heterodimeric Fc domains (i.e., CH2-CH3 in this embodiment) of the multimeric proteins of the invention.

FIG. 18 (FIGS. 18A-18D) shows the sequences of several useful bispecific IgG1 backbones based on human IgG1, without the variable domains. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains. Heterodimeric Fc backbone 13 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 14 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (G446_) and/or lysine (K447_). The C-terminal glycine and/or lysine deletion may be intentionally engineered to reduce heterogeneity. Additionally, C-terminal glycine and/or lysine deletion may occur naturally for example during production and storage. Additionally, these sequences may include the H435R/Y436F variant in either of monomer 1 or monomer 2 for facile purification.

FIG. 19 (FIGS. 19A-19C) shows the sequences of several useful bispecific IgG1 backbones based on human IgG1 and having WT effector function. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and N297S variant that removes glycosylation on both chains. Heterodimeric Fc backbone 8 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434S Xtend variants on both chains. Heterodimeric Fc backbone 9 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 10 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434A Xtend variants on both chains. Heterodimeric Fc backbone 11 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

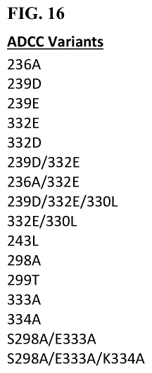

FIG. 20 (FIGS. 20A-20C) depicts the sequences of several useful bispecific IgG1 backbones based on human IgG1 and having enhanced ADCC function. The sequences here are based on heterodimeric Fc backbone 1 in FIG. 19, although the ADCC variants in FIG. 16 may also be included in any of the other heterodimeric Fc backbones in FIG. 19. ADCC-enhanced Heterodimeric Backbone 1 includes S239D/I332E on both the first and the second heterodimeric Fc chain. ADCC-enhanced Heterodimeric Backbone 2 includes S239D on both the first and the second heterodimeric Fc chain. ADCC-enhanced Heterodimeric Backbone 3 includes 1332E on both the first and the second heterodimeric Fc chain. ADCC-enhanced Heterodimeric Backbone 4 includes S239D/I332E on the second heterodimeric Fc chain. ADCC-enhanced Heterodimeric Backbone 5 includes S239D on the second heterodimeric Fc chain.

ADCC-enhanced Heterodimeric Backbone 6 includes 1332E on the second heterodimeric Fc chain. ADCC-enhanced Heterodimeric Backbone 7 includes S239D/I332E on the first heterodimeric Fc chain. ADCC-enhanced Heterodimeric Backbone 8 includes S239D on the first heterodimeric Fc chain. ADCC-enhanced Heterodimeric Backbone 9 includes 1332E on the first heterodimeric Fc chain. ADCC-enhanced Heterodimeric Backbone 10 includes S239D/I332E on the first heterodimeric Fc chain and S239D on the second heterodimeric Fc chain. ADCC-enhanced Heterodimeric Backbone 11 includes S239D/I332E on the first heterodimeric Fc chain and 1332E on the second heterodimeric Fc chain. ADCC-enhanced Heterodimeric Backbone 12 includes S239D on the first heterodimeric Fc chain and S239D/I332E on the second heterodimeric Fc chain. ADCC-enhanced Heterodimeric Backbone 13 includes 1332E on the first heterodimeric Fc chain and S239D/I332E on the second heterodimeric Fc chain. ADCC-enhanced Heterodimeric Backbone 14 includes S239D on the first heterodimeric Fc chain and 1332E on the second heterodimeric Fc chain. ADCC-enhanced Heterodimeric Backbone 15 includes 1332E on the first heterodimeric Fc chain and S239D on the second heterodimeric Fc chain.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1. That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIG. 21 (FIGS. 21A-21C) shows the sequences of several useful bispecific IgG1 backbones based on human IgG1 and having enhanced ADCC function and enhanced serum half-life. The sequences here are based on heterodimeric Fc backbone 8 in FIG. 19, although the ADCC variants in FIG. 16 may also be included in any of the other heterodimeric Fc backbones in FIG. 19. ADCC-enhanced Heterodimeric Backbone 1 with Xtend includes S239D/I332E on both the first and the second heterodimeric Fc chain. ADCC-enhanced Heterodimeric Backbone 2 with Xtend includes S239D on both the first and the second heterodimeric Fc chain. ADCC-enhanced Heterodimeric Backbone 3 with Xtend includes 1332E on both the first and the second heterodimeric Fc chain. ADCC-enhanced Heterodimeric Backbone 4 with Xtend includes S239D/I332E on the second heterodimeric Fc chain. ADCC-enhanced Heterodimeric Backbone 5 with Xtend includes S239D on the second heterodimeric Fc chain. ADCC-enhanced Heterodimeric Backbone 6 with Xtend includes 1332E on the second heterodimeric Fc chain. ADCC-enhanced Heterodimeric Backbone 7 with Xtend includes S239D/I332E on the first heterodimeric Fc chain. ADCC-enhanced Heterodimeric Backbone 8 with Xtend includes S239D on the first heterodimeric Fc chain. ADCC-enhanced Heterodimeric Backbone 9 with Xtend includes 1332E on the first heterodimeric Fc chain. ADCC-enhanced Heterodimeric Backbone 10 with Xtend includes S239D/I332E on the first heterodimeric Fc chain and S239D on the second heterodimeric Fc chain. ADCC-enhanced Heterodimeric Backbone 11 with Xtend includes S239D/I332E on the first heterodimeric Fc chain and 1332E on the second heterodimeric Fc chain. ADCC-enhanced Heterodimeric Backbone 12 with Xtend includes S239D on the first heterodimeric Fc chain and S239D/I332E on the second heterodimeric Fc chain. ADCC-enhanced Heterodimeric Backbone 13 with Xtend includes 1332E on the first heterodimeric Fc chain and S239D/I332E on the second heterodimeric Fc chain. ADCC-enhanced Heterodimeric Backbone 14 with Xtend includes S239D on the first heterodimeric Fc chain and 1332E on the second heterodimeric Fc chain. ADCC-enhanced Heterodimeric Backbone 15 with Xtend includes 1332E on the first heterodimeric Fc chain and S239D on the second heterodimeric Fc chain.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1. That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIG. 22 (FIGS. 22A-22C) shows the sequences of several useful bispecific IgG1 backbones based on human IgG1 and having enhanced ADCC function and alternate enhanced serum half-life variants 428L/434A. The sequences here are based on heterodimeric Fc backbone 8 in FIG. 20, although the ADCC variants in FIG. 16 may also be included in any of the other heterodimeric Fc backbones in FIG. 20. ADCC-enhanced Heterodimeric Backbone 1 with Xtend includes S239D/I332E on both the first and the second heterodimeric Fc chain. ADCC-enhanced Heterodimeric Backbone 2 with Xtend includes S239D on both the first and the second heterodimeric Fc chain. ADCC-enhanced Heterodimeric Backbone 3 with Xtend includes 1332E on both the first and the second heterodimeric Fc chain. ADCC-enhanced Heterodimeric Backbone 4 with Xtend includes S239D/I332E on the second heterodimeric Fc chain. ADCC-enhanced Heterodimeric Backbone 5 with Xtend includes S239D on the second heterodimeric Fc chain. ADCC-enhanced Heterodimeric Backbone 6 with Xtend includes 1332E on the second heterodimeric Fc chain. ADCC-enhanced Heterodimeric Backbone 7 with Xtend includes S239D/I332E on the first heterodimeric Fc chain. ADCC-enhanced Heterodimeric Backbone 8 with Xtend includes S239D on the first heterodimeric Fc chain. ADCC-enhanced Heterodimeric Backbone 9 with Xtend includes 1332E on the first heterodimeric Fc chain. ADCC-enhanced Heterodimeric Backbone 10 with Xtend includes S239D/I332E on the first heterodimeric Fc chain and S239D on the second heterodimeric Fc chain. ADCC-enhanced Heterodimeric Backbone 11 with Xtend includes S239D/I332E on the first heterodimeric Fc chain and 1332E on the second heterodimeric Fc chain. ADCC-enhanced Heterodimeric Backbone 12 with Xtend includes S239D on the first heterodimeric Fc chain and S239D/I332E on the second heterodimeric Fc chain. ADCC-enhanced Heterodimeric Backbone 13 with Xtend includes 1332E on the first heterodimeric Fc chain and S239D/I332E on the second heterodimeric Fc chain. ADCC-enhanced Heterodimeric Backbone 14 with Xtend includes S239D on the first heterodimeric Fc chain and 1332E on the second heterodimeric Fc chain. ADCC-enhanced Heterodimeric Backbone 15 with Xtend includes 1332E on the first heterodimeric Fc chain and S239D on the second heterodimeric Fc chain.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1. That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIG. 23 depicts sequences for “CH1” that find use in embodiments of the multimeric proteins of the invention. Although some of these CH1 sequences include N208D substitution, each of these sequences may be considered as “WT” CH1 backbone on which the CH1 electrostatic and steric variants of the invention may be engineered.

FIG. 24 depicts sequences for “hinge” that find use in embodiments of multimeric proteins of the invention.

FIG. 25 depicts the “WT” constant domain of the cognate light chains that find use in the subject multimeric proteins of the invention on which the CL electrostatic and steric variants of the invention may be engineered.

FIG. 26 depicts a number of exemplary domain linkers. In some embodiments, these linkers find use linking a single-chain Fv to an Fc chain. In some embodiments, these linkers may be combined. For example, a (G)₄S (SEQ ID NO: 166) linker may be combined with a “half hinge” linker.

FIG. 27 depicts the serum concentration of XENP40711 overtime in 3 individual cynomolgus monkeys.

### DETAILED DESCRIPTION OF THE INVENTION

The description is presented to enable one of ordinary skill in the art to make and use the invention and is provided in the context of a patent application and its requirements. The section headings used herein are for organization purposes only and are not to be construed as limiting the subject matter described. While various embodiments of the invention(s) of the present disclosure have been shown and described herein, it will be obvious to those skilled in the art that such embodiments are provided by way of example only. Numerous variations, changes, and substitutions may occur to those skilled in the art without departing from the invention(s). It should be understood that various alternatives to the embodiments of the invention(s) described herein may be employed in practicing any one of the inventions(s) set forth herein.

All patents, published patent applications, other publications, and sequences from GenBank, and other databases referred to herein are incorporated by reference in their entirety with respect to the related technology.

### I. Overview

Production of multimeric proteins that include two heavy chains and two light chains is challenging, as heavy chains bind light chains in a relatively promiscuous manner. The invention provides methods for (1) promoting the proper pairing of multimeric protein monomers, and/or (2) dissuading the improper pairing of multimeric protein monomers, through the use of interface variants.

### II. Definitions

In order that the application may be more completely understood, several definitions are set forth below. Such definitions are meant to encompass grammatical equivalents.

As used herein, a “protein domain” is a structural and/or functional unit of a protein. In general, individual protein domains are self-stabilizing and self-assembling (including when a domain is made up of two or more polypeptide chains). Exemplary protein domains in the context of the invention include, but are not limited to, variable heavy (VH) domains, variable light (VL) domains, Fv domains, scFv domains, Fab domains, CH1 domains, CL domains, hinge domains, CH2 domains, CH3 domains, (IgG) Fc domains, light chain domains and heavy chain domains. As will be appreciated by those in the art, smaller domains maybe part of larger domains; e.g., VH and VL domains together with a scFv linker can form a scFv domain; CH2 and CH3 domains form an (IgG) Fc domain (with or without the hinge domain); VH, CH1, hinge, CH2 and CH3 domains together form a heavy chain domain, etc.

By “multimeric protein” herein is meant a protein complex comprising two or more protein domains. As will be appreciated by those in the art, the protein domains of a multimeric proteins can be covalently and/or non-covalently attached in a variety of ways. In some cases, the multimeric protein is expressed as a single polypeptide chain, e.g., the domains are covalently attached using peptide bonds and are expressed as a unit. In the case where two protein domains that are not normally associated are combined with optional domain linkers this is also sometimes referred to as a “fusion protein.” As will be appreciated by those in the art, after expression, there can be additional covalent bonds that are generated, for example disulfide bonds that form between different parts of the protein. In some cases, the multimeric protein comprises more than one polypeptide chain, and these polypeptide sequences can be either be covalently or non-covalently associated, or both. For example, a traditional tetrameric antibody has four polypeptide chains (two heavy chains and two light chains) that are expressed as different polypeptide chains and then form disulfides between the different protein domains. Additionally, it should be understood that some multimeric proteins include smaller multimeric domains. For example, a variable heavy domain and a variable light domain can be attached with a linker domain to form a scFv, which is a multimeric protein. Then the scFv can be included in a larger multimeric protein such as those described herein. Furthermore, the multimeric proteins can be homomultimers or heteromultimers. That is, a multimeric protein may comprise two or more identical polypeptide chains while not containing any different polypeptide chains (i.e., a “homomultimeric” protein). A “homomultimer” consists of two or more copies of the same polypeptide chain. Similarly, a “homodimer” consists of two copies of the same polypeptide chain, a “homotrimer” consists of three copies of the same polypeptide chain, etc. Alternatively, a multimeric protein may comprise at least two different polypeptide chains (i.e., a “heteromultimeric” protein). If the “heteromultimer” has three or more polypeptide chains, some of them can be identical to each other as long as at least one is different from the others. Generally, a “dimer” refers to a multimeric protein with two different polypeptide chains, even if they later become covalently attached through disulfide bonds, for example. A trimer has three different polypeptide chains, etc.

As used herein, the term “CH1:CL interface variants” refers to a “set” of amino acid substitutions that (1) promotes the proper pairing of multimeric protein monomers wherein at least one of the monomers includes a CH1 domain and at least one of the other monomers includes a CL domain, and/or (2) dissuades the improper pairing of multimeric protein monomers wherein at least one of the monomers includes a CH1 domain and at least one of the other monomers includes a CL domain. Generally, the CH1:CL interface variants promote the proper pairing/dissuade the improper pairing through select mechanisms, as described below. The term “CH1:CL interface electrostatic variants” (or “CH1:CL electrostatic variants”) refers to CH1:CL interface variants that promote the proper pairing of multimeric protein monomers through the formation of a salt bridge between the CH and CL domains, as well as through charge complementarity, and/or dissuade the improper pairing of multimeric protein monomers through electrostatic charge repelling. The term “CH1:CL interface steric variants” (or “CH1:CL steric variants”) refers to CH1:CL variants that promote the proper pairing of multimeric protein monomers (and/or dissuade the improper pairing of multimeric protein monomers) by virtue of steric complementarity. The CH1:CL interface variants described herein are set forth in an X:Y format, wherein X denotes one or more amino acid substitutions in the CH1 domain and Y denotes one or more amino acid substitutions in the CL domain of, for instance, a Fab. For example, the CH1:CL electrostatic variants K213/K218:D122/E123 refers to amino acid substitutions at residues K213 and K218 in the CH1 domain and amino acid substitutions at residues D122 and E123 in the CL domain.

As used herein, the term “VH:VL interface electrostatic variants” (or “VH:VL electrostatic variants”) refers to a “set” of amino acid substitutions that promotes the proper pairing of multimeric protein monomers and/or dissuades the improper pairing of multimeric protein monomers through the formation of a hydrogen bond pair between the VH and the VL domains of, for instance, a Fab. Similar to the CH1:CL interface variants, the VH:VL electrostatic variants are set forth in an X:Y format, wherein X denotes one or more amino acid substitutions in the VH domain and Y denotes one or more amino acid substitutions in the VL domain. For example, the VH:VL electrostatic variants Q39:Q38 refers to an amino acid substitution at residue Q39 in the VH domain and an amino acid substitution at residue Q38 in the VL domain.

As used herein, the term “set,” when discussed in the context of interface variants, refers to a paired grouping of amino acid substitutions wherein a first subset of the paired grouping of amino acid substitutions are made with respect to a first monomer (e.g., CH1, VH, etc.) and a second subset of the paired grouping of amino acid substitutions are made with respect to a second monomer (e.g., CL, VL, etc.). In some instances, the paired grouping of amino acid substitutions can comprise one or more amino acid substitutions in CH1 and one or more amino acid substitutions in CL (i.e., in the context of CH1:CL interface variants). In other instances, the paired grouping of amino acid substitutions can comprise one or more amino acid substitutions in VH and one or more amino acid substitutions in VL (i.e., in the context of VH:VL electrostatic variants).

By “protein” as used herein is meant at least two covalently attached amino acids, which includes proteins, polypeptides, oligopeptides, and peptides. In addition, polypeptides that make up the multimeric proteins of the invention may include synthetic derivatization of one or more side chains or termini, glycosylation, PEGylation, circular permutation, cyclization, linkers to other molecules, fusion to proteins or protein domains, and addition of peptide tags or labels. By “residue” as used herein is meant a position in a protein and its associated amino acid identity. For example, Asparagine 297 (also referred to as Asn297 or N297) is a residue at position 297 in the human antibody IgG1.

By “amino acid” and “amino acid identity” as used herein is meant one of the 20 naturally occurring amino acids that are coded for by DNA and RNA.

As used herein, the term “antibody” is used generally. Multimeric proteins provided herein can take on a number of formats as described herein, including traditional antibodies as well as antibody derivatives, fragments, and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

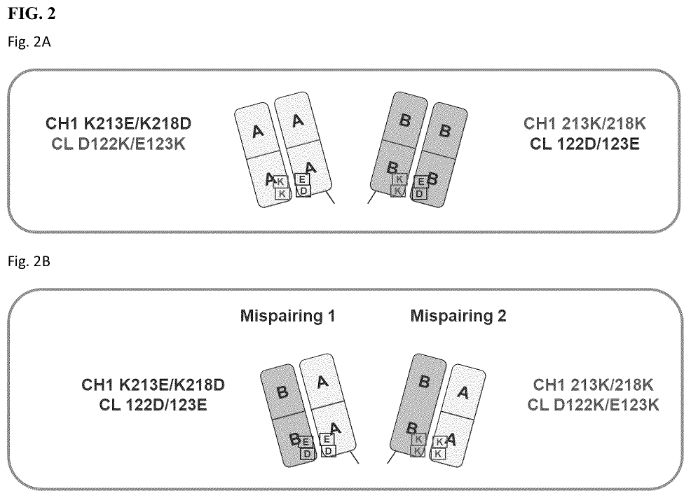

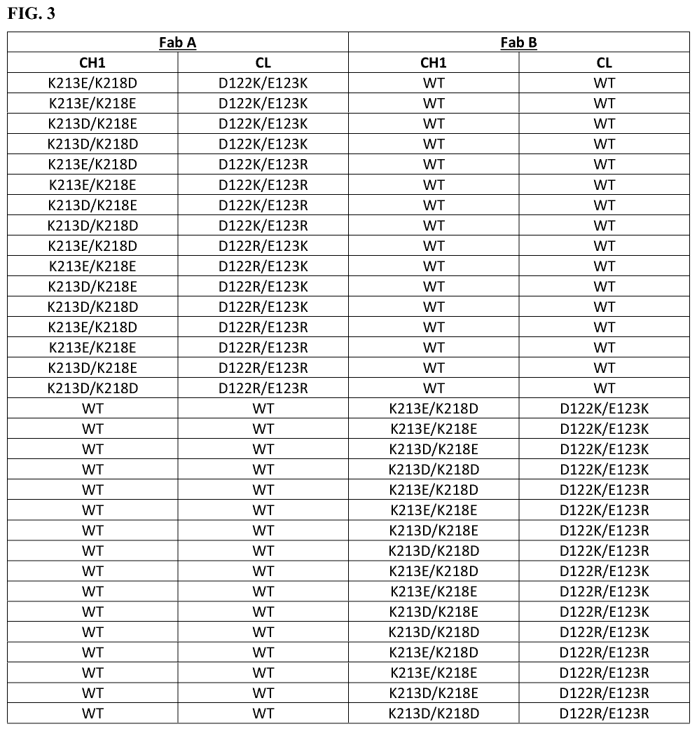

Other useful antibody formats include, but are not limited to, “scFv-mAb,” “mAb-Fv,” “mAb-scFv,” “central-Fv,” and “central-scFv2,” format antibodies, as disclosed in U.S. Pat. No. 10,793,632, which is incorporated by reference herein, particularly in pertinent part relating to antibody formats (see, e.g., FIG. 2 of U.S. Pat. No. 10,793,632).

Heavy chains of antibodies (as well as multimeric proteins) typically include a variable heavy (VH) domain, which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody (or multimeric protein), of which there are different categories or “isotypes,” such as, for example, IgG.

In some embodiments, the multimeric proteins provided herein include IgG isotype constant domains, which has several subclasses, including, but not limited to IgG1, IgG2, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic multimeric proteins can also comprise hybrids of isotypes and/or subclasses. For example, as shown in US Publication No. 2009/0163699, incorporated by reference, the present multimeric proteins, in some embodiments, include human IgG1/G2 hybrids.

By “Fc” or “Fc region” or “Fc domain” as used herein is meant the polypeptide comprising the constant region of an antibody (or multimeric protein), in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cδ2 and Cδ3), and optionally all or a portion of the hinge region between CH1 (Cδ1) and CH2 (Cδ2). Thus, in some cases, the Fc domain includes, from N- to C-terminal, CH2-CH3 and hinge-CH2-CH3. In some embodiments, the Fc domain is that from IgG1, IgG2, or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of a multimeric protein (or fragments thereof, such as, for example, an antibody (or fragments thereof)), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody (or multimeric protein. Structurally, the IgG CH1 domain ends at EU position 215, and the IgG CH2 domain begins at residue EU position 231. Thus, for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the multimeric proteins herein have at least one of the cysteines at position 220 according to EU numbering (hinge region) replaced by a serine. Generally, this modification is on the “scFv monomer” side (when 1+1 or 2+1 formats are used) for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5^{th} Ed., United States Public Health Service, National Institutes of Health, Bethesda, entirely incorporated by reference. In some embodiments, a subject multimeric protein comprises a substitution mutation at amino acid residue K218. While residue K218 can be considered to be a part of the hinge region according to EU numbering, it is expressly contemplated that, when discussed in the context of a CH1:CL interface variant (such as, for example, a CH1:CL electrostatic variant) or a set of CH1:CL interface variants (such as, for example, a set of CH1:CL electrostatic variants), residue K218 is considered to be a part of the CH1 domain, even in instances where amino acid substitutions of this residue are characterized as “according to EU numbering.”

TABLE 1
A.

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The light chain of an antibody (or multimeric protein) generally comprises two domains: the variable light domain (VL), which includes light chain CDRs vlCDR1-3, and a constant light chain region (often referred to as CL or C_{L} or CK). The antibody light chain is typically organized from N- to C-terminus: VL-CL.

By “antigen binding domain” or “ABD” herein is meant a set of six Complementary Determining Regions (CDRs) that, when present as part of a polypeptide sequence, specifically binds a target antigen. Non-limiting examples of a target antigen include: a tumor target antigen, a cancer target antigen, a T cell antigen, a NK cell antigen, a B cell antigen, an immune cell antigen, a non-cancer related target antigen, and the like. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27(1): 55-77 (2003).

TABLE 2

| | Kabat + Chothia | IMGT | Kabat | AbM | Chothia | Contact | Xencor |
|---|---|---|---|---|---|---|---|
| vhCDR1 | 26-35 | 27-38 | 31-35 | 26-35 | 23-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and non-conformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy and a variable light domain. In a “Fab” format, the set of 6 CDRs are contributed by two different polypeptide sequences, the variable heavy domain (vh or VH or V_{H}; containing the vhCDR1, vhCDR2 and vhCDR3) and the variable light domain (vl or VL or V_{L}; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of all or part of the hinge in the second monomer.

By “variable region” or “variable domain” as used herein is meant the region of an immunoglobulin that comprises one or more Ig domains substantially encoded by any of the V_{κ}, V_{λ}, and/or V_{H} genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g., H.X_L.Y means N- to C-terminal is VH-linker-VL, and L.Y_H.X is VL-linker-VH.

Some embodiments of the subject multimeric proteins provided herein can comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using vh and vl sequences from Fabs), to VL-scFv linker-VH, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ), E233_, or E233del designates a deletion of glutamic acid at position 233. Additionally, EDA233- or EDA233 #designates a deletion of the sequence GluAspAla that begins at position 233.

By “variant protein” or “protein variant,” or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild-type sequence, such as the heavy constant domain or Fc region from IgG1, IgG2, or IgG4, although human sequences with variants can also serve as “parent polypeptides,” for example the IgG1/2 hybrid of US Publication No. 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2, or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, or IgG4 (or hybrids thereof) is considered a non-naturally occurring modification.

By “effector function” as used herein is meant a biochemical event that results from the interaction of an antibody Fc region with an Fc receptor or ligand. Effector functions include but are not limited to ADCC, ADCP, and CDC.

By “ADCC” or “antibody dependent cell-mediated cytotoxicity” as used herein is meant the cell-mediated reaction, wherein nonspecific cytotoxic cells that express FcyRs recognize bound antibody on a target cell and subsequently cause lysis of the target cell. ADCC is correlated with binding to FcyRIIIa; increased binding to FcyRIIIa leads to an increase in ADCC activity.

By “ADCP” or antibody dependent cell-mediated phagocytosis as used herein is meant the cell-mediated reaction wherein nonspecific phagocytic cells that express FcyRs recognize bound antibody on a target cell and subsequently cause phagocytosis of the target cell.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcyR. Fc ligands also include Fc receptor homologs (FcRH), which are a family of Fc receptors that are homologous to the FcyRs (Davis et al., 2002, Immunological Reviews 190: 123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor,” “FcyR,” “FcγR,” or “FcgammaR” as used herein is meant any member of the family of proteins that bind the IgG antibody Fc region and is encoded by an FcyR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcyRIIb-1 and FcyRIIb-2), and FcγRIIc; and FcγRIII (CD16), including isoforms FcyRIIIa (including allotypes V158 and F158) and FcγRIIIb (including allotypes FcyRIIb-NA1 and FcyRIIb-NA2) (Jefferis et al., 2002, Immunol Lett 82:57-65, entirely incorporated by reference), as well as any undiscovered human FcyRs or FcyR isoforms or allotypes. An FcyR may be from any organism, including but not limited to humans, mice, rats, rabbits, and monkeys. Mouse FcyRs include but are not limited to FcγRI (CD64), FcγRII (CD32), FcγRIII (CD16), and FcγRIII-2 (CD16-2), as well as any undiscovered mouse FcyRs or FcyR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “target antigen” as used herein is meant the molecule that is bound specifically by the antigen binding domain comprising the variable regions of a given multimeric protein (or antibody).

By “strandedness” in the context of the monomers of the multimeric proteins of the invention herein is meant that, similar to the two strands of DNA that “match,” heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a multimeric protein (such as, for example, a bispecific antibody) according to the invention herein is meant a cell that contains the exogeneous nucleic acids encoding the components of the multimeric protein and is capable of expressing the multimeric protein under suitable conditions. Suitable host cells are discussed below.

By “wild-type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T.F. & Waterman, M.S. (1981) “Comparison Of Biosequences,” Adv. Appl. Math. 2:482 [local homology algorithm]; Needleman, S.B. & Wunsch, CD. (1970) “A General Method Applicable To The Search For Similarities In The Amino Acid Sequence Of Two Proteins,” J. Mol. Biol.48:443 [homology alignment algorithm], Pearson, W.R. & Lipman, D.J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see_s/.aIb.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters.

The multimeric proteins of the present invention are generally isolated or recombinant. “Isolated,” when used to describe the various polypeptides disclosed herein (such as, for example, multimeric proteins, antibodies, etc.), means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the multimeric proteins (or antibodies) are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody (or multimeric protein) having a K_{D} for an antigen or epitope of at least about 10˜4 M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where K_{D} refers to a dissociation rate of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a K_{A} or Kₐ for an antigen or epitope of at least 20-, 50-, 100-, 500-, 1000-, 5,000-, 10,000- or more times greater for the epitope relative to a control, where K_{A} or Kₐ refers to an association rate of a particular antibody-antigen interaction. Binding affinity is generally measured using a Biacore, SPR or BLI assay.

In some embodiments, binding affinity of a multimeric protein described herein is substantially equivalent to the binding affinity of a corresponding multimeric protein lacking interface variant(s). In some instances, binding affinity of a multimeric protein comprising one or more of the following interface variants: (i) a set of CH1:CL electrostatic variants, (ii) a set of CH1:CL steric variants, and (iii) one or more sets of VH:VL electrostatic variants, may have binding affinity substantially equivalent to a corresponding multimeric protein lacking the one or more interface variants. As used herein, the term “substantially equivalent” is meant to refer to two or more numeric values that exhibit a sufficiently high degree of similarity such that a person of ordinary skill in the art would consider the difference between the two or more numeric values to be biologically and/or statistically insignificant (such as, for example, a first and second binding affinity value). Binding affinity can be measured using a Biacore, SPR, or BLI assay.

By “ablation” herein is meant a decrease or removal of activity. Thus, for example, “ablating FcyR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay.

As used herein, the singular forms “a,” “an,” and “the” include plural referents unless the context clearly dictates otherwise. Thus, for example, reference to “an antigen” includes mixtures of antigens; reference to “a pharmaceutically acceptable carrier” includes mixtures of two or more such carriers, and the like. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A (alone),” and “B (alone).”

As used herein, the term “about” a value (or parameter) refers to, for example, ±1%, ±2%, ±3%, ±4%, +5%, 6%, ±7%, ±8%, ±9%, +10% and the like of a stated value. When referring to a range of values (or parameters), the term “about” refers to +10% of the upper limit and −10% of the lower limit of a stated range of values. When a range of values is provided, it is to be understood that each intervening value between the upper and lower limit of that range, and any other stated or intervening value in that stated range, is encompassed within the scope of the present disclosure. Where the stated range includes upper and/or lower limits, ranges excluding either of those included limits are also included in the present disclosure.

### III. Multimeric Proteins

The present invention is directed to the generation of multimeric proteins (generally, antibodies or fragments thereof), particularly bispecific binding proteins, in a manner that reduces the promiscuous pairing of monomers of the multimeric proteins and/or promotes the proper pairing of monomers of the multimeric proteins. Generation of the multimeric proteins generally relies on the introduction of amino acid substitutions into the monomers of the multimeric proteins, as described further herein.

### A. Tetrameric Proteins

In one aspect, the subject multimeric protein is a tetrameric protein (i.e., a multimeric protein comprising four monomers). In such instances, the term “monomer” is meant one quarter of the multimeric protein. In embodiments where the multimeric protein is a tetrameric protein, the subject multimeric protein generally comprises: (a) a first monomer, (b) a second monomer, (c) a third monomer, and (d) a fourth monomer.

In some embodiments, the first monomer comprises a VH1-CH1-hinge-CH2-CH3 monomer, wherein VH1 is a first variable heavy domain and CH2-CH3 is a first variant IgG Fc domain. In other words, the first monomer includes from N-terminus to C-terminus, a first variable heavy domain, a CH1 domain, a hinge domain, a CH2 domain, and a CH3 domain. In some embodiments, the second monomer comprises a VL1-CL1 monomer, wherein VL1 is a first variable light domain. The second monomer includes from N-terminus to C-terminus, a first variable light chain and a first constant CL domain. In some further embodiments, the first variable heavy domain and the first variable light domain form a first antigen binding domain that binds to a first target antigen of interest. In some embodiments, the third monomer comprises a VH2-CH1-hinge-CH2-CH3 monomer, wherein VH2 is a second variable heavy domain and CH2-CH3 is a second variant IgG Fc domain. For instance, the third monomer includes from N-terminus to C-terminus, a second variable heavy domain, a second CH1 domain, a second hinge domain, a second CH2 domain, and a second CH3 domain. In some embodiments, the fourth monomer comprises a VL2-CL2 monomer, wherein VL2 is a second variable light domain. For instance, the fourth monomer includes a second variable light domain and a second constant CL domain. In some further embodiments, the second variable heavy domain and the second variable light domain form a second antigen binding domain that binds to a second target antigen of interest (or, in some other embodiments, to the first target antigen of interest). In some embodiments, the first and second antigen binding domains of the multimeric protein bind to different antigens.

In some embodiments, the multimeric protein comprises one set of CH1:CL interface variants. In some embodiments, either (1) the first monomer and the second monomer comprise a set of CH1:CL interface variants, or (2) the third monomer and the fourth monomer comprise a set of CH1:CL interface variants, as described in further detail below. In other words, in some instances the first monomer and the second monomer comprise the set of CH1:CL variants and the third monomer and the fourth monomer do not comprise the set of CH1:CL variants, whereas in other instances the third monomer and the fourth monomer comprise the set of CH1:CL variants and the first monomer and the second monomer do not comprise the set of CH1:CL variants. In some further embodiments, the set of CH1:CL interface variants comprises a set of CH1:CL electrostatic variants, as described in further detail below. In other embodiments, the set of CH1:CL interface variants comprises a set of CH1:CL steric variants, as described in further detail below.

In some embodiments, the multimeric protein comprises a set of CH1:CL electrostatic variants and a set of CH1:CL steric variants. In some embodiments, the first monomer and the second monomer comprise the set of CH1:CL electrostatic variants, and the third monomer and the fourth monomer comprise the set of CH1:CL steric variants. Alternatively, the first monomer and the second monomer may comprise the set of CH1:CL steric variants, and the third monomer and the fourth monomer may comprise the set of CH1:CL electrostatic variants. In some embodiments, the first monomer and the second monomer comprise the set of CH1:CL electrostatic variants and the set of CH1:CL steric variants, and the third monomer and the fourth monomer do not comprise either set. In other embodiments, the third monomer and the fourth monomer comprise the set of CH1:C1 electrostatic variants and the set of CH1:CL steric variants, and the first monomer and the second monomer do not comprise either set.

In some embodiments, the multimeric protein comprises one set of VH:VL interface electrostatic variants, as described in further detail below. In some embodiments, either (1) the first monomer and the second monomer comprise a set of VH:VL electrostatic variants, or (2) the third monomer and the fourth monomer comprise a set of VH:VL electrostatic variants. In other words, in some instances, the first monomer and the second monomer comprise the set of VH:VL electrostatic variants and the third monomer and the fourth monomer do not comprise the set of VH:VL electrostatic variants, whereas in other instances the third monomer and the fourth monomer comprise the set of VH:VL electrostatic variants and the first monomer and the second monomer do not comprise the set of VH:VL electrostatic variants.

In some embodiments, the multimeric protein comprises a first set of VH:VL electrostatic variants and a second set of VH:VL electrostatic variants. In some embodiments, the first monomer and the second monomer comprise the first set of VH:VL electrostatic variants, and the third monomer and the fourth monomer comprise the second set of VH:VL electrostatic variants. In some embodiments, the first and second set of VH:VL electrostatic variants include the same variants, such as, but not limited to, the same amino acid substitutions. For instance, the amino acid substitutions of the first set of VH:VL electrostatic variants are identical to the amino acid substitutions of the first second of VH:VL electrostatic variants.

In some embodiments, the multimeric protein comprises a set of CH1:CL interface variants and a set of VH:VL interface electrostatic variants. In some embodiments, the first monomer and the second monomer comprise the set of CH1:CL interface variants, and the third monomer and the fourth monomer comprise the set of VH:VL electrostatic variants. In some further embodiments, the set of CH1:CL interface variants comprises a set of CH1:CL electrostatic variants, whereas in other embodiments the set of CH1:CL interface variants comprises a set of CH1:CL steric variants. In some embodiments, the first monomer and the second monomer comprise the set of CH1:CL electrostatic variants, and the third monomer and the fourth monomer comprise the set of VH:VL electrostatic variants. In some embodiments, the first monomer and the second monomer comprise the set of CH1:CL steric variants, and the third monomer and the fourth monomer comprise the set of VH:VL electrostatic variants. In some embodiments, the first monomer and the second monomer comprise the set of VH:VL electrostatic variants, and the third monomer and the fourth monomer comprise the set of CH1:CL interface variants. In some further embodiments, the set of CH1:CL interface variants comprises a set of CH1:CL electrostatic variants, whereas in other embodiments the set of CH1:CL interface variants comprises a set of CH1:CL steric variants. In some embodiments, the first monomer and the second monomer comprise the set of VH:VL electrostatic variants, and the third monomer and the fourth monomer comprise the set of CH1:CL electrostatic variants. In some embodiments, the first monomer and the second monomer comprise the set of VH:VL electrostatic variants, and the third monomer and the fourth monomer comprise the set of CH1:CL steric variants. In some embodiments, the first monomer and second monomer comprise the set of CH1:CL interface variants and the set of VH:VL interface variants (and the third monomer and the fourth monomer do not comprise either set of interface variants). In some further embodiments, the set of CH1:CL interface variants comprises a set of CH1:CL electrostatic variants, whereas in other embodiments the set of CH1:CL interface variants comprises a set of CH1:CL steric variants. In some embodiments, the first monomer and second monomer comprise the set of CH1:CL electrostatic variants and the set of VH:VL interface variants (and the third monomer and the fourth monomer do not comprise either set of interface variants). In some embodiments, the first monomer and second monomer comprise the set of CH1:CL steric variants and the set of VH:VL interface variants (and the third monomer and the fourth monomer do not comprise either set of interface variants). In some embodiments, the third monomer and fourth monomer comprise the set of CH1:CL interface variants and the set of VH:VL interface variants (and the first monomer and the second monomer do not comprise either set of interface variants). In some further embodiments, the set of CH1:CL interface variants comprises a set of CH1:CL electrostatic variants, whereas in other embodiments the set of CH1:CL interface variants comprises a set of CH1:CL steric variants. In some embodiments, the third monomer and fourth monomer comprise the set of CH1:CL electrostatic variants and the set of VH:VL interface variants (and the first monomer and the second monomer do not comprise either set of interface variants). In some embodiments, the third monomer and fourth monomer comprise the set of CH1:CL steric variants and the set of VH:VL interface variants (and the first monomer and the second monomer do not comprise either set of interface variants).

In some embodiments, the multimeric protein comprises a set of CH1:CL electrostatic variants, a set of CH1:CL steric variants, and a set of VH:VL interface electrostatic variants. In some embodiments, the first monomer and the second monomer comprise the set of CH1:CL electrostatic variants, the set of CH1:CL steric variants, and the set of VH:VL electrostatic variants (and the third monomer and the fourth monomer do not comprise any of the sets of interface variants). In other embodiments, the third monomer and the fourth monomer comprise the set of CH1:CL electrostatic variants, the set of CH1:CL steric variants, and the set of VH:VL electrostatic variants (and the first monomer and the second monomer do not comprise any of the sets of interface variants). In some embodiments, the first monomer and the second monomer comprise the set of CH1:CL electrostatic variants and the set of CH1:CL steric variants, and the third monomer and the fourth monomer comprise the set of VH:VL electrostatic variants. In other embodiments, the first monomer and the second monomer comprise the set of VH:VL electrostatic variants, and the third monomer and the fourth monomer comprise the set of CH1:CL electrostatic variants and the set of CH1:CL steric variants. In some embodiments, the first monomer and the second monomer comprise the set of CH1:CL electrostatic variants and the set of VH:VL electrostatic variants, and the third monomer and the fourth monomer comprise the set of CH1:CL steric variants. In other embodiments, the first monomer and the second monomer comprise the set of CH1:CL steric variants, and the third monomer and the fourth monomer comprise the set of CH1:CL electrostatic variants and the set of VH:VL electrostatic variants. In some embodiments, the first monomer and the second monomer comprise the set of CH1:CL steric variants and the set of VH:VL electrostatic variants, and the third monomer and the fourth monomer comprise the set of CH1:CL electrostatic variants. In other embodiments, the first monomer and the second monomer comprise the set of CH1:CL electrostatic variants, and the third monomer and the fourth monomer comprise the set of CH1:CL steric variants and the set of VH:VL electrostatic variants.

In some embodiments, the multimeric protein comprises a set of CH1:CL interface variants, a first set of VH:VL electrostatic variants, and a second set of VH:VL electrostatic variants. In some embodiments, the first monomer and the second monomer comprise the set of CH1:CL interface variants and the first set of VH:VL electrostatic variants, and the third monomer and the fourth monomer comprise the second set of VH:VL electrostatic variants. In some further embodiments, the set of CH1:CL interface variants comprises a set of CH1:CL electrostatic variants, whereas in other embodiments the set of CH1:CL interface variants comprises a set of CH1:CL steric variants. In some embodiments, the first monomer and the second monomer comprise the first set of VH:VL electrostatic variants, and the third monomer and the fourth monomer comprise the set of CH1:CL interface variants and the second set of VH:VL electrostatic variants. In some further embodiments, the set of CH1:CL interface variants comprises a set of CH1:CL electrostatic variants, whereas in other embodiments the set of CH1:CL interface variants comprises a set of CH1:CL steric variants.

In some embodiments, the multimeric protein comprises a set of CH1:CL electrostatic variants, a set of CH1:CL steric variants, a first set of VH:VL electrostatic variants, and a second set of VH:VL electrostatic variants. In some embodiments, the first monomer and the second monomer comprise the set of CH1:CL electrostatic variants, the set of CH1:CL steric variants, and the first set of VH:VL electrostatic variants, and the third monomer and the fourth monomer comprise the second set of VH:VL electrostatic variants. In other embodiments, the first monomer and the second monomer comprise the first set of VH:VL electrostatic variants, and the third monomer and the fourth monomer comprise the set of CH1:CL electrostatic variants, the set of CH1:CL steric variants, and the second set of VH:VL electrostatic variants. In some embodiments, the first monomer and the second monomer comprise the set of CH1:CL electrostatic variants and the first set of VH:VL electrostatic variants, and the third monomer and the fourth monomer comprise the set of CH1:CL steric variants and the second set of VH:VL electrostatic variants. In other embodiments, the first monomer and the second monomer comprise the set of CH1:CL steric variants and the first set of VH:VL electrostatic variants, and the third monomer and the fourth monomer comprise the set of CH1:CL electrostatic variants and the second set of VH:VL electrostatic variants.

As will be appreciated by those in the art, all of the recited interface variants (e.g., CH1:CL electrostatic variants, CH1:CL steric variants, VH:VL electrostatic variants, etc.) can be optionally and independently combined in any way, as long as they retain their “strandedness” or “monomer partition.” In addition, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

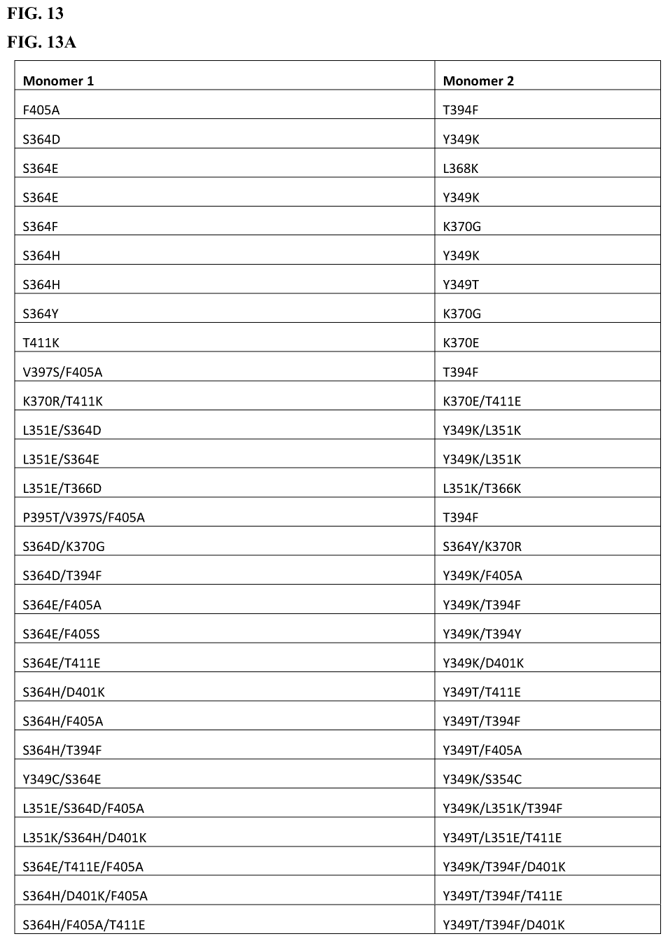

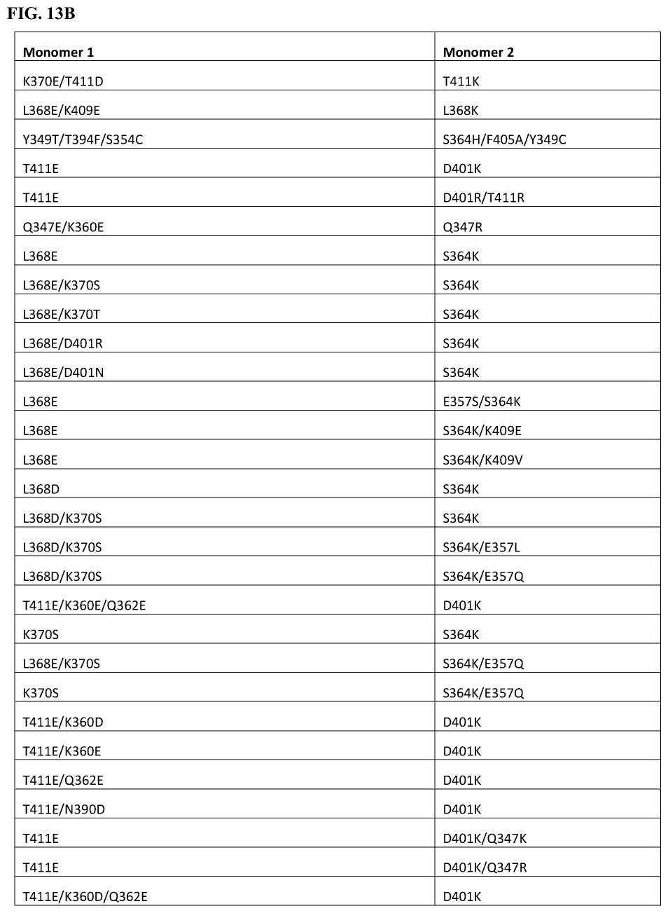

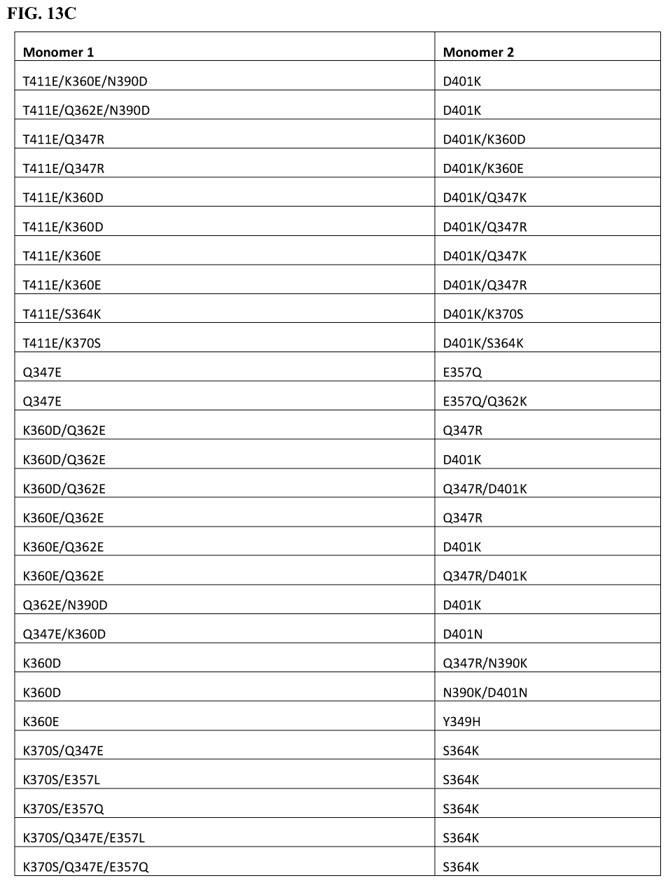

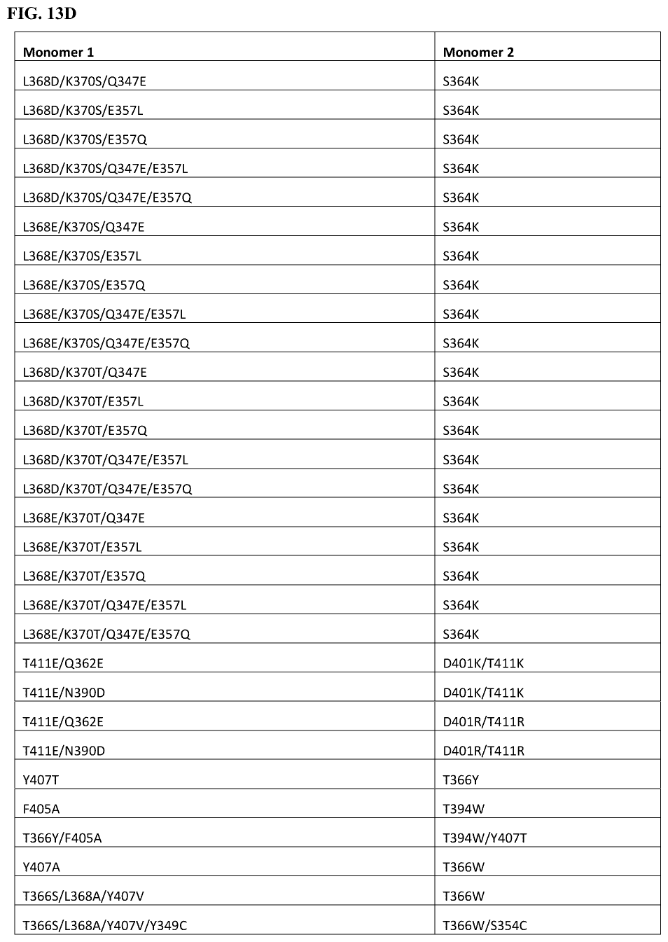

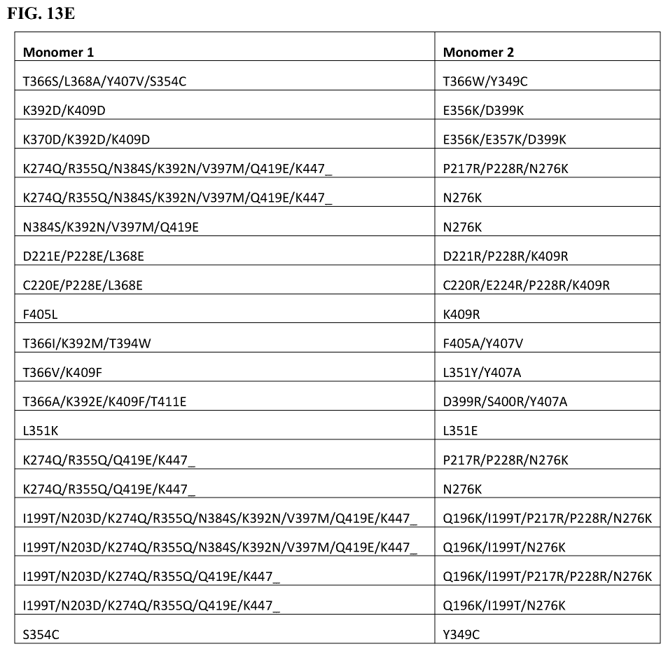

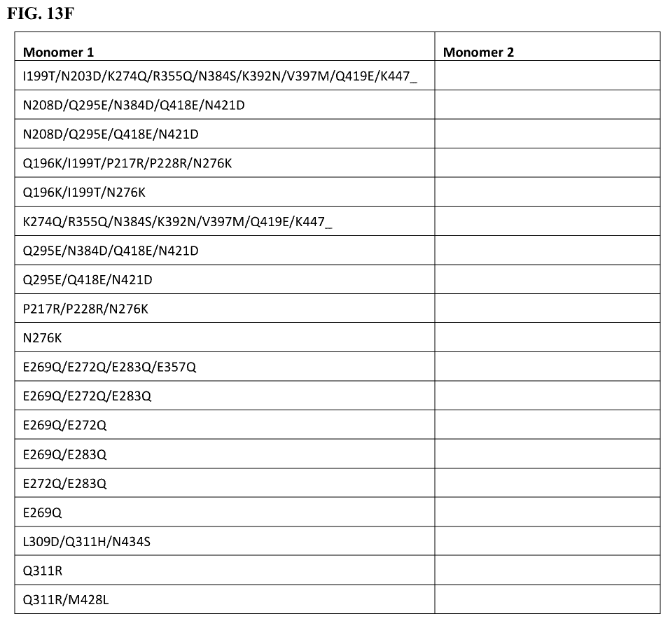

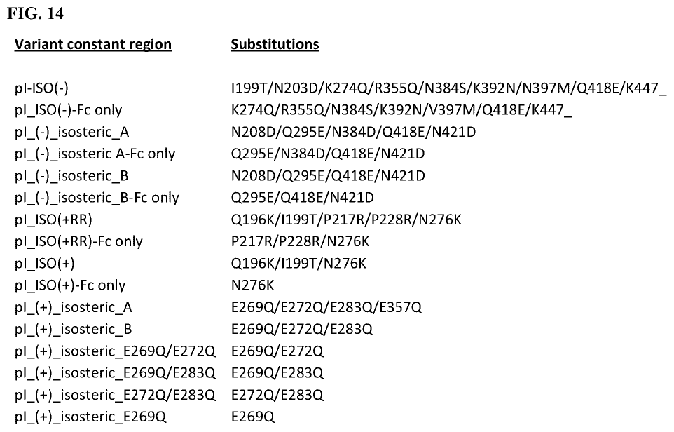

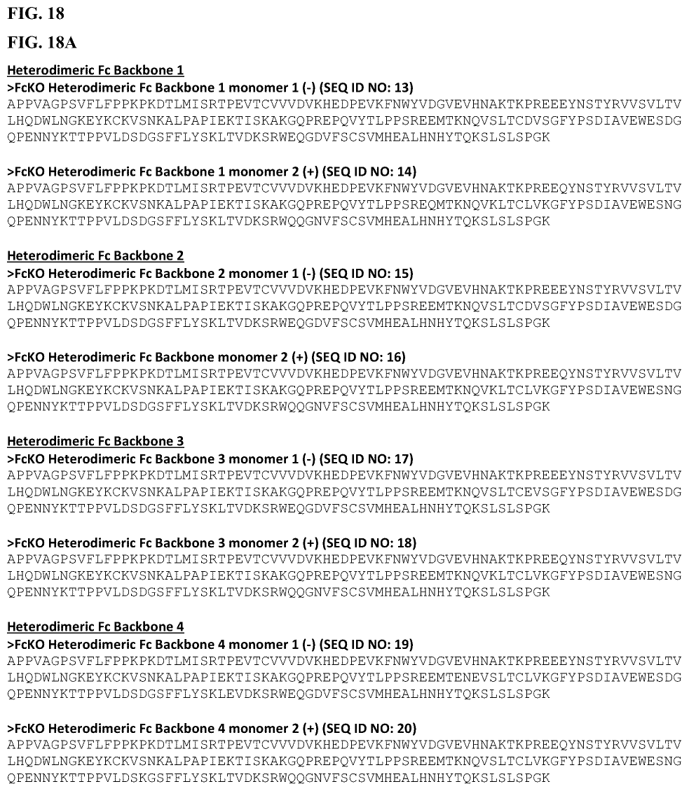

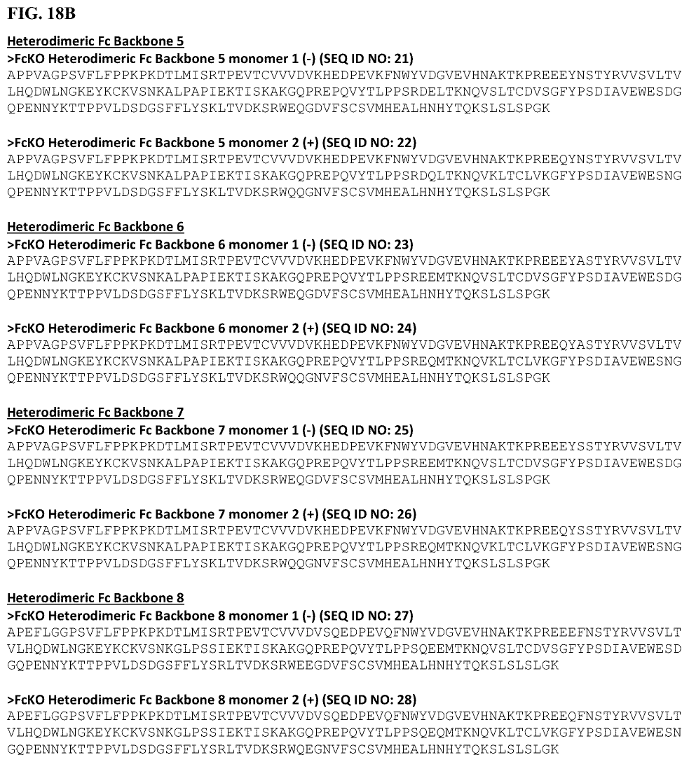

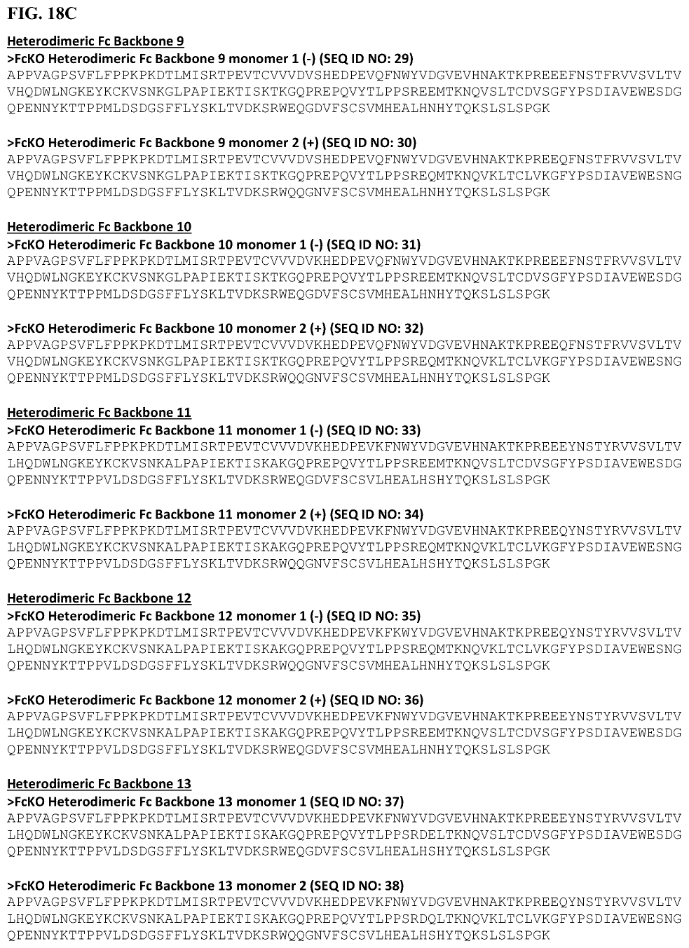

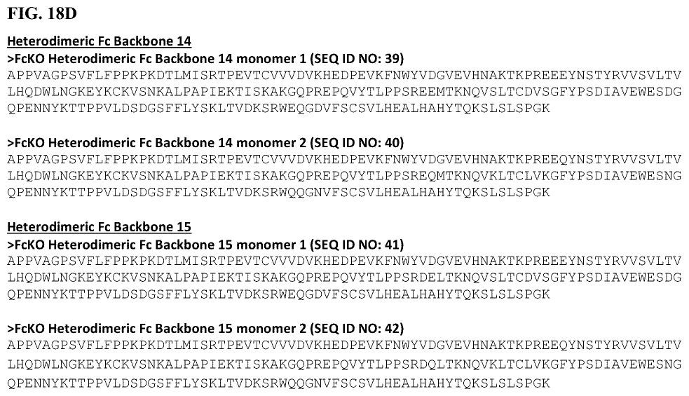

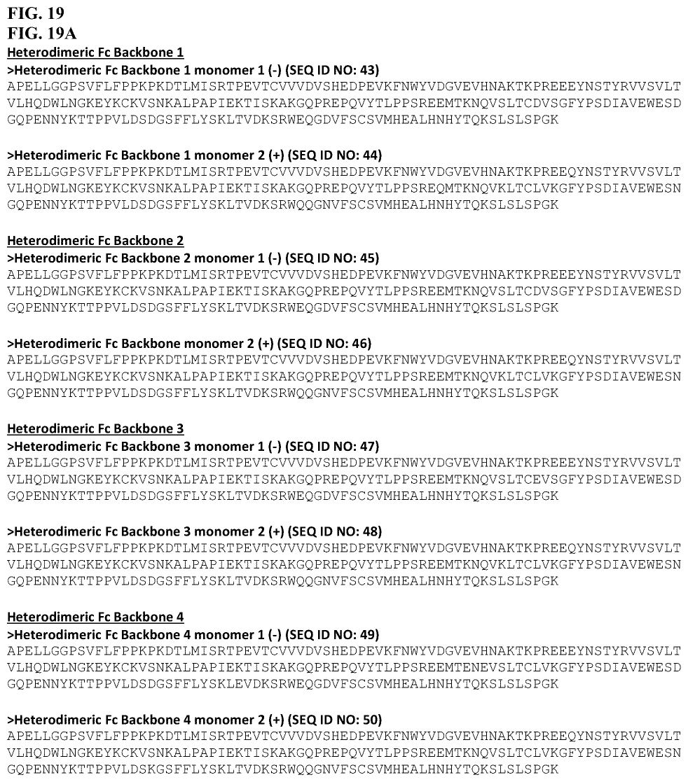

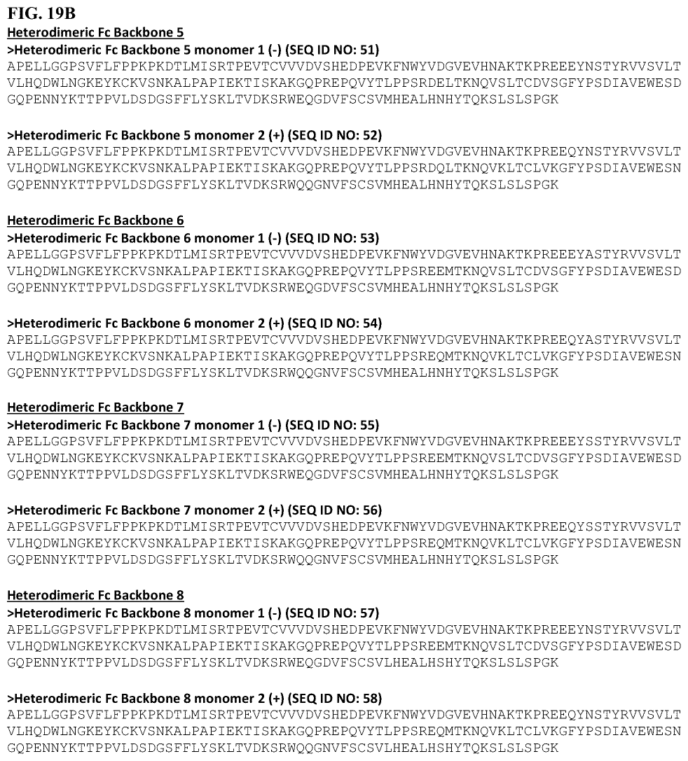

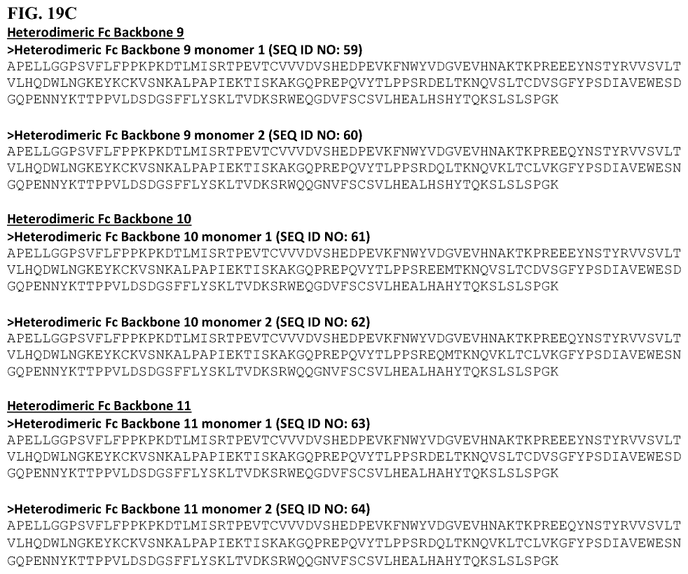

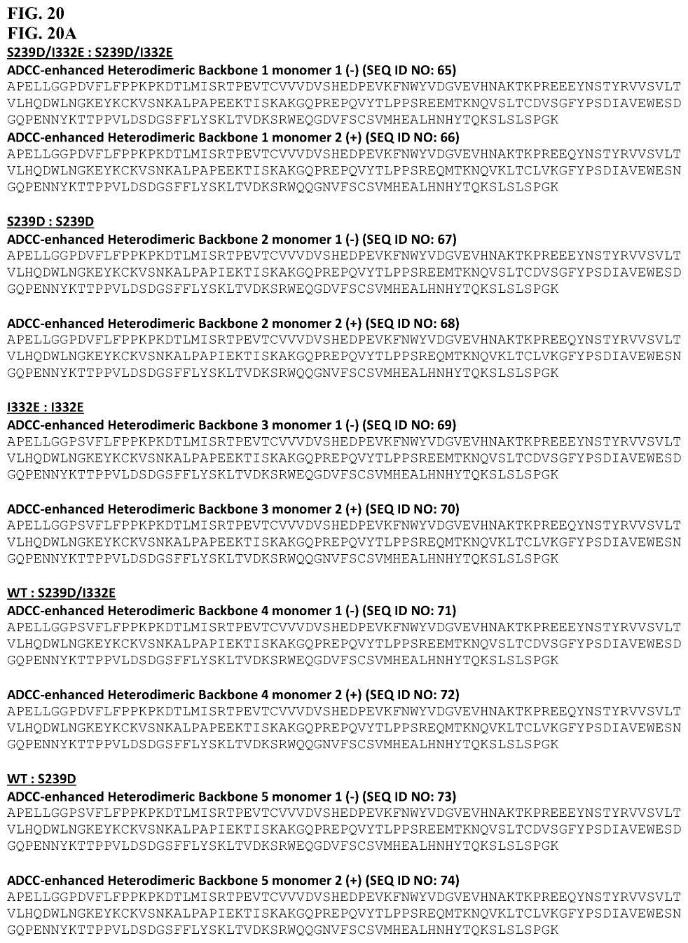

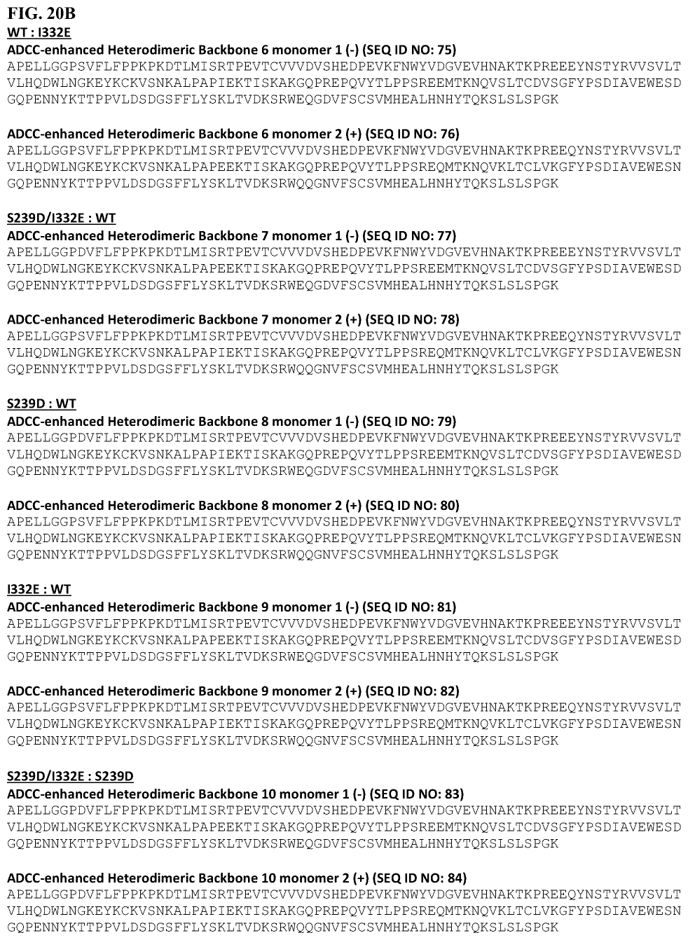

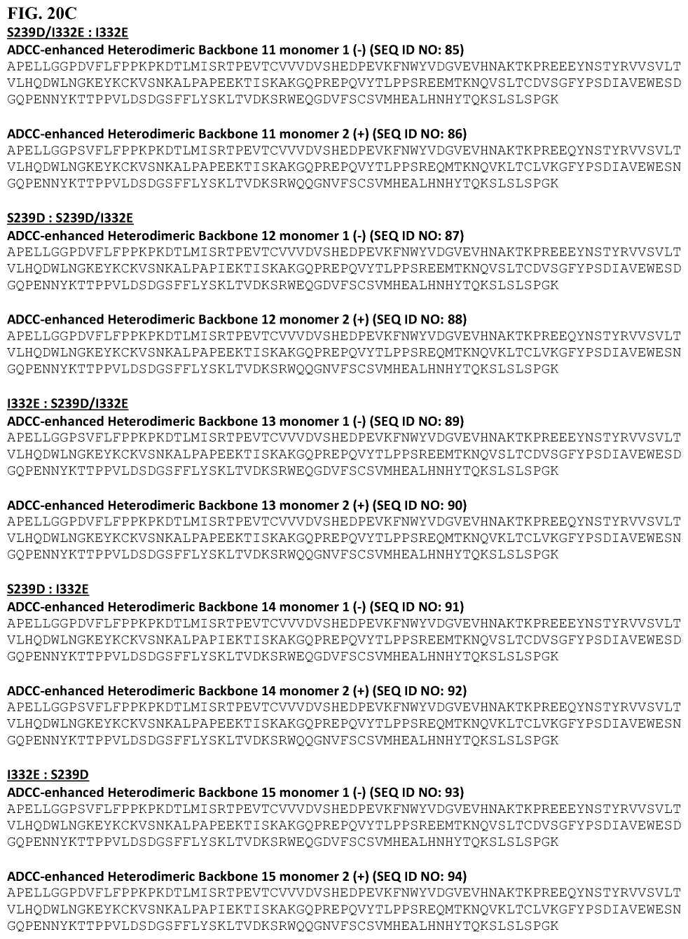

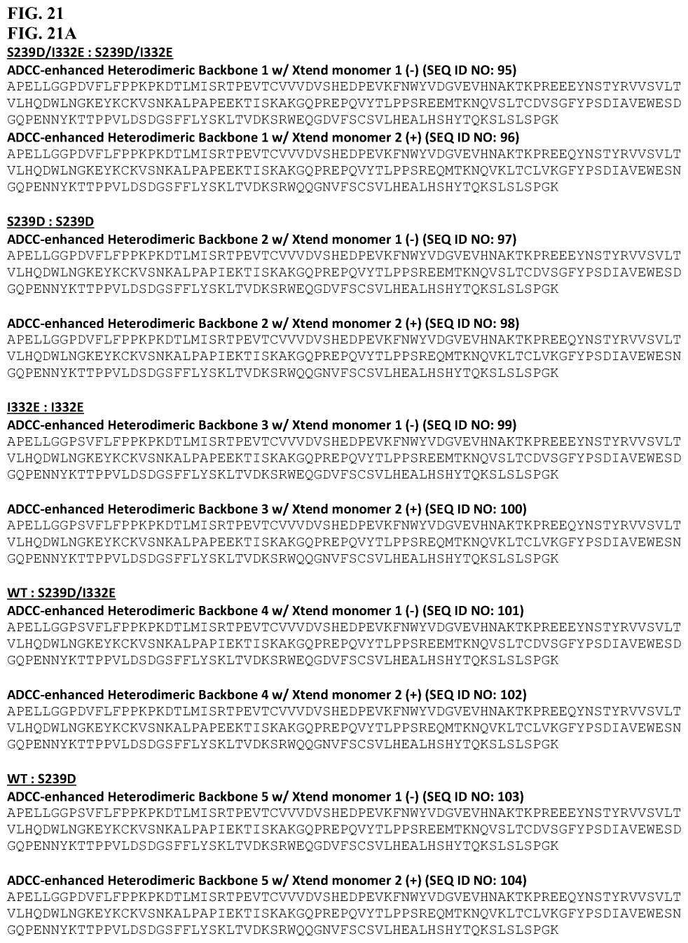

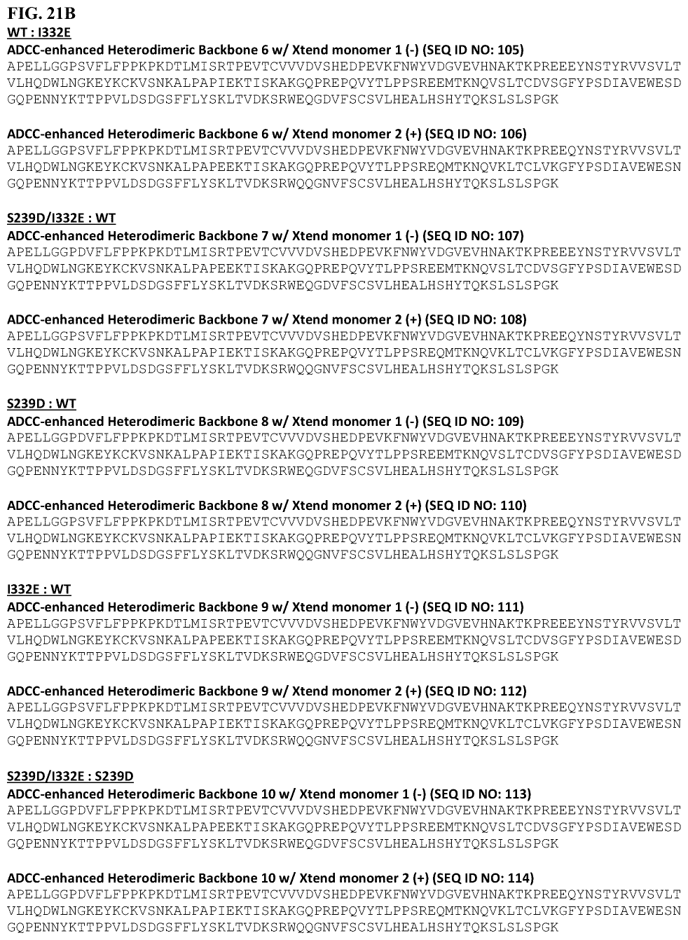

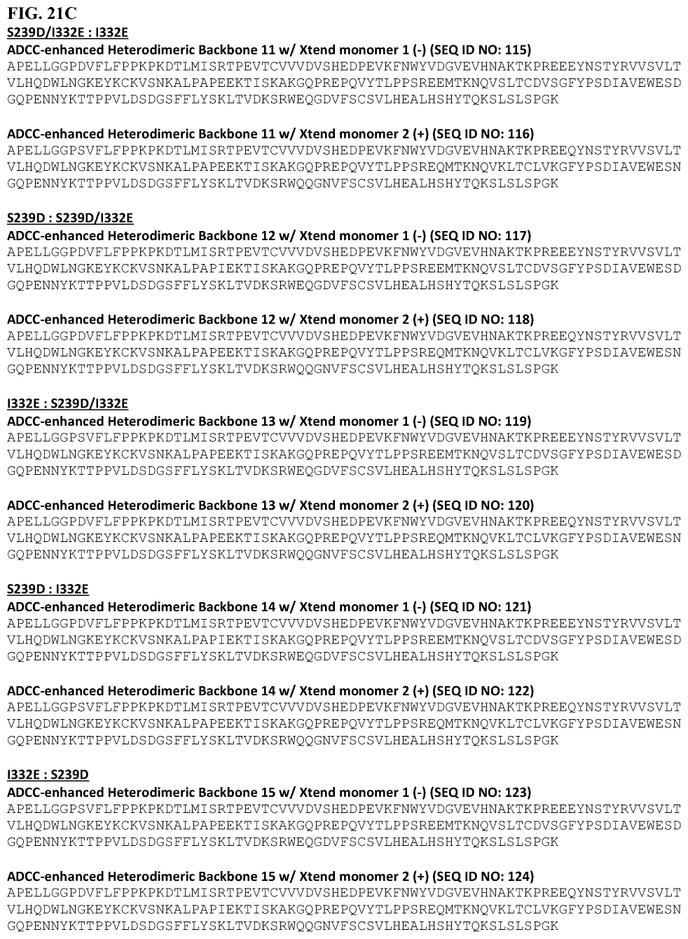

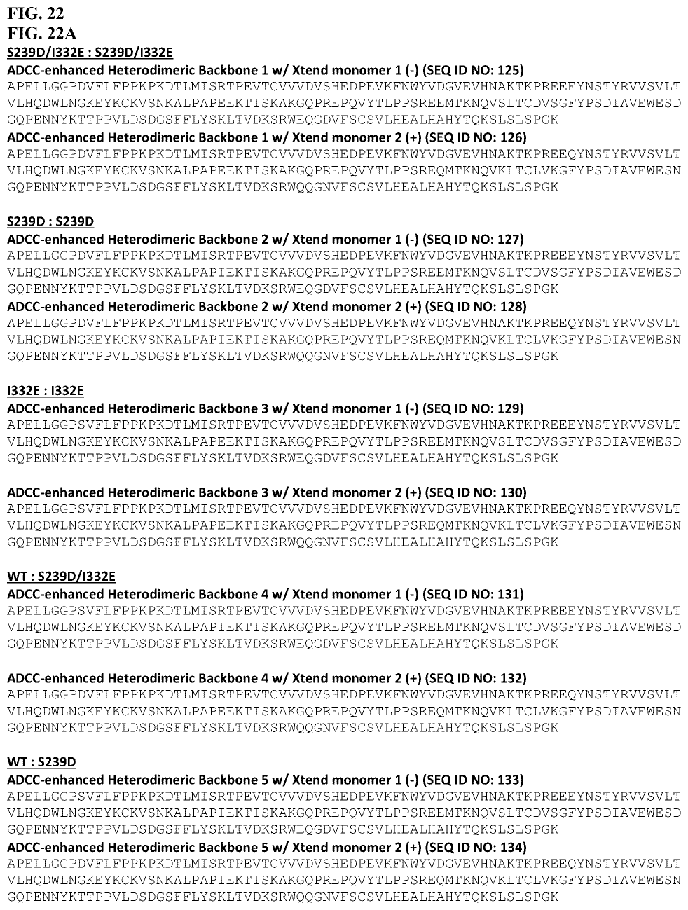

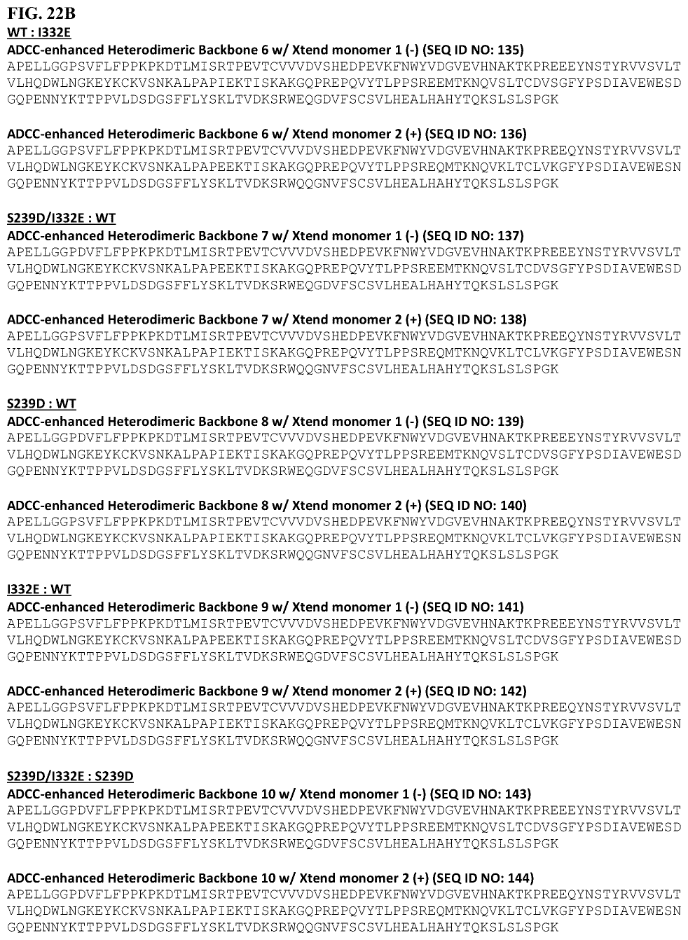

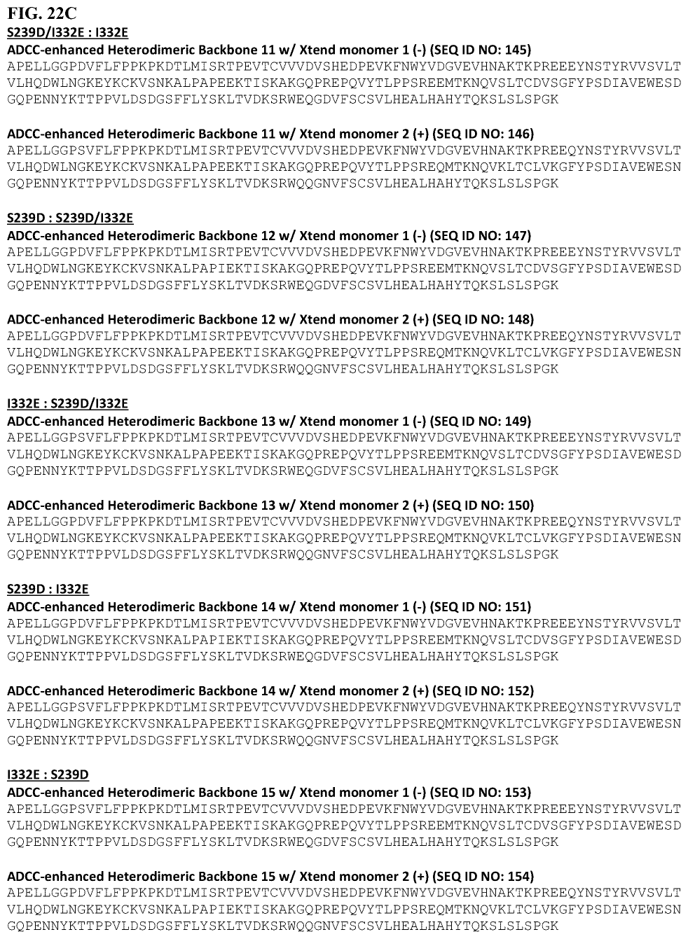

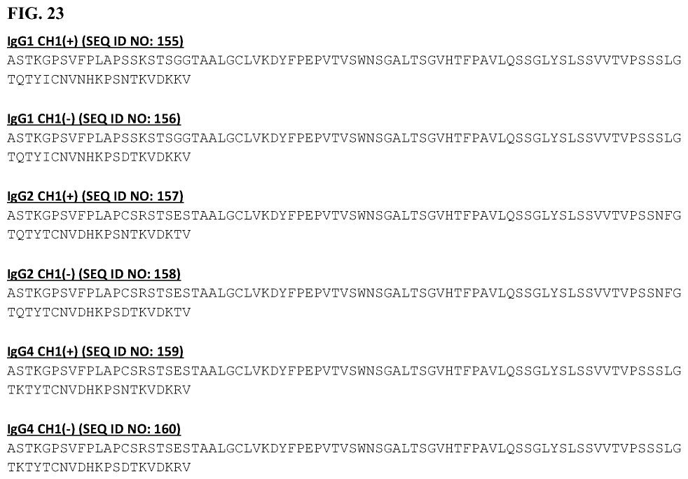

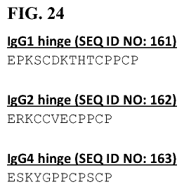

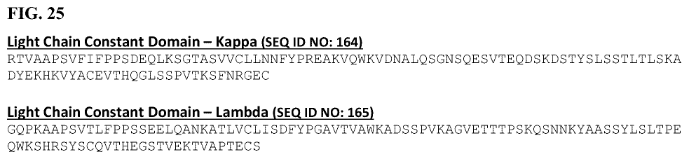

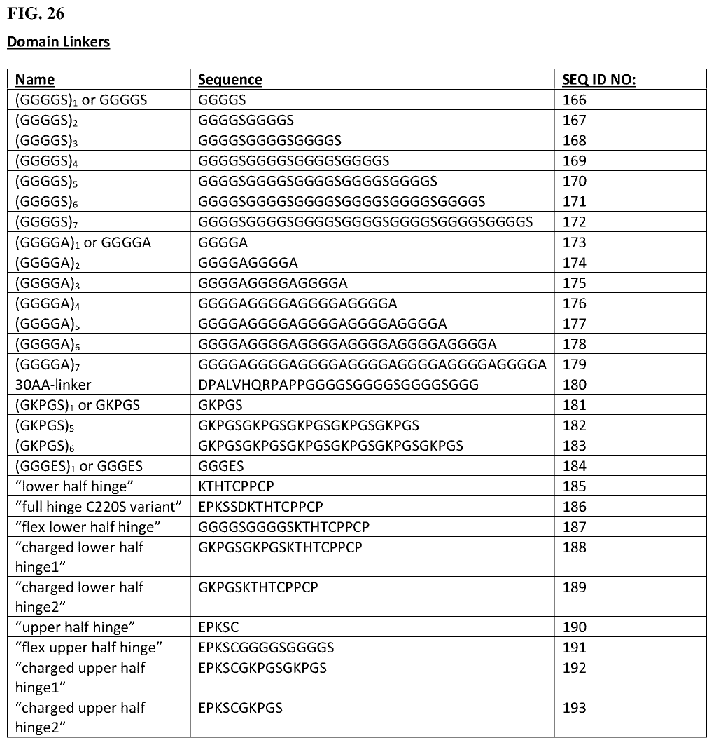

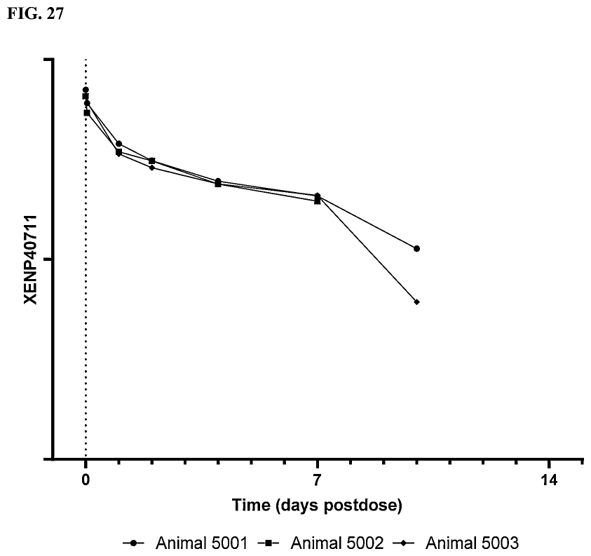

In another aspect, the subject multimeric protein comprises: (a) a variant human IgG1 CH1 domain (vCH1); and (b) a variant human kappa CL domain (vCL), wherein the CH1 domain and the CL domain together have a first set of amino acid substitutions. In some further embodiments, the first set of amino acid substitutions is selected from the group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering. For the sake of clarity, while residue K218 can be considered to be a part of the hinge region according to EU numbering, it is expressly contemplated that, when discussed in the context of a CH1:CL interface variant (such as, for example, a CH1:CL electrostatic variant) or a set of CH1:CL interface variants (such as, for example, a set of CH1:CL electrostatic variants), residue K218 is considered to be a part of the CH1 domain, even in instances where amino acid substitutions of this residue are characterized as “according to EU numbering.” In some other further embodiments, the first set of amino acid substitutions is selected from the group including: (i) A141F:F118A, (ii) A141F:F116A, and (iii) K147S:S131K, wherein numbering is according to EU numbering. As will be appreciated by those in the art, any of the interface variants can also independently and optionally be combined with one or more of the following Fc domain variants: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below. Any of the multimeric proteins including the tetrameric proteins can also have one or more amino acid substitutions (or one or more sets of amino acid substitutions) described herein in the Fc domains set forth in the Figures including but not limited to, FIGS. 13A-13F, 14, 15, 16, and 17 or in the heterodimeric Fc monomers set forth in the Figures including but not limited to FIGS. 18A-18D, 19A-19C, 20A-20C, 21A-21C, and 22A-22C.

In another aspect, the subject multimeric protein comprises: (a) a variant human IgG1 CH1 domain (vCH1); and (b) a variant human kappa CL domain (vCL), wherein: (1) the CH1 domain and the CL domain together have a first set of amino acid substitutions selected from the group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R; (2) the CH1 domain and the CL domain together have a second set of amino acid substitutions selected from the group including: (i) A141F:F118A, (ii) A141F:F116A, and (iii) K147S:S131K; and (3) numbering of the first set of amino acid substitutions and numbering of the second set of amino acid substitutions are both according to EU numbering. For the sake of clarity, while residue K218 can be considered to be a part of the hinge region according to EU numbering, it is expressly contemplated that, when discussed in the context of a CH1:CL interface variant (such as, for example, a CH1:CL electrostatic variant) or a set of CH1:CL interface variants (such as, for example, a set of CH1:CL electrostatic variants), residue K218 is considered to be a part of the CH1 domain, even in instances where amino acid substitutions of this residue are characterized as “according to EU numbering.” Any of the multimeric proteins comprising one or more sets of interface variants (e.g., CH1:CL electrostatic variant, CH1:CL steric variant, VH:VL electrostatic variant) can also have one or more amino acid substitutions (or one or more sets of amino acid substitutions) in a CH2 domain and/or CH3 domain of an IgG Fc domain (e.g., a first (variant) Fc domain, a second (variant) Fc domain), including but not limited to: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below. Any of the multimeric proteins can also have one or more amino acid substitutions (or one or more sets of amino acid substitutions) described herein in the IgG Fc domains set forth in the Figures including but not limited to, FIGS. 13A-13F, 14, 15, 16, and 17 or in the heterodimeric Fc monomers set forth in the Figures including but not limited to FIGS. 18A-18D, 19A-19C, 20A-20C, 21A-21C, and 22A-22C.

### B. Dimeric Proteins

In another aspect, the subject multimeric protein is a dimeric protein (i.e., a multimeric protein comprising two monomers). In such instances, the term “monomer” is meant one half of the multimeric protein. In embodiments where the multimeric protein is a dimeric protein, the subject multimeric protein generally comprises: (a) a first monomer, and (b) a second monomer.

In some embodiments, the first monomer comprises a VH-CH1 domain, wherein VH is a variable heavy domain. In some embodiments, the second monomer comprises a VL-CL domain, wherein VL is a variable light domain. In some further embodiments, the variable heavy domain and the variable light domain form an antigen binding domain, wherein the antigen binding domain binds a target antigen of interest.

In some embodiments, the dimeric protein comprises a set of CH1:CL interface variants. In some embodiments, the first monomer and the second monomer comprise the set of CH1:CL interface variants. In some further embodiments, the set of CH1:CL interface variants comprises a set of CH1:CL electrostatic variants, whereas in other embodiments the set of CH1:CL interface variants comprises a set of CH1:CL steric variants.

In some embodiments, the dimeric protein comprises a set of VH:VL interface electrostatic variants. In some embodiments, the first monomer and the second monomer comprise the set of VH:VL electrostatic variants.

In some embodiments, the dimeric protein comprises a set of CH1:CL interface variants and a set of VH:VL interface electrostatic variants. In some embodiments, the first monomer and the second monomer comprise the set of CH1:CL interface variants and the set of VH:VL electrostatic variants. In some further embodiments, the set of CH1:CL interface variants comprises a set of CH1:CL electrostatic variants, whereas in other embodiments the set of CH1:CL interface variants comprises a set of CH1:CL steric variants.

In some embodiments, the dimeric protein comprises a set of CH1:CL electrostatic variants and a set of CH1:CL steric variants. In some embodiments, the first monomer and the second monomer comprise the set of CH1:CL electrostatic variants and the set of CH1:CL steric variants.

In some embodiments, the dimeric protein comprises a set of CH1:CL electrostatic variants, a set of CH1:CL steric variants, and a set of VH:VL electrostatic variants. In some embodiments, the first monomer and the second monomer comprise the set of CH1:CL electrostatic variants, the set of CH1:CL steric variants, and the set of VH:VL electrostatic variants.

In some embodiments, the dimeric protein may be attached to some other protein. In some further embodiments, the multimeric protein is attached to another protein through the use of a covalent bond, whereas in some other embodiments the multimeric protein is attached to another protein through the use of a noncovalent bond.

As will be appreciated by those in the art, all of the recited interface variants (e.g., CH1:CL electrostatic variants, CH1:CL steric variants, VH:VL electrostatic variants, etc.) can be optionally and independently combined in any way, as long as they retain their “strandedness” or “monomer partition.” Further, any of the multimeric proteins can also independently and optionally be produced in a cell line that eliminates or reduces the incorporation of fucose into the glycosylation of the multimeric protein, as described in further detail below.

In another aspect, the subject dimeric protein comprises: (a) a variant human IgG1 CH1 domain (vCH1); and (b) a variant human kappa CL domain (vCL), wherein the CH1 domain and the CL domain together have a first set of amino acid substitutions. In some further embodiments, the first set of amino acid substitutions is selected from the group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering. In some other further embodiments, the first set of amino acid substitutions is selected from the group including: (i) A141F:F118A, (ii) A141F:F116A, and (iii) K147S:S131K, wherein numbering is according to EU numbering. For the sake of clarity, while residue K218 can be considered to be a part of the hinge region according to EU numbering, it is expressly contemplated that, when discussed in the context of a CH1:CL interface variant (such as, for example, a CH1:CL electrostatic variant) or a set of CH1:CL interface variants (such as, for example, a set of CH1:CL electrostatic variants), residue K218 is considered to be a part of the CH1 domain, even in instances where amino acid substitutions of this residue are characterized as “according to EU numbering.”

In another aspect, the subject dimeric protein comprises: (a) a variant human IgG1 CH1 domain (vCH1); and (b) a variant human kappa CL domain (vCL), wherein: (1) the CH1 domain and the CL domain together have a first set of amino acid substitutions selected from the group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R; (2) the CH1 domain and the CL domain together have a second set of amino acid substitutions selected from the group including: (i) A141F:F118A, (ii) A141F:F116A, and (iii) K147S:S131K; and (3) numbering of the first set of amino acid substitutions and numbering of the second set of amino acid substitutions are both according to EU numbering. For the sake of clarity, while residue K218 can be considered to be a part of the hinge region according to EU numbering, it is expressly contemplated that, when discussed in the context of a CH1:CL interface variant (such as, for example, a CH1:CL electrostatic variant) or a set of CH1:CL interface variants (such as, for example, a set of CH1:CL electrostatic variants), residue K218 is considered to be a part of the CH1 domain, even in instances where amino acid substitutions of this residue are characterized as “according to EU numbering.”

### C. Chimeric and Humanized Proteins

In certain embodiments, the multimeric proteins described herein comprise a heavy chain variable region from a particular germline heavy chain immunoglobulin gene and/or a light chain variable region from a particular germline light chain immunoglobulin gene. For example, such multimeric proteins may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally occurring somatic mutations or intentional introduction of site-directed mutation. However, a humanized multimeric protein (e.g., a humanized antibody) typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized multimeric protein may be at least 95, 96, 97, 98 or 99%, or even at least 96%, 97%, 98%, or 99% identical in amino acid sequence to the amino acid sequence encoded by the germline immunoglobulin gene. Typically, a humanized multimeric protein derived from a particular human germline sequence will display no more than 10-20 amino acid differences from the amino acid sequence encoded by the human germline immunoglobulin gene (prior to the introduction of any skew, pI, and ablation variants herein; that is, the number of variants is generally low, prior to the introduction of the variants described herein). In certain cases, the humanized multimeric protein may display no more than 5, or even no more than 4, 3, 2, or 1 amino acid difference from the amino acid sequence encoded by the germline immunoglobulin gene (again, prior to the introduction of any skew, pI, and ablation variants herein; that is, the number of variants is generally low, prior to the introduction of the variants described herein). In some embodiments, the amino acid differences are in one or more of the 6 CDRs. In some embodiments, the amino acid differences are in a VH and/or VL framework region.

In one embodiment, the parent multimeric protein (e.g., a parent antibody) has been affinity matured, as is known in the art. Structure-based methods may be employed for humanization and affinity maturation, for example as described in U.S. Pat. No. 7,657,380. Selection based methods may be employed to humanize and/or affinity mature antibody variable regions, including but not limited to methods such as string content optimization as described in U.S. Pat. No. 7,657,380 as well as method described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910⁻⁸⁹¹⁵; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### IV. Interface Variants

As described in further detail above, the present invention is directed to the generation of multimeric proteins. In some embodiments, the subject multimeric protein comprises one, two, three, four or more interface variants, such as variants at the interface of a variable heavy chain domain and a variable light chain domain (VH:VL) and variants at the interface of a constant heavy chain domain and a constant light chain domain (CH1:CL). In some embodiments, the subject multimeric protein comprises a first pair of monomers (e.g., a first monomer and a second monomer) and a second pair of monomers (e.g., a third monomer and a fourth monomer) (e.g., in instances where the multimeric protein is provided as a tetrameric protein), whereas in other embodiments the subject multimeric protein may only comprise a first pair of monomers (i.e., a first monomer and a second monomer; e.g., in instances where the multimeric protein is provided as a dimeric protein).

In instances where the subject multimeric protein is provided as a tetrameric protein, a first pair of monomers (such as, for example, the first monomer and the second monomer, or the third monomer and the fourth monomer) may comprise one, two, three or more interface variants, and a second pair of monomers (such as, for example, the third monomer and the fourth monomer, or the first monomer and the second monomer) may not comprise interface variants. In other embodiments, the first pair of monomers (e.g., the first monomer and the second monomer, the third monomer and the fourth monomer) may comprise one, two, three or more interface variants, and the second pair of monomers (e.g., the third monomer and the fourth monomer, the first monomer and the second monomer) may comprise one, two, three or more interface variants.

In instances where the subject multimeric protein is provided as a dimeric protein, the pair of monomers (i.e., the first monomer and the second monomer) may comprise one, two, three or more interface variants.

### A. CH1:CL Interface

In some embodiments, the subject multimeric protein comprises a set of CH1:CL interface variants. In some further embodiments, the set of CH1:CL interface variants comprises a set of CH1:CL electrostatic variants. In other embodiments, the set of CH1:CL interface variants comprises a set of CH1:CL steric variants.

The term “CH1:CL interface variants” refers to a “set” or “collection” of amino acid substitutions that (1) promotes the proper pairing of multimeric protein monomers wherein at least one of the monomers includes a CH1 domain and at least one of the other monomers includes a CL domain, as well as through charge complementarity, and/or (2) dissuades, impedes, or otherwise hinders the improper pairing of multimeric protein monomers (through electrostatic charge repelling) wherein at least one of the monomers includes a CH1 domain and at least one of the other monomers includes a CL domain. Generally, the CH1:CL interface variants promote the proper pairing/dissuade the improper pairing through select mechanisms, as described below. The term “CH1:CL interface electrostatic variants” (or “CH1:CL electrostatic variants”) refers to CH1:CL interface variants that promote the proper pairing of multimeric protein monomers through the formation of a salt bridge between the CH and CL domains, as well as through charge complementarity. The term can refer to CH1:CL interface variants that dissuade the improper pairing of multimeric protein monomers through electrostatic charge repelling. The term “CH1:CL interface steric variants” (or “CH1:CL steric variants”) refers to CH1:CL variants that promote the proper pairing of multimeric protein monomers (and/or dissuade the improper pairing of multimeric protein monomers) by virtue of steric complementarity.

The CH1:CL interface variants described herein are set forth in an X:Y format, wherein X denotes one or more amino acid substitutions in the CH1 domain and Y denotes one or more amino acid substitutions in the CL domain of, for instance, a Fab. For example, the CH1:CL electrostatic variants K213/K218:D122/E123 refers to amino acid substitutions at residues K213 and K218 in the CH1 domain of a monomer and amino acid substitutions at residues D122 and E123 in the CL domain of the other monomer. As will be appreciated by those in the art, a human wildtype CH1 sequence (in other words, a human CH1 sequence without an amino acid substitution) can be any shown in FIG. 23, which are represented as SEQ ID NOS: 155-160. Further, a human wildtype CL sequence (in other words, a human CL sequence without an amino acid substitution) can be any shown in FIG. 25, which are represented as SEQ ID NO: 164 for the kappa light chain constant domain and SEQ ID NO:165 for the lambda light chain constant domain.

### 1. Electrostatic Variants:

In some embodiments, a multimeric protein is provided, wherein the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain that binds to a second target antigen of interest (or, in some other embodiments, to the first target antigen of interest), wherein: (i) either the first monomer and the second monomer or the third monomer and the fourth monomer comprise a set of CH1:CL electrostatic variants, (ii) the set of CH1:CL electrostatic variants comprises amino acid substitutions at amino acid residues K213/K218:D122/E123, and (iii) the numbering is according to EU numbering.

For the sake of clarity, while residue K218 can be considered to be a part of the hinge region according to EU numbering, it is expressly contemplated that, when discussed in the context of a CH1:CL interface variant (such as, for example, a CH1:CL electrostatic variant) or a set of CH1:CL interface variants (such as, for example, a set of CH1:CL electrostatic variants), residue K218 is considered to be a part of the CH1 domain, even in instances where amino acid substitutions of this residue are characterized as “according to EU numbering.”

In some embodiments, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering. In certain embodiments, the first and second monomers of the multimeric protein comprising any one of the CH1:CL electrostatic variants listed above as (i)-(xvi). In certain embodiments, the third and fourth monomers of the multimeric protein comprising any one of the CH1:CL electrostatic variants listed above as (i)-(xvi).

In some embodiments, the set of CH1:CL electrostatic variants comprises any one of the sets of CH1:CL electrostatic variants shown in FIG. 3. In some embodiments, the set of CH1:CL electrostatic variants comprises amino acid substitutions K213E/K218D:D122K/E123K, wherein numbering is according to EU numbering. In some embodiments, the set of CH1:CL electrostatic variants of the first and second monomers comprises amino acid substitutions K213E/K218D:D122K/E123K. In some embodiments, the set of CH1:CL electrostatic variants of the third and fourth monomers comprises amino acid substitutions K213E/K218D:D122K/E123K.

In some embodiments, the first monomer and the second monomer do not comprise the set of CH1:CL electrostatic variants. In other embodiments, the third monomer and the fourth monomer do not comprise the set of CH1:CL electrostatic variants.

In some embodiments, the multimeric protein has antigen binding affinity substantially equivalent to a corresponding multimeric protein lacking the amino acid substitutions at the CH1:CL interface. In some instances, an antigen binding domain of the multimeric protein with a set of CH1:CL electrostatic variants has an antigen binding affinity that is substantially equal to an antigen binding domain of a multimeric protein without the electrostatic variants.

As will be appreciated by those in the art, the CH1:CL electrostatic variants can be optionally and independently combined in any way with any of the other types of interface variants (e.g., CH1:CL steric variants, VH:VL electrostatic variants, etc.), as long as they retain their “strandedness” or “monomer partition.” In addition, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

In some embodiments, a multimeric protein is provided, wherein the multimeric protein comprises: (a) a first monomer comprising a VH-CH1 domain, wherein VH is a variable heavy domain; and (b) a second monomer comprising a VL-CL domain, wherein VL is a variable light domain, wherein: (i) the variable heavy domain and the variable light domain form an antigen binding domain that binds to a target antigen of interest, and (ii) the first monomer and the second monomer comprise a set of CH1:CL electrostatic variants comprising amino acid substitutions at amino acid residues K213/K218:D122/E123, wherein numbering is according to EU numbering.

In some embodiments, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering.

In some embodiments, the set of CH1:CL electrostatic variants comprises amino acid substitutions K213E/K218D:D122K/E123K, wherein numbering is according to EU numbering.

In some embodiments, the set of CH1:CL electrostatic variants comprises any one of the sets of CH1:CL electrostatic variants shown in FIG. 3. In some embodiments, the set of CH1:CL electrostatic variants comprises amino acid substitutions K213E/K218D:D122K/E123K, wherein numbering is according to EU numbering. In some embodiments, the set of CH1:CL electrostatic variants of the first and second monomers comprises amino acid substitutions K213E/K218D:D122K/E123K.

In some embodiments, the multimeric protein has antigen binding affinity substantially equivalent to a corresponding multimeric protein lacking the amino acid substitutions at the CH1:CL interface. In some instances, an antigen binding domain of the multimeric protein with a set of CH1:CL electrostatic variants has an antigen binding affinity that is substantially equal to an antigen binding domain of a multimeric protein without the electrostatic variants.

As will be appreciated by those in the art, the CH1:CL electrostatic variants can be optionally and independently combined in any way with any of the other types of interface variants (e.g., CH1:CL steric variants, VH:VL electrostatic variants, etc.), as long as they retain their “strandedness” or “monomer partition.” In addition, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

### 2. Steric Variants:

In some embodiments, a multimeric protein is provided, wherein the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain that binds to a second target antigen of interest (or, in some other embodiments, to the first target antigen of interest), wherein: (i) either the first monomer and the second monomer or the third monomer and the fourth monomer comprise a set of CH1:CL steric variants, (ii) the set of CH1:CL steric variants comprises amino acid substitutions at amino acid residues selected from a group including: (1) A141:F116, (2) A141:F118, and (3) K147:S131, and (iii) the numbering is according to EU numbering. In some embodiments, the set of CH1:CL steric variants comprises amino acid substitutions selected from a group including: (i) A141F:F118A, (ii) A141F:F116A, and (iii) K147S:S131K, wherein numbering is according to EU numbering.

In certain embodiments, the set of CH1:CL steric variants of the first and second monomers comprises amino acid substitutions selected from a group including: (i) A141F:F118A, (ii) A141F:F116A, and (iii) K147S:S131K. In certain embodiments, the set of CH1:CL steric variants of the third and fourth monomers comprises amino acid substitutions selected from a group including: (i) A141F:F118A, (ii) A141F:F116A, and (iii) K147S:S131K.

In some embodiments, the first monomer and the second monomer do not comprise the set of CH1:CL steric variants. In other embodiments, the third monomer and the fourth monomer do not comprise the set of CH1:CL steric variants.

In some embodiments, the set of CH1:CL steric variants comprises any one of the sets of CH1:CL steric variants shown in FIG. 5.

In some embodiments, the multimeric protein has antigen binding affinity substantially equivalent to a corresponding multimeric protein lacking the amino acid substitutions at the CH1:CL interface. In some embodiments, an antigen binding domain of the multimeric protein with a set of CH1:CL steric variants has an antigen binding affinity that is substantially equal to an antigen binding domain of a multimeric protein without the steric variants.

As will be appreciated by those in the art, the CH1:CL steric variants can be optionally and independently combined in any way with any of the other types of interface variants (e.g., CH1:CL electrostatic variants, VH:VL electrostatic variants, etc.), as long as they retain their “strandedness” or “monomer partition.” In addition, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

In some embodiments, a multimeric protein is provided, wherein the multimeric protein comprises: (a) a first monomer comprising a VH-CH1 domain, wherein VH is a variable heavy domain; and (b) a second monomer comprising a VL-CL domain, wherein VL is a variable light domain, wherein: (i) the variable heavy domain and the variable light domain form an antigen binding domain that binds to a target antigen of interest, (ii) the first monomer and the second monomer comprise a set of CH1:CL steric variants comprising amino acid substitutions at amino acid residues selected from a group including: (1) A141:F116, (2) A141:F118, and (3) K147:S131, and (iii) numbering is according to EU numbering.

In some embodiments, the set of CH1:CL steric variants comprises amino acid substitutions selected from a group including: (i) A141F:F118A, (ii) A141F:F116A, and (iii) K147S:S131K, wherein numbering is according to EU numbering. In certain embodiments, the set of CH1:CL steric variants of the first and second monomers comprises amino acid substitutions selected from a group including: (i) A141F:F118A, (ii) A141F:F116A, and (iii) K147S:S131K.

In some embodiments, the set of CH1:CL steric variants comprises any one of the sets of CH1:CL steric variants shown in FIG. 5.

In some embodiments, the multimeric protein has antigen binding affinity substantially equivalent to a corresponding multimeric protein lacking the amino acid substitutions at the CH1:CL interface. In some embodiments, an antigen binding domain of the multimeric protein with a set of CH1:CL steric variants has an antigen binding affinity that is substantially equal to an antigen binding domain of a multimeric protein without the steric variants.

As will be appreciated by those in the art, the CH1:CL steric variants can be optionally and independently combined in any way with any of the other types of interface variants (e.g., CH1:CL electrostatic variants, VH:VL electrostatic variants, etc.), as long as they retain their “strandedness” or “monomer partition.” In addition, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

### B. VH:VL Interface

In some embodiments, the subject multimeric protein comprises a set of VH:VL interface variants. In some further embodiments, the set of VH:VL interface variants comprises a set of VH:VL interface electrostatic variants.

The term “VH:VL interface electrostatic variants” (or “VH:VL electrostatic variants”) refers to a “set” of amino acid substitutions that (1) promotes the proper pairing of multimeric protein monomers, and/or (2) dissuades, impedes, or otherwise hinders the improper pairing of multimeric protein monomers through the formation of a hydrogen bond pair between the VH and the VL domains of, for instance, a Fab. In some embodiments, the VH and VL domains form an antigen binding domain, wherein the antigen binding domain binds to a target antigen of interest.

Similar to the CH1:CL interface variants, the VH:VL electrostatic variants are set forth in an X:Y format, wherein X denotes one or more amino acid substitutions in the VH domain and Y denotes one or more amino acid substitutions in the VL domain. For example, the VH:VL electrostatic variants Q39:Q38 refers to an amino acid substitution at residue Q39 in the VH domain and an amino acid substitution at residue Q38 in the VL domain.

### I. Electrostatic Variants:

In some embodiments, a multimeric protein is provided, wherein the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain that binds the first target antigen of interest or a second target antigen of interest, wherein: (i) either the first monomer and the second monomer or the third monomer and the fourth monomer comprise a set of VH:VL electrostatic variants comprising amino acid substitutions at amino acid residues Q39:Q38, (ii) numbering is according to Kabat numbering, (iii) the first monomer binds to the second monomer by CH1-CL1 dimerization and VH1-VL1 association, and (iv) the third monomer binds to the fourth monomer by CH1-CL2 dimerization and VH2-VL2 association.

In some embodiments, the set of VH:VL electrostatic variants comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. In some embodiments, the set of VH:VL electrostatic variants of the first and second monomers described herein comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D. In some embodiments, the set of VH:VL electrostatic variants of the third and fourth monomers described herein comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D.

In some embodiments, the set of VH:VL electrostatic variants comprises amino acid substitutions Q39E:Q38K, wherein numbering is according to Kabat numbering.

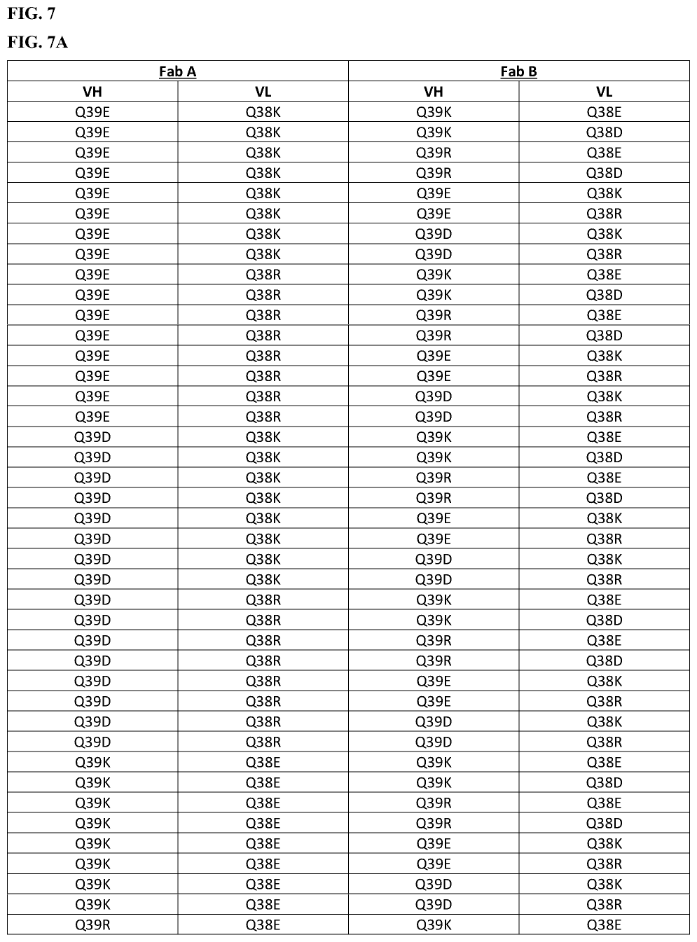

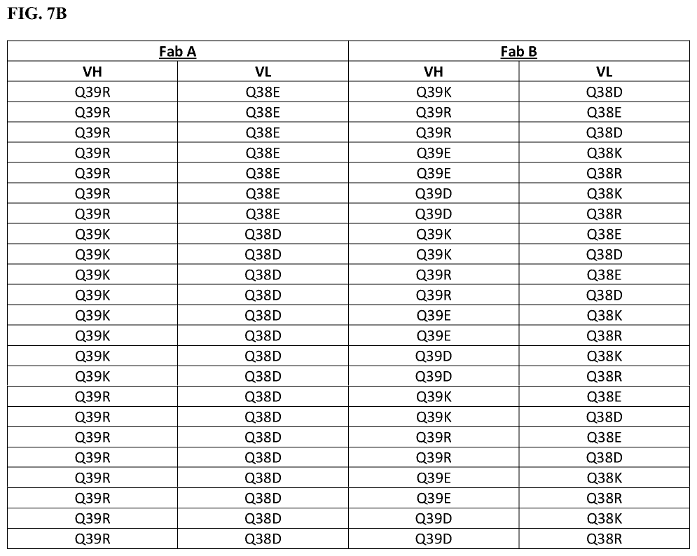

In some embodiments, the set of VH:VL electrostatic variants comprises any one of the sets of VH:VL electrostatic variants shown in FIG. 7.

In some embodiments, the multimeric protein has antigen binding affinity substantially equivalent to a corresponding multimeric protein lacking the amino acid substitutions at the VH:VL interface. In certain embodiments, an antigen binding domain of the multimeric protein with a set of VH:VL electrostatic variants has an antigen binding affinity that is substantially equal to an antigen binding domain of a multimeric protein without the electrostatic variants.

As will be appreciated by those in the art, the VH:VL electrostatic variants can be optionally and independently combined in any way with any of the other types of interface variants (e.g., CH1:CL electrostatic variants, CH1:CL steric variants, etc.), as long as they retain their “strandedness” or “monomer partition.” In addition, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

In some embodiments, a multimeric protein is provided, wherein the multimeric protein comprises: (a) a first monomer comprising a VH-CH1 domain, wherein VH is a variable heavy domain; and (b) a second monomer comprising a VL-CL domain, wherein VL is a variable light domain, wherein: (i) the variable heavy domain and the variable light domain form an antigen binding domain that binds a target antigen of interest, (ii) the first monomer and the second monomer comprise a set of VH:VL electrostatic variants comprising amino acid substitutions at amino acid residues Q39:Q38, (iii) the first monomer binds to the second monomer by CH1-CL dimerization and VH-VL association, and (iv) numbering is according to Kabat numbering.

In some embodiments, the set of VH:VL electrostatic variants comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. In some embodiments, the set of VH:VL electrostatic variants of the first and second monomers described herein comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D.

In some embodiments, the set of VH:VL electrostatic variants comprises amino acid substitutions Q39E:Q38K, wherein numbering is according to Kabat numbering.

In some embodiments, the set of VH:VL electrostatic variants comprises any one of the sets of VH:VL electrostatic variants shown in FIG. 7.

In some embodiments, the multimeric protein has antigen binding affinity substantially equivalent to a corresponding multimeric protein lacking the amino acid substitutions at the VH:VL interface. In certain embodiments, an antigen binding domain of the multimeric protein with a set of VH:VL electrostatic variants has an antigen binding affinity that is substantially equal to an antigen binding domain of a multimeric protein without the electrostatic variants.

As will be appreciated by those in the art, the VH:VL electrostatic variants can be optionally and independently combined in any way with any of the other types of interface variants (e.g., CH1:CL electrostatic variants, CH1:CL steric variants, etc.), as long as they retain their “strandedness” or “monomer partition.” In addition, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

### C. Combination of Interface Variants

As will be appreciated by those in the art, all of the recited interface variants (e.g., CH1:CL electrostatic variants, CH1:CL steric variants, VH:VL electrostatic variants, etc.) can be optionally and independently combined in any way, as long as they retain their “strandedness” or “monomer partition.” In addition, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

In some embodiments, the subject multimeric protein comprises a combination of interface variants, as set forth in further detail below. In some embodiments, the multimeric protein is provided as a tetrameric protein. In other embodiments, the multimeric protein is provided as a dimeric protein.

### 1. CH1:CL (Electrostatic)+VH:VL:

In some embodiments, a multimeric protein is provided, wherein the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein: (i) VL2 is a second variable light domain, (ii) the second variable heavy domain and the second variable light domain form a second antigen binding domain that binds to a second target antigen of interest (or, in some other embodiments, to the first target antigen of interest), (iii) the first monomer binds to the second monomer by CH1-CL1 dimerization and VH1-VL1 association, and (iv) the third monomer binds to the fourth monomer by CH1-CL2 dimerization and VH2-VL2 association.

In some embodiments, the multimeric protein further comprises a set of CH1:CL electrostatic variants and a set of VH:VL electrostatic variants.

In some embodiments, the set of CH1:CL electrostatic variants comprises amino acid substitutions at amino acid residues K213/K218:D122/E123, wherein numbering is according to EU numbering. For the sake of clarity, while residue K218 can be considered to be a part of the hinge region according to EU numbering, it is expressly contemplated that, when discussed in the context of a CH1:CL interface variant (such as, for example, a CH1:CL electrostatic variant) or a set of CH1:CL interface variants (such as, for example, a set of CH1:CL electrostatic variants), residue K218 is considered to be a part of the CH1 domain, even in instances where amino acid substitutions of this residue are characterized as “according to EU numbering.” In some embodiments, the set of CH1:CL electrostatic variants comprises amino acid substitutions K213D/E and K218D/E of the CH1 domain, and D122K/R and E123K/R of the CL domain, according to EU numbering. In some embodiments, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering. In some embodiments, the set of CH1:CL electrostatic variants comprises amino acid substitutions K213E/K218D:D122K/E123K, wherein numbering is according to EU numbering. In some embodiments, the set of CH1:CL electrostatic variants comprises any one of the sets of CH1:CL electrostatic variants shown in FIG. 3.

In some embodiments, the set of VH:VL electrostatic variants comprise amino acid substitutions at amino acid residues Q39:Q38, wherein numbering is according to Kabat numbering. In some embodiments, the set of VH:VL electrostatic variants comprise amino acid substitutions Q39D/E/K/R:Q38D/E/K/R. In some embodiments, the set of VH:VL electrostatic variants comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. In some embodiments, the set of VH:VL electrostatic variants comprises amino acid substitutions Q39E:Q38K, wherein numbering is according to Kabat numbering. In some embodiments, the set of VH:VL electrostatic variants comprises any one of the sets of VH:VL electrostatic variants shown in FIG. 7.

In some embodiments, the first monomer and the second monomer comprise the set of CH1:CL electrostatic variants and the set of VH:VL electrostatic variants (and the third monomer and the fourth monomer do not comprise the interface variants). In certain embodiments of the first and second monomers, the set of CH1:CL electrostatic variants is selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering; and the set of VH:VL electrostatic variants is selected from a group consisting of: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. Any one of the sets of CH1:CL electrostatic variants listed in (i)-(xvi) and any one of the sets of VH:VL electrostatic variants listed in (i)-(viii) can be in the first and second monomers of the multimeric protein described herein.

In some embodiments, the third monomer and the fourth monomer comprise the set of CH1:CL electrostatic variants and the set of VH:VL electrostatic variants (and the first monomer and the second monomer do not comprise the interface variants). In certain embodiments of the third and fourth monomers, the set of CH1:CL electrostatic variants is selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering; and the set of VH:VL electrostatic variants is selected from a group consisting of: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. Any one of the sets of CH1:CL electrostatic variants listed in (i)-(xvi) and any one of the sets of VH:VL electrostatic variants listed in (i)-(viii) can be found in the third and fourth monomers of the multimeric protein described herein.

In some embodiments, the first monomer and the second monomer comprise the set of CH1:CL electrostatic variants, and the third monomer and the fourth monomer comprise the set of VH:VL electrostatic variants.

In some embodiments, the first monomer and the second monomer comprise the set of VH:VL electrostatic variants, and the third monomer and the fourth monomer comprise the set of CH1:CL electrostatic variants.

In some embodiments, the multimeric protein has antigen binding affinity substantially equivalent to a corresponding multimeric protein lacking the amino acid substitutions at the VH:VL interface and the CH1:CL interface.

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

In some embodiments, a multimeric protein is provided, wherein the multimeric protein comprises: (a) a first monomer comprising a VH-CH1 domain, wherein VH is a variable heavy domain; and (b) a second monomer comprising a VL-CL domain, wherein VL is a variable light domain, and wherein the variable heavy domain and the variable light domain form an antigen binding domain that binds to a target antigen of interest, and the first monomer binds to the second monomer by CH1-CL dimerization and VH-VL association.

In some embodiments, the multimeric protein further comprises a set of CH1:CL electrostatic variants and a set of VH:VL electrostatic variants.

In some embodiments, the set of CH1:CL electrostatic variants comprises amino acid substitutions at amino acid residues K213/K218:D122/E123, wherein numbering is according to EU numbering. In some embodiments, the set of CH1:CL electrostatic variants comprises amino acid substitutions K213D/E and K218D/E of the CH1 domain, and D122K/R and E123K/R of the CL domain, according to EU numbering. In some embodiments, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering. In some embodiments, the set of CH1:CL electrostatic variants comprises amino acid substitutions K213E/K218D:D122K/E123K, wherein numbering is according to EU numbering. In some embodiments, the set of CH1:CL electrostatic variants comprises any one of the sets of CH1:CL electrostatic variants shown in FIG. 3.

In some embodiments, the set of VH:VL electrostatic variants comprise amino acid substitutions at amino acid residues Q39:Q38, wherein numbering is according to Kabat numbering. In some embodiments, the set of VH:VL electrostatic variants comprise amino acid substitutions Q39D/E/K/R:Q38D/E/K/R, according to Kabat numbering. In some embodiments, the set of VH:VL electrostatic variants comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. In some embodiments, the set of VH:VL electrostatic variants comprises amino acid substitutions Q39E:Q38K, wherein numbering is according to Kabat numbering. In some embodiments, the set of VH:VL electrostatic variants comprises any one of the sets of VH:VL electrostatic variants shown in FIG. 7.

In some embodiments of the first and second monomers, the set of CH1:CL electrostatic variants is selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering; and the set of VH:VL electrostatic variants is selected from a group consisting of: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. Any one of the sets of CH1:CL electrostatic variants listed in (i)-(xvi) and any one of the sets of VH:VL electrostatic variants listed in (i)-(viii) can be found in the first and second monomers of the multimeric protein described herein.

In some embodiments, the multimeric protein has antigen binding affinity substantially equivalent to a corresponding multimeric protein lacking the amino acid substitutions at the VH:VL interface and the CH1:CL interface.

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

### 2. CI1:CL (Steric)+VH:VL:

In some embodiments, a multimeric protein is provided, wherein the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein: (i) VL2 is a second variable light domain, (ii) the second variable heavy domain and the second variable light domain form a second antigen binding domain that binds to a second target antigen of interest (or, in some other embodiments, to the first target antigen of interest), (iii) the first monomer binds to the second monomer by CH1-CL1 dimerization and VH1-VL1 association, and (iv) the third monomer binds to the fourth monomer by CH1-CL2 dimerization and VH2-VL2 association.

In some embodiments, the multimeric protein further comprises a set of CH1:CL steric variants and a set of VH:VL electrostatic variants.

In some embodiments, the set of CH1:CL steric variants comprises amino acid substitutions at amino acid residues selected from a group including: (i) A141:F118, (ii) A141:F116, and (iii) K147:S131. In some embodiments, the set of CH1:CL steric variants comprises amino acid substitutions selected from a group including: (i) A141F:F118A, (ii) A141F:F116A, and (iii) K147S:S131K, wherein numbering is according to EU numbering. In some embodiments, the set of CH1:CL steric variants comprises any one of the sets of CH1:CL steric variants shown in FIG. 5.

In some embodiments, the set of VH:VL electrostatic variants comprise amino acid substitutions at amino acid residues Q39:Q38, wherein numbering is according to Kabat numbering. In some embodiments, the set of VH:VL electrostatic variants comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. In some embodiments, the set of VH:VL electrostatic variants comprises amino acid substitutions Q39E:Q38K, wherein numbering is according to Kabat numbering. In some embodiments, the set of VH:VL electrostatic variants comprises any one of the sets of VH:VL electrostatic variants shown in FIG. 7.

In some embodiments, the first monomer and the second monomer comprise the set of CH1:CL steric variants and the set of VH:VL electrostatic variants (and the third monomer and the fourth monomer do not comprise the interface variants). In some embodiments of the first monomer and the second monomer, the set of CH1:CL steric variants is selected from a group including: (i) A141F:F118A, (ii) A141F:F116A, and (iii) K147S:S131K, wherein numbering is according to EU numbering; and the set of VH:VL electrostatic variants is selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. Any one of the sets of CH1:CL steric variants listed in (i)-(iii) and any one of the sets of VH:VL electrostatic variants listed in (i)-(viii) can be found in the first and second monomers of the multimeric protein described herein.

In some embodiments, the third monomer and the fourth monomer comprise the set of CH1:CL steric variants and the set of VH:VL electrostatic variants (and the first monomer and the second monomer do not comprise the interface variants). In some embodiments of the third and the fourth monomer, the set of CH1:CL steric variants is selected from a group including: (i) A141F:F118A, (ii) A141F:F116A, and (iii) K147S:S131K, wherein numbering is according to EU numbering; and the set of VH:VL electrostatic variants is selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. Any one of the sets of CH1:CL steric variants listed in (i)-(iii) and any one of the sets of VH:VL electrostatic variants listed in (i)-(viii) can be found in the third and fourth monomers of the multimeric protein described herein.

In some embodiments, the first monomer and the second monomer comprise the set of CH1:CL steric variants selected from a group including: (i) A141F:F118A, (ii) A141F:F116A, and (iii) K147S:S131K, wherein numbering is according to EU numbering, and the third monomer and the fourth monomer comprise the set of VH:VL electrostatic variants selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering.

In some embodiments, the first monomer and the second monomer comprise the set of VH:VL electrostatic variants selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering, and the third monomer and the fourth monomer comprise the set of CH1:CL steric variants selected from a group including: (i) A141F:F118A, (ii) A141F:F116A, and (iii) K147S:S131K, wherein numbering is according to EU numbering.

In some embodiments, the multimeric protein has antigen binding affinity substantially equivalent to a corresponding multimeric protein lacking the amino acid substitutions at the VH:VL interface and the CH1:CL interface.

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

In some embodiments, a multimeric protein is provided, wherein the multimeric protein comprises: (a) a first monomer comprising a VH-CH1 domain, wherein VH is a variable heavy domain; and (b) a second monomer comprising a VL-CL domain, wherein VL is a variable light domain, and wherein the variable heavy domain and the variable light domain form an antigen binding domain that binds to a target antigen of interest, and the first monomer binds to the second monomer by CH1-CL dimerization and VH-VL association.

In some embodiments, the multimeric protein further comprises a set of CH1:CL steric variants and a set of VH:VL electrostatic variants.

In some embodiments, the set of CH1:CL steric variants comprises amino acid substitutions at amino acid residues selected from a group including: (i) A141:F118, (ii) A141:F116, and (iii) K147:S131. In some embodiments, the set of CH1:CL steric variants comprises amino acid substitutions selected from a group including: (i) A141F:F118A, (ii) A141F:F116A, and (iii) K147S:S131K, wherein numbering is according to EU numbering. In some embodiments, the set of CH1:CL steric variants comprises any one of the sets of CH1:CL steric variants shown in FIG. 5.

In some embodiments, the set of VH:VL electrostatic variants comprise amino acid substitutions at amino acid residues Q39:Q38, wherein numbering is according to Kabat numbering. In some embodiments, the set of VH:VL electrostatic variants comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. In some embodiments, the set of VH:VL electrostatic variants comprises amino acid substitutions Q39E:Q38K, wherein numbering is according to Kabat numbering. In some embodiments, the set of VH:VL electrostatic variants comprises any one of the sets of VH:VL electrostatic variants shown in FIG. 7.

In some embodiments of the first monomer and the second monomer, the set of CH1:CL steric variants is selected from a group including: (i) A141F:F118A, (ii) A141F:F116A, and (iii) K147S:S131K, wherein numbering is according to EU numbering; and the set of VH:VL electrostatic variants is selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. Any one of the sets of CH1:CL steric variants listed in (i)-(iii) and any one of the sets of VH:VL electrostatic variants listed in (i)-(viii) can be found in the first and second monomers of the multimeric protein described herein.

In some embodiments, the multimeric protein has antigen binding affinity substantially equivalent to a corresponding multimeric protein lacking the amino acid substitutions at the VH:VL interface and the CH1:CL interface.

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

### 3. CH1:CL (Electrostatic)+CH1:CL (Steric):

In some embodiments, a multimeric protein is provided, wherein the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein: (i) VL2 is a second variable light domain, (ii) the second variable heavy domain and the second variable light domain form a second antigen binding domain that binds to a second target antigen of interest (or, in some other embodiments, to the first target antigen of interest), (iii) the first monomer binds to the second monomer by CH1-CL1 dimerization and VH1-VL1 association, and (iv) the third monomer binds to the fourth monomer by CH1-CL2 dimerization and VH2-VL2 association.

In some embodiments, the multimeric protein further comprises a set of CH1:CL electrostatic variants and a set of CH1:CL steric variants.

In some embodiments, the set of CH1:CL electrostatic variants comprises amino acid substitutions at amino acid residues K213/K218:D122/E123, wherein numbering is according to EU numbering. For the sake of clarity, while residue K218 can be considered to be a part of the hinge region according to EU numbering, it is expressly contemplated that, when discussed in the context of a CH1:CL interface variant (such as, for example, a CH1:CL electrostatic variant) or a set of CH1:CL interface variants (such as, for example, a set of CH1:CL electrostatic variants), residue K218 is considered to be a part of the CH1 domain, even in instances where amino acid substitutions of this residue are characterized as “according to EU numbering.” In some embodiments, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering. In some embodiments, the set of CH1:CL electrostatic variants comprises amino acid substitutions K213E/K218D:D122K/E123K, wherein numbering is according to EU numbering. In some embodiments, the set of CH1:CL electrostatic variants comprises any one of the sets of CH1:CL electrostatic variants shown in FIG. 3.

In some embodiments, the set of CH1:CL steric variants comprises amino acid substitutions at amino acid residues selected from a group including: (i) A141:F118, (ii) A141:F116, and (iii) K147:S131. In some embodiments, the set of CH1:CL steric variants comprises amino acid substitutions selected from a group including: (i) A141F:F118A, (ii) A141F:F116A, and (iii) K147S:S131K, wherein numbering is according to EU numbering. In some embodiments, the set of CH1:CL steric variants comprises any one of the sets of CH1:CL steric variants shown in FIG. 5.

In some embodiments, the first monomer and the second monomer comprise the set of CH1:CL electrostatic variants and the set of CH1:CL steric variants (and the third monomer and the fourth monomer do not comprise the interface variants). In some embodiments of the first monomer and the second monomer, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering; and the set of CH1:CL steric variants is selected from a group including: (i) A141F:F118A, (ii) A141F:F116A, and (iii) K147S:S131K, wherein numbering is according to EU numbering. Any one of the sets of CH1:CL electrostatic variants listed in (i)-(xvi) and any one of the sets of CH1:CL steric variants listed in (i)-(iii) can be found in the first and second monomers of the multimeric protein described herein.

In some embodiments, the third monomer and the fourth monomer comprise the set of CH1:CL electrostatic variants and the set of CH1:CL steric variants (and the first monomer and the second monomer do not comprise the interface variants). In some embodiments of the third monomer and the fourth monomer, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering; and the set of CH1:CL steric variants is selected from a group including: (i) A141F:F118A, (ii) A141F:F116A, and (iii) K147S:S131K, wherein numbering is according to EU numbering. Any one of the sets of CH1:CL electrostatic variants listed in (i)-(xvi) and any one of the sets of CH1:CL steric variants listed in (i)-(iii) can be found in the third and fourth monomers of the multimeric protein described herein.

In some embodiments, the first monomer and the second monomer comprise the set of CH1:CL electrostatic variants selected from those listed above, and the third monomer and the fourth monomer comprise the set of CH1:CL steric variants selected from those listed above.

In some embodiments, the first monomer and the second monomer comprise the set of CH1:CL steric variants selected from those listed above, and the third monomer and the fourth monomer comprise the set of CH1:CL electrostatic variants selected from those listed above.

In some embodiments, the multimeric protein has antigen binding affinity substantially equivalent to a corresponding multimeric protein lacking the amino acid substitutions at the CH1:CL interface(s).

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

In some embodiments, a multimeric protein is provided, wherein the multimeric protein comprises: (a) a first monomer comprising a VH-CH1 domain, wherein VH is a variable heavy domain; and (b) a second monomer comprising a VL-CL domain, wherein VL is a variable light domain, and wherein the variable heavy domain and the variable light domain form an antigen binding domain that binds to a target antigen of interest, and the first monomer binds to the second monomer by CH1-CL dimerization and VH-VL association.

In some embodiments, the multimeric protein further comprises a set of CH1:CL electrostatic variants and a set of CH1:CL steric variants.

In some embodiments, the set of CH1:CL electrostatic variants comprises amino acid substitutions at amino acid residues K213/K218:D122/E123, wherein numbering is according to EU numbering. In some embodiments, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering. In some embodiments, the set of CH1:CL electrostatic variants comprises amino acid substitutions K213E/K218D:D122K/E123K, wherein numbering is according to EU numbering. In some embodiments, the set of CH1:CL electrostatic variants comprises any one of the sets of CH1:CL electrostatic variants shown in FIG. 3.

In some embodiments, the set of CH1:CL steric variants comprises amino acid substitutions at amino acid residues selected from a group including: (i) A141:F118, (ii) A141:F116, and (iii) K147:S131. In some embodiments, the set of CH1:CL steric variants comprises amino acid substitutions selected from a group including: (i) A141F:F118A, (ii) A141F:F116A, and (iii) K147S:S131K, wherein numbering is according to EU numbering. In some embodiments, the set of CH1:CL steric variants comprises any one of the sets of CH1:CL steric variants shown in FIG. 5.

In some embodiments of the first monomer and the second monomer, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering; and the set of CH1:CL steric variants is selected from a group including: (i) A141F:F118A, (ii) A141F:F116A, and (iii) K147S:S131K, wherein numbering is according to EU numbering.

In some embodiments, the multimeric protein has antigen binding affinity substantially equivalent to a corresponding multimeric protein lacking the amino acid substitutions at the CH1:CL interface(s).

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

4. VH:VL (1^{st}+VH:VL (2^{nd} Set)

In some embodiments, a multimeric protein is provided, wherein the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein: (i) VL2 is a second variable light domain, (ii) the second variable heavy domain and the second variable light domain form a second antigen binding domain that binds to a second target antigen of interest (or, in some other embodiments, to the first target antigen of interest), (iii) the first monomer binds to the second monomer by CH1-CL1 dimerization and VH1-VL1 association, and (iv) the third monomer binds to the fourth monomer by CH1-CL2 dimerization and VH2-VL2 association.

In some embodiments, the multimeric protein further comprises a first set of VH:VL electrostatic variants and a second set of VH:VL electrostatic variants.

In some embodiments, the first set of VH:VL electrostatic variants and the second set of VH:VL electrostatic variants comprise amino acid substitutions selected from a group including: (i) Q39E:Q38K and Q39K:Q38E, respectively, (ii) Q39E:Q38K and Q39K:Q38D, respectively, (iii) Q39E:Q38K and Q39R:Q38E, respectively, (iv) Q39E:Q38K and Q39R:Q38D, respectively, (v) Q39E:Q38K and Q39E:Q38K, respectively, (vi) Q39E:Q38K and Q39E:Q38R, respectively, (vii) Q39E:Q38K and Q39D:Q38K, respectively, (viii) Q39E:Q38K and Q39D:Q38R, respectively, (ix) Q39E:Q38R and Q39K:Q38E, respectively, (x) Q39E:Q38R and Q39K:Q38D, respectively, (xi) Q39E:Q38R and Q39R:Q38E, respectively, (xii) Q39E:Q38R and Q39R:Q38D, respectively, (xiii) Q39E:Q38R and Q39E:Q38K, respectively, (xiv) Q39E:Q38R and Q39E:Q38R, respectively, (xv) Q39E:Q38R and Q39D:Q38K, respectively, (xvi) Q39E:Q38R and Q39D:Q38R, respectively, (xvii) Q39D:Q38K and Q39K:Q38E, respectively, (xviii) Q39D:Q38K and Q39K:Q38D, respectively, (xix) Q39D:Q38K and Q39R:Q38E, respectively, (xx) Q39D:Q38K and Q39R:Q38D, respectively, (xxi) Q39D:Q38K and Q39E:Q38K, respectively, (xxii) Q39D:Q38K and Q39E:Q38R, respectively, (xxiii) Q39D:Q38K and Q39D:Q38K, respectively, (xxiv) Q39D:Q38K and Q39D:Q38R, respectively, (xxv) Q39D:Q38R and Q39K:Q38E, respectively, (xxvi) Q39D:Q38R and Q39K:Q38D, respectively, (xxvii) Q39D:Q38R and Q39R:Q38E, respectively, (xxviii) Q39D:Q38R and Q39R:Q38D, respectively, (xxix) Q39D:Q38R and Q39E:Q38K, respectively, (xxx) Q39D:Q38R and Q39E:Q38R, respectively, (xxxi) Q39D:Q38R and Q39D:Q38K, respectively, (xxxii) Q39D:Q38R and Q39D:Q38R, respectively, (xxxiii) Q39K:Q38E and Q39K:Q38E, respectively, (xxxiv) Q39K:Q38E and Q39K:Q38D, respectively, (xxxv) Q39K:Q38E and Q39R:Q38E, respectively, (xxxvi) Q39K:Q38E and Q39R:Q38D, respectively, (xxxvii) Q39K:Q38E and Q39E:Q38K, respectively, (xxxviii) Q39K:Q38E and Q39E:Q38R, respectively, (xxxix) Q39K:Q38E and Q39D:Q38K, respectively, (xl) Q39K:Q38E and Q39D:Q38R, respectively, (xli) Q39R:Q38E and Q39K:Q38E, respectively, (xlii) Q39R:Q38E and Q39K:Q38D, respectively, (xliii) Q39R:Q38E and Q39R:Q38E, respectively, (xliv) Q39R:Q38E and Q39R:Q38D, respectively, (xlv) Q39R:Q38E and Q39E:Q38K, respectively, (xlvi) Q39R:Q38E and Q39E:Q38R, respectively, (xlvii) Q39R:Q38E and Q39D:Q38K, respectively, (xlviii) Q39R:Q38E and Q39D:Q38R, respectively, (xlix) Q39K:Q38D and Q39K:Q38E, respectively, (1) Q39K:Q38D and Q39K:Q38D, respectively, (li) Q39K:Q38D and Q39R:Q38E, respectively, (lii) Q39K:Q38D and Q39R:Q38D, respectively, (liii) Q39K:Q38D and Q39E:Q38K, respectively, (liv) Q39K:Q38D and Q39E:Q38R, respectively, (lv) Q39K:Q38D and Q39D:Q38K, respectively, (lvi) Q39K:Q38D and Q39D:Q38R, respectively, (lvii) Q39R:Q38D and Q39K:Q38E, respectively, (lviii) Q39R:Q38D and Q39K:Q38D, respectively, (lix) Q39R:Q38D and Q39R:Q38E, respectively, (lx) Q39R:Q38D and Q39R:Q38D, respectively, (lxi) Q39R:Q38D and Q39E:Q38K, respectively, (lxii) Q39R:Q38D and Q39E:Q38R, respectively, (lxiii) Q39R:Q38D and Q39D:Q38K, respectively, and (lxiv) Q39R:Q38D and Q39D:Q38R, respectively, wherein numbering is according to Kabat numbering. In some embodiments, the first set of VH:VL electrostatic variants comprises amino acid substitutions Q39E:Q38K, and the second set of VH:VL electrostatic variants comprises amino acid substitutions Q39K:Q38E, wherein numbering is according to Kabat numbering. In other embodiments, the first set of VH:VL electrostatic variants comprises amino acid substitutions Q39K:Q38E, and the second set of VH:VL electrostatic variants comprises amino acid substitutions Q39E:Q38K, wherein numbering is according to Kabat numbering. In some embodiments, the first set of VH:VL electrostatic variants comprises any one of the sets of VH:VL electrostatic variants shown in FIG. 7 under the heading “Fab A,” and the second set of VH:VL electrostatic variants comprises the corresponding set of VH:VL electrostatic variants shown in FIG. 7 under the heading “Fab B.”

In some embodiments, the first monomer and the second monomer comprise the first set of VH:VL electrostatic variants, and the third monomer and the fourth monomer comprise the second set of VH:VL electrostatic variants.

In some embodiments, the multimeric protein has antigen binding affinity substantially equivalent to a corresponding multimeric protein lacking the amino acid substitutions at the VH:VL interface(s).

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

### 5. CH1:CL (Electrostatic)+CH1:CL (Steric)+VH:VL:

In some embodiments, a multimeric protein is provided, wherein the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein: (i) VL2 is a second variable light domain, (ii) the second variable heavy domain and the second variable light domain form a second antigen binding domain that binds to a second target antigen of interest (or, in some other embodiments, to the first target antigen of interest), (iii) the first monomer binds to the second monomer by CH1-CL1 dimerization and VH1-VL1 association, and (iv) the third monomer binds to the fourth monomer by CH1-CL2 dimerization and VH2-VL2 association.

In some embodiments, the multimeric protein further comprises a set of CH1:CL electrostatic variants, a set of CH1:CL steric variants, and a set of VH:VL electrostatic variants.

In some embodiments, the set of CH1:CL electrostatic variants comprises amino acid substitutions at amino acid residues K213/K218:D122/E123, wherein numbering is according to EU numbering. For the sake of clarity, while residue K218 can be considered to be a part of the hinge region according to EU numbering, it is expressly contemplated that, when discussed in the context of a CH1:CL interface variant (such as, for example, a CH1:CL electrostatic variant) or a set of CH1:CL interface variants (such as, for example, a set of CH1:CL electrostatic variants), residue K218 is considered to be a part of the CH1 domain, even in instances where amino acid substitutions of this residue are characterized as “according to EU numbering.” In some embodiments, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering. In some embodiments, the set of CH1:CL electrostatic variants comprises amino acid substitutions K213E/K218D:D122K/E123K, wherein numbering is according to EU numbering. In some embodiments, the set of CH1:CL electrostatic variants comprises any one of the sets of CH1:CL electrostatic variants shown in FIG. 3.

In some embodiments, the set of CH1:CL steric variants comprises amino acid substitutions at amino acid residues selected from a group including: (i) A141:F118, (ii) A141:F116, and (iii) K147:S131. In some embodiments, the set of CH1:CL steric variants comprises amino acid substitutions selected from a group including: (i) A141F:F118A, (ii) A141F:F116A, and (iii) K147S:S131K, wherein numbering is according to EU numbering. In some embodiments, the set of CH1:CL steric variants comprises any one of the sets of CH1:CL steric variants shown in FIG. 5.

In some embodiments, the set of VH:VL electrostatic variants comprise amino acid substitutions at amino acid residues Q39:Q38, wherein numbering is according to Kabat numbering. In some embodiments, the set of VH:VL electrostatic variants comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. In some embodiments, the set of VH:VL electrostatic variants comprises amino acid substitutions Q39E:Q38K, wherein numbering is according to Kabat numbering. In some embodiments, the set of VH:VL electrostatic variants comprises any one of the sets of VH:VL electrostatic variants shown in FIG. 7.

In some embodiments, the first monomer and the second monomer comprise the set of CH1:CL electrostatic variants, the set of CH1:CL steric variants, and the set of VH:VL electrostatic variants (and the third monomer and the fourth monomer do not comprise interface variants). In some embodiments of the first monomer and the second monomer, the set of CH1:CL electrostatic variants is selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering; the set of CH1:CL steric variants is selected from a group including: (i) A141F:F118A, (ii) A141F:F116A, and (iii) K147S:S131K, wherein numbering is according to EU numbering, and the set of VH:VL electrostatic variants is selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. Any one of the sets of CH1:CL electrostatic variants listed in (i)-(xvi), any one of the sets of CH1:CL steric variants listed in (i)-(iii), and any one of the sets of VH:VL electrostatic variants listed in (i)-(viii) can be found in the first and second monomers of the multimeric protein described herein.

In other embodiments, the third monomer and the fourth monomer comprise the set of CH1:CL electrostatic variants, the set of CH1:CL steric variants, and the set of VH:VL electrostatic variants (and the first monomer and the second monomer do not comprise interface variants). In some embodiments of the third monomer and the fourth monomer, the set of CH1:CL electrostatic variants is selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering; the set of CH1:CL steric variants is selected from a group including: (i) A141F:F118A, (ii) A141F:F116A, and (iii) K147S:S131K, wherein numbering is according to EU numbering, and the set of VH:VL electrostatic variants is selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. Any one of the sets of CH1:CL electrostatic variants listed in (i)-(xvi), any one of the sets of CH1:CL steric variants listed in (i)-(iii), and any one of the sets of VH:VL electrostatic variants listed in (i)-(viii) can be found in the third and fourth monomers of the multimeric protein described herein.

In some embodiments, the first monomer and the second monomer comprise a set of CH1:CL electrostatic variants provided in the list above and a set of CH1:CL steric variants provided in the list above, and the third monomer and the fourth monomer comprise a set of VH:VL electrostatic variants provided in the list above. In other embodiments, the first monomer and the second monomer comprise a set of VH:VL electrostatic variants provided in the list above, and the third monomer and the fourth monomer comprise a set of CH1:CL electrostatic variants provided in the list above and a set of CH1:CL steric variants provided in the list above.

In some embodiments, the first monomer and the second monomer comprise a set of CH1:CL electrostatic variants provided in the list above and a set of VH:VL electrostatic variants provided in the list above, and the third monomer and the fourth monomer comprise a set of CH1:CL steric variants provided in the list above. In other embodiments, the first monomer and the second monomer comprise a set of CH1:CL steric variants, and the third monomer and the fourth monomer comprise a set of CH1:CL electrostatic variants provided in the list above and a set of VH:VL electrostatic variants provided in the list above.

In some embodiments, the first monomer and the second monomer comprise a set of CH1:CL steric variants provided in the list above and a set of VH:VL steric variants provided in the list above, and the third monomer and the fourth monomer comprise a set of CH1:CL electrostatic variants provided in the list above. In other embodiments, the first monomer and the second monomer comprise a set of CH1:CL electrostatic variants provided in the list above, and the third monomer and the fourth monomer comprise a set of CH1:CL steric variants provided in the list above and a set of VH:VL steric variants provided in the list above.

In some embodiments, the multimeric protein has antigen binding affinity substantially equivalent to a corresponding multimeric protein lacking the amino acid substitutions at the VH:VL interface and the CH1:CL interfaces.

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

In some embodiments, a multimeric protein is provided, wherein the multimeric protein comprises: (a) a first monomer comprising a VH-CH1 domain, wherein VH is a variable heavy domain; and (b) a second monomer comprising a VL-CL domain, wherein VL is a variable light domain, and wherein the variable heavy domain and the variable light domain form an antigen binding domain that binds to a target antigen of interest, and the first monomer binds to the second monomer by CH1-CL dimerization and VH-VL association.

In some embodiments, the multimeric protein further comprises a set of CH1:CL electrostatic variants, a set of CH1:CL steric variants, and a set of VH:VL electrostatic variants.

In some embodiments, the set of CH1:CL electrostatic variants comprises amino acid substitutions at amino acid residues K213/K218:D122/E123, wherein numbering is according to EU numbering. In some embodiments, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering. In some embodiments, the set of CH1:CL electrostatic variants comprises amino acid substitutions K213E/K218D:D122K/E123K, wherein numbering is according to EU numbering. In some embodiments, the set of CH1:CL electrostatic variants comprises any one of the sets of CH1:CL electrostatic variants shown in FIG. 3.

In some embodiments, the set of CH1:CL steric variants comprises amino acid substitutions at amino acid residues selected from a group including: (i) A141:F118, (ii) A141:F116, and (iii) K147:S131. In some embodiments, the set of CH1:CL steric variants comprises amino acid substitutions selected from a group including: (i) A141F:F118A, (ii) A141F:F116A, and (iii) K147S:S131K, wherein numbering is according to EU numbering. In some embodiments, the set of CH1:CL steric variants comprises any one of the sets of CH1:CL steric variants shown in FIG. 5.

In some embodiments, the set of VH:VL electrostatic variants comprise amino acid substitutions at amino acid residues Q39:Q38, wherein numbering is according to Kabat numbering. In some embodiments, the set of VH:VL electrostatic variants comprise amino acid substitutions selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering. In some embodiments, the set of VH:VL electrostatic variants comprises amino acid substitutions Q39E:Q38K, wherein numbering is according to Kabat numbering. In some embodiments, the set of VH:VL electrostatic variants comprises any one of the sets of VH:VL electrostatic variants shown in FIG. 7.

In some embodiments of the first monomer and the second monomer, the set of CH1:CL electrostatic variants is selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering; the set of CH1:CL steric variants is selected from a group including: (i) A141F:F118A, (ii) A141F:F116A, and (iii) K147S:S131K, wherein numbering is according to EU numbering, and the set of VH:VL electrostatic variants is selected from a group including: (i) Q39E:Q38K, (ii) Q39E:Q38R, (iii) Q39D:Q38K, (iv) Q39D:Q38R, (v) Q39K:Q38E, (vi) Q39R:Q38E, (vii) Q39K:Q38D, and (viii) Q39R:Q38D, wherein numbering is according to Kabat numbering.

In some embodiments, the multimeric protein has antigen binding affinity substantially equivalent to a corresponding multimeric protein lacking the amino acid substitutions at the VH:VL interface and the CH1:CL interfaces.

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

### 6. CH1:CL (Electrostatic)+VH:VL (1” Set)+VH:VL (2^{nd} Set):

In some embodiments, a multimeric protein is provided, wherein the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein: (i) VL2 is a second variable light domain, (ii) the second variable heavy domain and the second variable light domain form a second antigen binding domain that binds to a second target antigen of interest (or, in some other embodiments, to the first target antigen of interest), (iii) the first monomer binds to the second monomer by CH1-CL1 dimerization and VH1-VL1 association, and (iv) the third monomer binds to the fourth monomer by CH1-CL2 dimerization and VH2-VL2 association.

In some embodiments, the multimeric protein further comprises a set of CH1:CL electrostatic variants, a first set of VH:VL electrostatic variants, and a second set of VH:VL electrostatic variants.

In some embodiments, the set of CH1:CL electrostatic variants comprises amino acid substitutions at amino acid residues K213/K218:D122/E123, wherein numbering is according to EU numbering. For the sake of clarity, while residue K218 can be considered to be a part of the hinge region according to EU numbering, it is expressly contemplated that, when discussed in the context of a CH1:CL interface variant (such as, for example, a CH1:CL electrostatic variant) or a set of CH1:CL interface variants (such as, for example, a set of CH1:CL electrostatic variants), residue K218 is considered to be a part of the CH1 domain, even in instances where amino acid substitutions of this residue are characterized as “according to EU numbering.” In some embodiments, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering. In some embodiments, the set of CH1:CL electrostatic variants comprises amino acid substitutions K213E/K218D:D122K/E123K, wherein numbering is according to EU numbering. In some embodiments, the set of CH1:CL electrostatic variants comprises any one of the sets of CH1:CL electrostatic variants shown in FIG. 3.

In some embodiments, the first set of VH:VL electrostatic variants and the second set of VH:VL electrostatic variants comprise amino acid substitutions selected from a group including: (i) Q39E:Q38K and Q39K:Q38E, respectively, (ii) Q39E:Q38K and Q39K:Q38D, respectively, (iii) Q39E:Q38K and Q39R:Q38E, respectively, (iv) Q39E:Q38K and Q39R:Q38D, respectively, (v) Q39E:Q38K and Q39E:Q38K, respectively, (vi) Q39E:Q38K and Q39E:Q38R, respectively, (vii) Q39E:Q38K and Q39D:Q38K, respectively, (viii) Q39E:Q38K and Q39D:Q38R, respectively, (ix) Q39E:Q38R and Q39K:Q38E, respectively, (x) Q39E:Q38R and Q39K:Q38D, respectively, (xi) Q39E:Q38R and Q39R:Q38E, respectively, (xii) Q39E:Q38R and Q39R:Q38D, respectively, (xiii) Q39E:Q38R and Q39E:Q38K, respectively, (xiv) Q39E:Q38R and Q39E:Q38R, respectively, (xv) Q39E:Q38R and Q39D:Q38K, respectively, (xvi) Q39E:Q38R and Q39D:Q38R, respectively, (xvii) Q39D:Q38K and Q39K:Q38E, respectively, (xviii) Q39D:Q38K and Q39K:Q38D, respectively, (xix) Q39D:Q38K and Q39R:Q38E, respectively, (xx) Q39D:Q38K and Q39R:Q38D, respectively, (xxi) Q39D:Q38K and Q39E:Q38K, respectively, (xxii) Q39D:Q38K and Q39E:Q38R, respectively, (xxiii) Q39D:Q38K and Q39D:Q38K, respectively, (xxiv) Q39D:Q38K and Q39D:Q38R, respectively, (xxv) Q39D:Q38R and Q39K:Q38E, respectively, (xxvi) Q39D:Q38R and Q39K:Q38D, respectively, (xxvii) Q39D:Q38R and Q39R:Q38E, respectively, (xxviii) Q39D:Q38R and Q39R:Q38D, respectively, (xxix) Q39D:Q38R and Q39E:Q38K, respectively, (xxx) Q39D:Q38R and Q39E:Q38R, respectively, (xxxi) Q39D:Q38R and Q39D:Q38K, respectively, (xxxii) Q39D:Q38R and Q39D:Q38R, respectively, (xxxiii) Q39K:Q38E and Q39K:Q38E, respectively, (xxxiv) Q39K:Q38E and Q39K:Q38D, respectively, (xxxv) Q39K:Q38E and Q39R:Q38E, respectively, (xxxvi) Q39K:Q38E and Q39R:Q38D, respectively, (xxxvii) Q39K:Q38E and Q39E:Q38K, respectively, (xxxviii) Q39K:Q38E and Q39E:Q38R, respectively, (xxxix) Q39K:Q38E and Q39D:Q38K, respectively, (xl) Q39K:Q38E and Q39D:Q38R, respectively, (xli) Q39R:Q38E and Q39K:Q38E, respectively, (xlii) Q39R:Q38E and Q39K:Q38D, respectively, (xliii) Q39R:Q38E and Q39R:Q38E, respectively, (xliv) Q39R:Q38E and Q39R:Q38D, respectively, (xlv) Q39R:Q38E and Q39E:Q38K, respectively, (xlvi) Q39R:Q38E and Q39E:Q38R, respectively, (xlvii) Q39R:Q38E and Q39D:Q38K, respectively, (xlviii) Q39R:Q38E and Q39D:Q38R, respectively, (xlix) Q39K:Q38D and Q39K:Q38E, respectively, (1) Q39K:Q38D and Q39K:Q38D, respectively, (li) Q39K:Q38D and Q39R:Q38E, respectively, (lii) Q39K:Q38D and Q39R:Q38D, respectively, (liii) Q39K:Q38D and Q39E:Q38K, respectively, (liv) Q39K:Q38D and Q39E:Q38R, respectively, (lv) Q39K:Q38D and Q39D:Q38K, respectively, (lvi) Q39K:Q38D and Q39D:Q38R, respectively, (lvii) Q39R:Q38D and Q39K:Q38E, respectively, (lviii) Q39R:Q38D and Q39K:Q38D, respectively, (lix) Q39R:Q38D and Q39R:Q38E, respectively, (lx) Q39R:Q38D and Q39R:Q38D, respectively, (lxi) Q39R:Q38D and Q39E:Q38K, respectively, (lxii) Q39R:Q38D and Q39E:Q38R, respectively, (lxiii) Q39R:Q38D and Q39D:Q38K, respectively, and (lxiv) Q39R:Q38D and Q39D:Q38R, respectively, wherein numbering is according to Kabat numbering. In some embodiments, the first set of VH:VL electrostatic variants comprises amino acid substitutions Q39E:Q38K, and the second set of VH:VL electrostatic variants comprises amino acid substitutions Q39K:Q38E, wherein numbering is according to Kabat numbering. In other embodiments, the first set of VH:VL electrostatic variants comprises amino acid substitutions Q39K:Q38E, and the second set of VH:VL electrostatic variants comprises amino acid substitutions Q39E:Q38K, wherein numbering is according to Kabat numbering. In some embodiments, the first set of VH:VL electrostatic variants comprises any one of the sets of VH:VL electrostatic variants shown in FIG. 7 under the heading “Fab A,” and the second set of VH:VL electrostatic variants comprises the corresponding set of VH:VL electrostatic variants shown in FIG. 7 under the heading “Fab B.”

In some embodiments, the first monomer and the second monomer comprise a set of CH1:CL electrostatic variants provided in the sections above and a first set of VH:VL electrostatic variants provided in the sections above. In other embodiments, the first monomer and the second monomer comprise a first set of VH:VL electrostatic variants provided in the sections above, and the third monomer and the fourth monomer comprise a set of CH1:CL electrostatic variants provided in the sections above and a second set of VH:VL electrostatic variants provided in the sections above.

In some embodiments, the multimeric protein has antigen binding affinity substantially equivalent to a corresponding multimeric protein lacking the amino acid substitutions at the VH:VL interfaces and the CH1:CL interface.

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

7. CH1:CL (Steric)+VH:VL (1^{st} Set)+VH:VL (2^{nd} Set):

In some embodiments, a multimeric protein is provided, wherein the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein: (i) VL2 is a second variable light domain, (ii) the second variable heavy domain and the second variable light domain form a second antigen binding domain that binds to a second target antigen of interest (or, in some other embodiments, to the first target antigen of interest), (iii) the first monomer binds to the second monomer by CH1-CL1 dimerization and VH1-VL1 association, and (iv) the third monomer binds to the fourth monomer by CH1-CL2 dimerization and VH2-VL2 association.

In some embodiments, the multimeric protein further comprises a set of CH1:CL steric variants, a first set of VH:VL electrostatic variants, and a second set of VH:VL electrostatic variants.

In some embodiments, the set of CH1:CL steric variants comprises amino acid substitutions at amino acid residues selected from a group including: (i) A141:F118, (ii) A141:F116, and (iii) K147:S131. In some embodiments, the set of CH1:CL steric variants comprises amino acid substitutions selected from a group including: (i) A141F:F118A, (ii) A141F:F116A, and (iii) K147S:S131K, wherein numbering is according to EU numbering. In some embodiments, the set of CH1:CL steric variants comprises any one of the sets of CH1:CL steric variants shown in FIG. 5.

In some embodiments, the first set of VH:VL electrostatic variants and the second set of VH:VL electrostatic variants comprise amino acid substitutions selected from a group including: (i) Q39E:Q38K and Q39K:Q38E, respectively, (ii) Q39E:Q38K and Q39K:Q38D, respectively, (iii) Q39E:Q38K and Q39R:Q38E, respectively, (iv) Q39E:Q38K and Q39R:Q38D, respectively, (v) Q39E:Q38K and Q39E:Q38K, respectively, (vi) Q39E:Q38K and Q39E:Q38R, respectively, (vii) Q39E:Q38K and Q39D:Q38K, respectively, (viii) Q39E:Q38K and Q39D:Q38R, respectively, (ix) Q39E:Q38R and Q39K:Q38E, respectively, (x) Q39E:Q38R and Q39K:Q38D, respectively, (xi) Q39E:Q38R and Q39R:Q38E, respectively, (xii) Q39E:Q38R and Q39R:Q38D, respectively, (xiii) Q39E:Q38R and Q39E:Q38K, respectively, (xiv) Q39E:Q38R and Q39E:Q38R, respectively, (xv) Q39E:Q38R and Q39D:Q38K, respectively, (xvi) Q39E:Q38R and Q39D:Q38R, respectively, (xvii) Q39D:Q38K and Q39K:Q38E, respectively, (xviii) Q39D:Q38K and Q39K:Q38D, respectively, (xix) Q39D:Q38K and Q39R:Q38E, respectively, (xx) Q39D:Q38K and Q39R:Q38D, respectively, (xxi) Q39D:Q38K and Q39E:Q38K, respectively, (xxii) Q39D:Q38K and Q39E:Q38R, respectively, (xxiii) Q39D:Q38K and Q39D:Q38K, respectively, (xxiv) Q39D:Q38K and Q39D:Q38R, respectively, (xxv) Q39D:Q38R and Q39K:Q38E, respectively, (xxvi) Q39D:Q38R and Q39K:Q38D, respectively, (xxvii) Q39D:Q38R and Q39R:Q38E, respectively, (xxviii) Q39D:Q38R and Q39R:Q38D, respectively, (xxix) Q39D:Q38R and Q39E:Q38K, respectively, (xxx) Q39D:Q38R and Q39E:Q38R, respectively, (xxxi) Q39D:Q38R and Q39D:Q38K, respectively, (xxxii) Q39D:Q38R and Q39D:Q38R, respectively, (xxxiii) Q39K:Q38E and Q39K:Q38E, respectively, (xxxiv) Q39K:Q38E and Q39K:Q38D, respectively, (xxxv) Q39K:Q38E and Q39R:Q38E, respectively, (xxxvi) Q39K:Q38E and Q39R:Q38D, respectively, (xxxvii) Q39K:Q38E and Q39E:Q38K, respectively, (xxxviii) Q39K:Q38E and Q39E:Q38R, respectively, (xxxix) Q39K:Q38E and Q39D:Q38K, respectively, (xl) Q39K:Q38E and Q39D:Q38R, respectively, (xli) Q39R:Q38E and Q39K:Q38E, respectively, (xlii) Q39R:Q38E and Q39K:Q38D, respectively, (xliii) Q39R:Q38E and Q39R:Q38E, respectively, (xliv) Q39R:Q38E and Q39R:Q38D, respectively, (xlv) Q39R:Q38E and Q39E:Q38K, respectively, (xlvi) Q39R:Q38E and Q39E:Q38R, respectively, (xlvii) Q39R:Q38E and Q39D:Q38K, respectively, (xlviii) Q39R:Q38E and Q39D:Q38R, respectively, (xlix) Q39K:Q38D and Q39K:Q38E, respectively, (1) Q39K:Q38D and Q39K:Q38D, respectively, (li) Q39K:Q38D and Q39R:Q38E, respectively, (lii) Q39K:Q38D and Q39R:Q38D, respectively, (liii) Q39K:Q38D and Q39E:Q38K, respectively, (liv) Q39K:Q38D and Q39E:Q38R, respectively, (lv) Q39K:Q38D and Q39D:Q38K, respectively, (lvi) Q39K:Q38D and Q39D:Q38R, respectively, (lvii) Q39R:Q38D and Q39K:Q38E, respectively, (lviii) Q39R:Q38D and Q39K:Q38D, respectively, (lix) Q39R:Q38D and Q39R:Q38E, respectively, (lx) Q39R:Q38D and Q39R:Q38D, respectively, (lxi) Q39R:Q38D and Q39E:Q38K, respectively, (lxii) Q39R:Q38D and Q39E:Q38R, respectively, (lxiii) Q39R:Q38D and Q39D:Q38K, respectively, and (lxiv) Q39R:Q38D and Q39D:Q38R, respectively, wherein numbering is according to Kabat numbering. In some embodiments, the first set of VH:VL electrostatic variants comprises amino acid substitutions Q39E:Q38K, and the second set of VH:VL electrostatic variants comprises amino acid substitutions Q39K:Q38E, wherein numbering is according to Kabat numbering. In other embodiments, the first set of VH:VL electrostatic variants comprises amino acid substitutions Q39K:Q38E, and the second set of VH:VL electrostatic variants comprises amino acid substitutions Q39E:Q38K, wherein numbering is according to Kabat numbering. In some embodiments, the first set of VH:VL electrostatic variants comprises any one of the sets of VH:VL electrostatic variants shown in FIG. 7 under the heading “Fab A,” and the second set of VH:VL electrostatic variants comprises the corresponding set of VH:VL electrostatic variants shown in FIG. 7 under the heading “Fab B.”

In some embodiments, the first monomer and the second monomer comprise a set of CH1:CL steric variants provided in the sections above and a first set of VH:VL electrostatic variants provided in the sections above. In other embodiments, the first monomer and the second monomer comprise a first set of VH:VL electrostatic variants provided in the sections above, and the third monomer and the fourth monomer comprise a set of CH1:CL steric variants provided in the sections above and a second set of VH:VL electrostatic variants provided in the sections above.

In some embodiments, the multimeric protein has antigen binding affinity substantially equivalent to a corresponding multimeric protein lacking the amino acid substitutions at the VH:VL interfaces and the CH1:CL interface.

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

8. CH1:CL (Electrostatic)+CHL1:CL (Steric)+VH:VL (1^{st} Set)+VH:VL (2^{nd} Set):

In some embodiments, a multimeric protein is provided, wherein the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein: (i) VL2 is a second variable light domain, (ii) the second variable heavy domain and the second variable light domain form a second antigen binding domain that binds to a second target antigen of interest (or, in some other embodiments, to the first target antigen of interest), (iii) the first monomer binds to the second monomer by CH1-CL1 dimerization and VH1-VL1 association, and (iv) the third monomer binds to the fourth monomer by CH1-CL2 dimerization and VH2-VL2 association.

In some embodiments, the multimeric protein further comprises a set of CH1:CL electrostatic variants, a set of CH1:CL steric variants, a first set of VH:VL electrostatic variants, and a second set of VH:VL electrostatic variants.

In some embodiments, the set of CH1:CL electrostatic variants comprises amino acid substitutions at amino acid residues K213/K218:D122/E123, wherein numbering is according to EU numbering. For the sake of clarity, while residue K218 can be considered to be a part of the hinge region according to EU numbering, it is expressly contemplated that, when discussed in the context of a CH1:CL interface variant (such as, for example, a CH1:CL electrostatic variant) or a set of CH1:CL interface variants (such as, for example, a set of CH1:CL electrostatic variants), residue K218 is considered to be a part of the CH1 domain, even in instances where amino acid substitutions of this residue are characterized as “according to EU numbering.” In some embodiments, the set of CH1:CL electrostatic variants comprises amino acid substitutions selected from a group including: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering. In some embodiments, the set of CH1:CL electrostatic variants comprises amino acid substitutions K213E/K218D:D122K/E123K, wherein numbering is according to EU numbering. In some embodiments, the set of CH1:CL electrostatic variants comprises any one of the sets of CH1:CL electrostatic variants shown in FIG. 3.

In some embodiments, the set of CH1:CL steric variants comprises amino acid substitutions at amino acid residues selected from a group including: (i) A141:F118, (ii) A141:F116, and (iii) K147:S131. In some embodiments, the set of CH1:CL steric variants comprises amino acid substitutions selected from a group including: (i) A141F:F118A, (ii) A141F:F116A, and (iii) K147S:S131K, wherein numbering is according to EU numbering. In some embodiments, the set of CH1:CL steric variants comprises any one of the sets of CH1:CL steric variants shown in FIG. 5.

In some embodiments, the first set of VH:VL electrostatic variants and the second set of VH:VL electrostatic variants comprise amino acid substitutions selected from a group including: (i) Q39E:Q38K and Q39K:Q38E, respectively, (ii) Q39E:Q38K and Q39K:Q38D, respectively, (iii) Q39E:Q38K and Q39R:Q38E, respectively, (iv) Q39E:Q38K and Q39R:Q38D, respectively, (v) Q39E:Q38K and Q39E:Q38K, respectively, (vi) Q39E:Q38K and Q39E:Q38R, respectively, (vii) Q39E:Q38K and Q39D:Q38K, respectively, (viii) Q39E:Q38K and Q39D:Q38R, respectively, (ix) Q39E:Q38R and Q39K:Q38E, respectively, (x) Q39E:Q38R and Q39K:Q38D, respectively, (xi) Q39E:Q38R and Q39R:Q38E, respectively, (xii) Q39E:Q38R and Q39R:Q38D, respectively, (xiii) Q39E:Q38R and Q39E:Q38K, respectively, (xiv) Q39E:Q38R and Q39E:Q38R, respectively, (xv) Q39E:Q38R and Q39D:Q38K, respectively, (xvi) Q39E:Q38R and Q39D:Q38R, respectively, (xvii) Q39D:Q38K and Q39K:Q38E, respectively, (xviii) Q39D:Q38K and Q39K:Q38D, respectively, (xix) Q39D:Q38K and Q39R:Q38E, respectively, (xx) Q39D:Q38K and Q39R:Q38D, respectively, (xxi) Q39D:Q38K and Q39E:Q38K, respectively, (xxii) Q39D:Q38K and Q39E:Q38R, respectively, (xxiii) Q39D:Q38K and Q39D:Q38K, respectively, (xxiv) Q39D:Q38K and Q39D:Q38R, respectively, (xxv) Q39D:Q38R and Q39K:Q38E, respectively, (xxvi) Q39D:Q38R and Q39K:Q38D, respectively, (xxvii) Q39D:Q38R and Q39R:Q38E, respectively, (xxviii) Q39D:Q38R and Q39R:Q38D, respectively, (xxix) Q39D:Q38R and Q39E:Q38K, respectively, (xxx) Q39D:Q38R and Q39E:Q38R, respectively, (xxxi) Q39D:Q38R and Q39D:Q38K, respectively, (xxxii) Q39D:Q38R and Q39D:Q38R, respectively, (xxxiii) Q39K:Q38E and Q39K:Q38E, respectively, (xxxiv) Q39K:Q38E and Q39K:Q38D, respectively, (xxxv) Q39K:Q38E and Q39R:Q38E, respectively, (xxxvi) Q39K:Q38E and Q39R:Q38D, respectively, (xxxvii) Q39K:Q38E and Q39E:Q38K, respectively, (xxxviii) Q39K:Q38E and Q39E:Q38R, respectively, (xxxix) Q39K:Q38E and Q39D:Q38K, respectively, (xl) Q39K:Q38E and Q39D:Q38R, respectively, (xli) Q39R:Q38E and Q39K:Q38E, respectively, (xlii) Q39R:Q38E and Q39K:Q38D, respectively, (xliii) Q39R:Q38E and Q39R:Q38E, respectively, (xliv) Q39R:Q38E and Q39R:Q38D, respectively, (xlv) Q39R:Q38E and Q39E:Q38K, respectively, (xlvi) Q39R:Q38E and Q39E:Q38R, respectively, (xlvii) Q39R:Q38E and Q39D:Q38K, respectively, (xlviii) Q39R:Q38E and Q39D:Q38R, respectively, (xlix) Q39K:Q38D and Q39K:Q38E, respectively, (1) Q39K:Q38D and Q39K:Q38D, respectively, (li) Q39K:Q38D and Q39R:Q38E, respectively, (lii) Q39K:Q38D and Q39R:Q38D, respectively, (liii) Q39K:Q38D and Q39E:Q38K, respectively, (liv) Q39K:Q38D and Q39E:Q38R, respectively, (lv) Q39K:Q38D and Q39D:Q38K, respectively, (lvi) Q39K:Q38D and Q39D:Q38R, respectively, (lvii) Q39R:Q38D and Q39K:Q38E, respectively, (lviii) Q39R:Q38D and Q39K:Q38D, respectively, (lix) Q39R:Q38D and Q39R:Q38E, respectively, (lx) Q39R:Q38D and Q39R:Q38D, respectively, (lxi) Q39R:Q38D and Q39E:Q38K, respectively, (lxii) Q39R:Q38D and Q39E:Q38R, respectively, (lxiii) Q39R:Q38D and Q39D:Q38K, respectively, and (lxiv) Q39R:Q38D and Q39D:Q38R, respectively, wherein numbering is according to Kabat numbering. In some embodiments, the first set of VH:VL electrostatic variants comprises amino acid substitutions Q39E:Q38K, and the second set of VH:VL electrostatic variants comprises amino acid substitutions Q39K:Q38E, wherein numbering is according to Kabat numbering. In other embodiments, the first set of VH:VL electrostatic variants comprises amino acid substitutions Q39K:Q38E, and the second set of VH:VL electrostatic variants comprises amino acid substitutions Q39E:Q38K, wherein numbering is according to Kabat numbering. In some embodiments, the first set of VH:VL electrostatic variants comprises any one of the sets of VH:VL electrostatic variants shown in FIG. 7 under the heading “Fab A,” and the second set of VH:VL electrostatic variants comprises the corresponding set of VH:VL electrostatic variants shown in FIG. 7 under the heading “Fab B.”

In some embodiments, the first monomer and the second monomer comprise a set of CH1:CL electrostatic variants provided above, a set of CH1:CL steric variants provided above, and a first set of VH:VL electrostatic variants provided above, and the third monomer and the fourth monomer comprise a second set of VH:VL electrostatic variants provided above. In other embodiments, the first monomer and the second monomer comprise a first set of VH:VL electrostatic variants provided above, and the third monomer and the fourth monomer comprise a set of CH1:CL electrostatic variants provided above, a set of CH1:CL steric variants provided above, and a second set of VH:VL electrostatic variants provided above.

In some embodiments, a first monomer and the second monomer comprise a set of CH1:CL electrostatic variants provided above and a first set of VH:VL electrostatic variants provided above, and the third monomer and the fourth monomer comprise a set of CH1:CL steric variants provided above and a second set of VH:VL electrostatic variants provided above. In other embodiments, the first monomer and the second monomer comprise a set of CH1:CL steric variants provided above and a first set of VH:VL electrostatic variants provided above, and the third monomer and the fourth monomer comprise a set of CH1:CL electrostatic variants provided above and a second set of VH:VL electrostatic variants provided above.

In some embodiments, the multimeric protein has antigen binding affinity substantially equivalent to a corresponding multimeric protein lacking the amino acid substitutions at the VH:VL interfaces and the CH1:CL interfaces.

As will be appreciated by those in the art, any of the interface variants can also independently and optionally be combined with one or more of the following: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below.

### D. Particular Embodiments of the Invention

In an exemplary embodiment, the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain that binds to a second target antigen of interest (or, in some other exemplary embodiments, to the first target antigen of interest), wherein: (i) the first monomer and the second monomer comprise a first set of VH:VL electrostatic variants comprising amino acid substitutions Q39E:Q38K, and a set of CH1:CL electrostatic variants comprising amino acid substitutions K213E/K218D:D122K/E123K, (ii) the third monomer and the fourth monomer comprise a second set of VH:VL electrostatic variants comprising amino acid substitutions Q39K:Q38E, and a set of CH1:CL steric variants comprising amino acid substitutions A141F:F 116A, (iii) numbering of the first set and second set of VH:VL electrostatic variants is according to Kabat numbering, (iv) numbering of the set of CH1:CL electrostatic variants and the set of CH1:CL steric variants is according to EU numbering, (v) the first monomer binds to the second monomer by CH1-CL1 dimerization and VH1-VL1 association, and (vi) the third monomer binds to the fourth monomer by CH1-CL2 dimerization and VH2-VL2 association. Any of the multimeric proteins comprising one or more sets of interface variants (e.g., CH1:CL electrostatic variant, CH1:CL steric variant, VH:VL electrostatic variant) can also have one or more amino acid substitutions (or one or more sets of amino acid substitutions) in a CH2 domain and/or CH3 domain of an IgG Fc domain (e.g., a first variant IgG Fc domain, a second variant IgG Fc domain), including but not limited to: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below. Any of the multimeric proteins can also have one or more amino acid substitutions (or one or more sets of amino acid substitutions) described herein in the (IgG) Fe domains set forth in the Figures including but not limited to, FIGS. 13A-13F, 14, 15, 16, and 17 or in the heterodimeric Fc monomers set forth in the Figures including but not limited to FIGS. 18A-18D, 19A-19C, 20A-20C, 21A-21C, and 22A-22C.

In another exemplary embodiment, the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain that binds to a second target antigen of interest (or, in some other exemplary embodiments, to the first target antigen of interest), wherein: (i) the first monomer and the second monomer comprise a first set of VH:VL electrostatic variants comprising amino acid substitutions Q39E:Q38K, and a set of CH1:CL electrostatic variants comprising amino acid substitutions K213E/K218D:D122K/E123K, (ii) the third monomer and the fourth monomer comprise a second set of VH:VL electrostatic variants comprising amino acid substitutions Q39K:Q38E, and a set of CH1:CL steric variants comprising amino acid substitutions A141F:F 118A, (iii) numbering of the first set and second set of VH:VL electrostatic variants is according to Kabat numbering, (iv) numbering of the set of CH1:CL electrostatic variants and the set of CH1:CL steric variants is according to EU numbering, (v) the first monomer binds to the second monomer by CH1-CL1 dimerization and VH1-VL1 association, and (vi) the third monomer binds to the fourth monomer by CH1-CL2 dimerization and VH2-VL2 association. Any of the multimeric proteins comprising one or more sets of interface variants (e.g., CH1:CL electrostatic variant, CH1:CL steric variant, VH:VL electrostatic variant) can also have one or more amino acid substitutions (or one or more sets of amino acid substitutions) in a CH2 domain and/or CH3 domain of an IgG Fc domain (e.g., a first variant IgG Fc domain, a second variant IgG Fc domain), including but not limited to: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below. Any of the multimeric proteins can also have one or more amino acid substitutions (or one or more sets of amino acid substitutions) described herein in the (IgG) Fc domains set forth in the Figures including but not limited to, FIGS. 13A-13F, 14, 15, 16, and 17 or in the heterodimeric Fc monomers set forth in the Figures including but not limited to FIGS. 18A-18D, 19A-19C, 20A-20C, 21A-21C, and 22A-22C.

In another exemplary embodiment, the multimeric protein comprises: (a) a first monomer comprising a VH1-CH1-hinge-CH2-CH3 monomer, wherein VH1 is a first variable heavy domain and CH2-CH3 is a first variant IgG Fc domain; (b) a second monomer comprising a VL1-CL1 monomer, wherein VL1 is a first variable light domain, and wherein the first variable heavy domain and the first variable light domain form a first antigen binding domain that binds to a first target antigen of interest; (c) a third monomer comprising a VH2-CH1-hinge-CH2-CH3 monomer, wherein VH2 is a second variable heavy domain and CH2-CH3 is a second variant IgG Fc domain; and (d) a fourth monomer comprising a VL2-CL2 monomer, wherein VL2 is a second variable light domain, and wherein the second variable heavy domain and the second variable light domain form a second antigen binding domain that binds to a second target antigen of interest (or, in some other exemplary embodiments, to the first target antigen of interest), wherein: (i) the first monomer and the second monomer comprise a first set of VH:VL electrostatic variants comprising amino acid substitutions Q39E:Q38K, and a set of CH1:CL electrostatic variants comprising amino acid substitutions K213E/K218D:D122K/E123K, (ii) the third monomer and the fourth monomer comprise a second set of VH:VL electrostatic variants comprising amino acid substitutions Q39K:Q38E, and a set of CH1:CL steric variants comprising amino acid substitutions K147S:S131K, (iii) numbering of the first set and second set of VH:VL electrostatic variants is according to Kabat numbering, (iv) numbering of the set of CH1:CL electrostatic variants and the set of CH1:CL steric variants is according to EU numbering, (v) the first monomer binds to the second monomer by CH1-CL1 dimerization and VH1-VL1 association, and (vi) the third monomer binds to the fourth monomer by CH1-CL2 dimerization and VH2-VL2 association. Any of the multimeric proteins comprising one or more sets of interface variants (e.g., CH1:CL electrostatic variant, CH1:CL steric variant, VH:VL electrostatic variant) can also have one or more amino acid substitutions (or one or more sets of amino acid substitutions) in a CH2 domain and/or CH3 domain of an IgG Fc domain (e.g., a first variant IgG Fc domain, a second variant IgG Fc domain), including but not limited to: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below. Any of the multimeric proteins can also have one or more amino acid substitutions (or one or more sets of amino acid substitutions) described herein in the (IgG) Fc domains set forth in the Figures including but not limited to, FIGS. 13A-13F, 14, 15, 16, and 17 or in the heterodimeric Fc monomers set forth in the Figures including but not limited to FIGS. 18A-18D, 19A-19C, 20A-20C, 21A-21C, and 22A-22C.

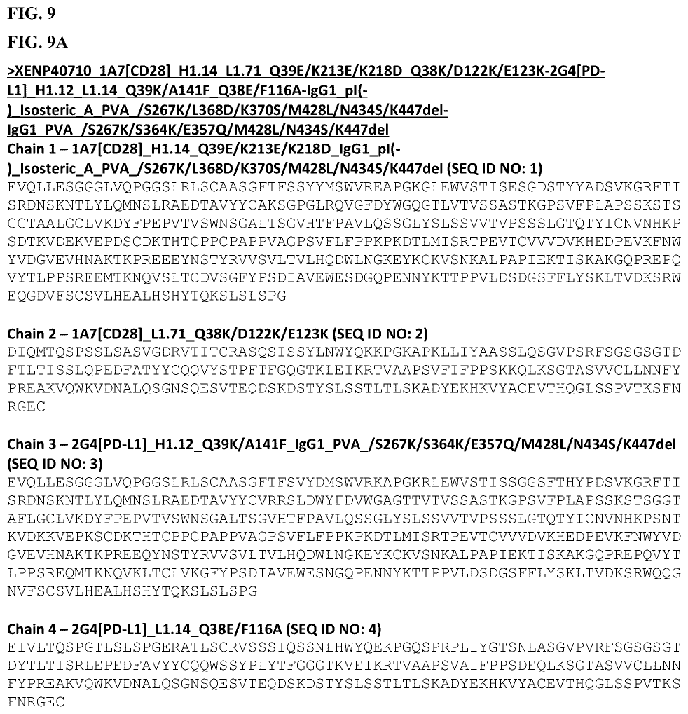

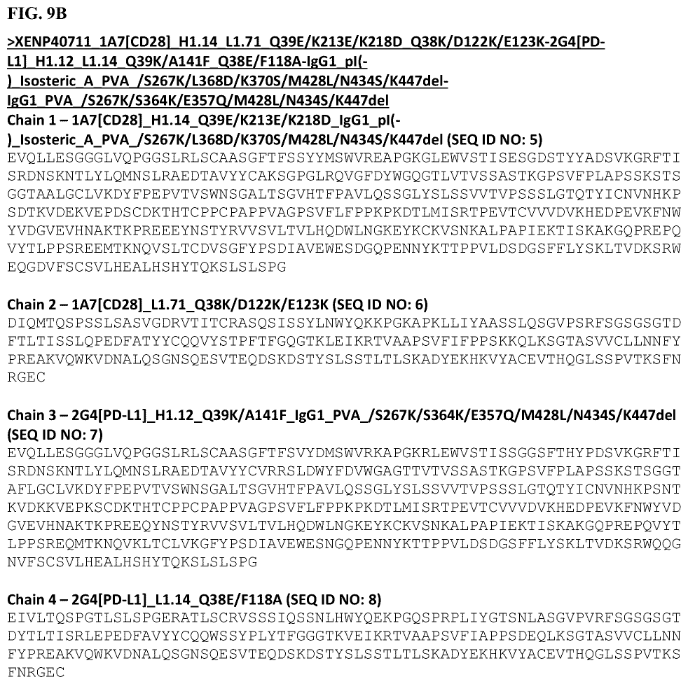

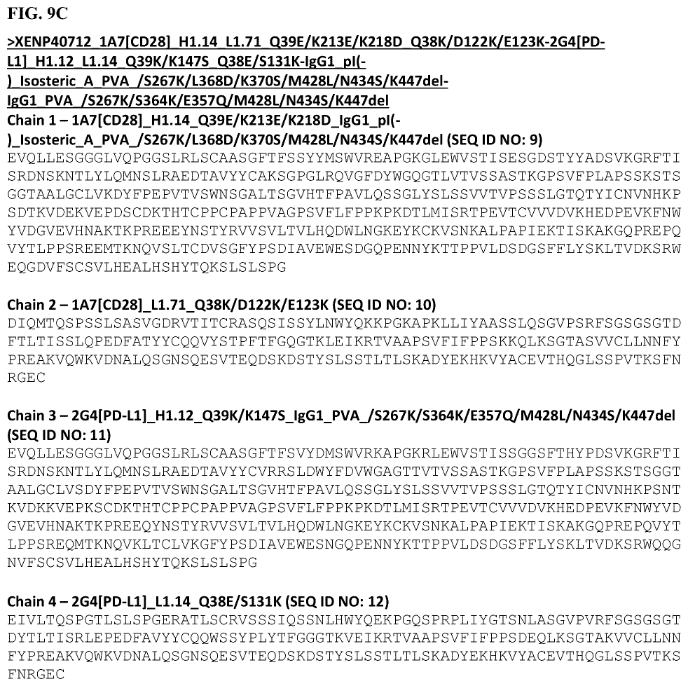

In another exemplary embodiment, the multimeric protein comprises: (a) a first monomer comprising SEQ ID NO: 1; (b) a second monomer comprising SEQ ID NO: 2; (c) a third monomer comprising SEQ ID NO: 3; and (d) a fourth monomer comprising SEQ ID NO: 4, as shown in FIG. 9A. Any of the multimeric proteins comprising one or more sets of interface variants (e.g., CH1:CL electrostatic variant, CH1:CL steric variant, VH:VL electrostatic variant) can also have one or more amino acid substitutions (or one or more sets of amino acid substitutions) in a CH2 domain and/or CH3 domain of a Fc domain (e.g., a first variant Fc domain, a second variant Fc domain), including but not limited to: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below. Any of the multimeric proteins can also have one or more amino acid substitutions (or one or more sets of amino acid substitutions) described herein in the Fc domains set forth in the Figures including but not limited to, FIGS. 13A-13F, 14, 15, 16, and 17 or in the heterodimeric Fc monomers set forth in the Figures including but not limited to FIGS. 18A-18D, 19A-19C, 20A-20C, 21A-21C, and 22A-22C.

In another exemplary embodiment, the multimeric protein comprises: (a) a first monomer comprising SEQ ID NO: 5; (b) a second monomer comprising SEQ ID NO: 6; (c) a third monomer comprising SEQ ID NO: 7; and (d) a fourth monomer comprising SEQ ID NO: 8, as shown in FIG. 9B. Any of the multimeric proteins comprising one or more sets of interface variants (e.g., CH1:CL electrostatic variant, CH1:CL steric variant, VH:VL electrostatic variant) can also have one or more amino acid substitutions (or one or more sets of amino acid substitutions) in a CH2 domain and/or CH3 domain of a Fc domain (e.g., a first variant Fc domain, a second variant Fc domain), including but not limited to: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below. Any of the multimeric proteins can also have one or more amino acid substitutions (or one or more sets of amino acid substitutions) described herein in the Fc domains set forth in the Figures including but not limited to, FIGS. 13A-13F, 14, 15, 16, and 17 or in the heterodimeric Fc monomers set forth in the Figures including but not limited to FIGS. 18A-18D, 19A-19C, 20A-20C, 21A-21C, and 22A-22C.

In another exemplary embodiment, the multimeric protein comprises: (a) a first monomer comprising SEQ ID NO: 9; (b) a second monomer comprising SEQ ID NO: 10; (c) a third monomer comprising SEQ ID NO: 11; and (d) a fourth monomer comprising SEQ ID NO: 12, as shown in FIG. 9C. Any of the multimeric proteins comprising one or more sets of interface variants (e.g., CH1:CL electrostatic variant, CH1:CL steric variant, VH:VL electrostatic variant) can also have one or more amino acid substitutions (or one or more sets of amino acid substitutions) in a CH2 domain and/or CH3 domain of a Fc domain (e.g., a first variant Fc domain, a second variant Fc domain), including but not limited to: (i) Fc ADCC variants, (ii) v90 variants, (iii) Fc variants that increase binding to FcγRIIIa/CD16A, (iv) Fc variants that promote heterodimerization, (v) Fc variants that increase binding to FcRn (“FcRn variants”), (vi) skew variants, (vii) pI variants, (viii) Fc ablation variants, or any other Fc variant(s) described herein, as well as any combination(s) thereof. Further, any of the multimeric proteins can also independently and optionally be produced in a cell line that eliminates or reduces the incorporation of fucose into the glycosylation of the multimeric protein, as described in further detail below. Any of the multimeric proteins can also have one or more amino acid substitutions (or one or more sets of amino acid substitutions) described herein in the Fc domains set forth in the Figures including but not limited to, FIGS. 13A-13F, 14, 15, 16, and 17 or in the heterodimeric Fc monomers set forth in the Figures including but not limited to FIGS. 18A-18D, 19A-19C, 20A-20C, 21A-21C, and 22A-22C.

In another exemplary embodiment, the multimeric protein comprises: (a) a first monomer comprising a VH-CH1, wherein VH is a variable heavy domain; and (b) a second monomer comprising a VL-CL domain, wherein VL is a variable light domain, and wherein: (1) the variable heavy domain and the variable light domain form an antigen binding domain that binds to a target antigen of interest; (2) the first monomer and the second monomer comprise a set of VH:VL electrostatic variants comprising amino acid substitutions (i) Q39E:Q38K, or (ii) Q39K:Q38E; (3) the first monomer and the second monomer comprise a set of CH1:CL electrostatic variants comprising amino acid substitutions K213E/K218D:D122K/E123K; (4) the first monomer and the second monomer comprise a set of CH1:CL steric variants comprising amino acid substitutions A141F:F118A; (5) numbering of the set of VH:VL electrostatic variants, numbering of the set of CH1:CL electrostatic variants, and numbering of the set of CH1:CL steric variants are each according to EU numbering; and (6) the first monomer binds to the second monomer by CH1-CL dimerization and VH-VL association.

In another exemplary embodiment, the multimeric protein comprises: (a) a first monomer comprising a VH-CH1, wherein VH is a variable heavy domain; and (b) a second monomer comprising a VL-CL domain, wherein VL is a variable light domain, and wherein: (1) the variable heavy domain and the variable light domain form an antigen binding domain that binds to a target antigen of interest; (2) the first monomer and the second monomer comprise a set of VH:VL electrostatic variants comprising amino acid substitutions (i) Q39E:Q38K, or (ii) Q39K:Q38E; (3) the first monomer and the second monomer comprise a set of CH1:CL electrostatic variants comprising amino acid substitutions K213E/K218D:D122K/E123K; (4) the first monomer and the second monomer comprise a set of CH1:CL steric variants comprising amino acid substitutions A141F:F116A; (5) numbering of the set of VH:VL electrostatic variants, numbering of the set of CH1:CL electrostatic variants, and numbering of the set of CH1:CL steric variants are each according to EU numbering; and (6) the first monomer binds to the second monomer by CH1-CL dimerization and VH-VL association.

In another exemplary embodiment, the multimeric protein comprises: (a) a first monomer comprising a VH-CH1, wherein VH is a variable heavy domain; and (b) a second monomer comprising a VL-CL domain, wherein VL is a variable light domain, and wherein: (1) the variable heavy domain and the variable light domain form an antigen binding domain that binds to a target antigen of interest; (2) the first monomer and the second monomer comprise a set of VH:VL electrostatic variants comprising amino acid substitutions (i) Q39E:Q38K, or (ii) Q39K:Q38E; (3) the first monomer and the second monomer comprise a set of CH1:CL electrostatic variants comprising amino acid substitutions K213E/K218D:D122K/E123K; (4) the first monomer and the second monomer comprise a set of CH1:CL steric variants comprising amino acid substitutions K147S:S131K; (5) numbering of the set of VH:VL electrostatic variants, numbering of the set of CH1:CL electrostatic variants, and numbering of the set of CH1:CL steric variants are each according to EU numbering; and (6) the first monomer binds to the second monomer by CH1-CL dimerization and VH-VL association.

### V. IgG Fc Variants

As described in further detail above, the present invention encompasses multimeric proteins that contain useful variant IgG Fc domains. Such variant IgG Fc domains include amino acid modifications (i.e., substitutions, insertions, or deletions) to enhance FcTR-mediated cytotoxicity, increase serum half-life, and facilitate the self-assembly and/or purification of the multimers.

### A. Fc Variants for Increasing Antibody-Dependent Cellular Cytotoxicity (ADCC)

There are a number of useful Fc substitutions that can be made to alter binding to one or more of the FcγR receptors. Substitutions that result in increased binding (or in some cases, decreased binding) can be useful. For example, it is known that increased binding to FcγRIIIa can result in increased ADCC (antibody dependent cell-mediated cytotoxicity). In some instances, decreased binding to FcγRIIb (an inhibitory receptor) can be beneficial as well. Amino acid substitutions that find use in the present invention include those listed in U.S. Ser. No. 11/124,620 (particularly FIG. 41), U.S. Ser. No. 11/174,287, U.S. Ser. No. 11/396,495, U.S. Ser. No. 11/538,406, all of which are expressly incorporated herein by reference in their entirety and specifically for the variants disclosed therein.

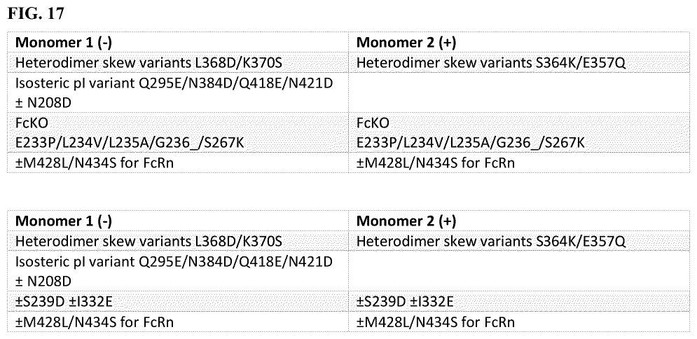

In some embodiments, provided herein are multimeric proteins containing Fc variants that increase antibody-dependent cellular cytotoxicity (ADCC; the cell-mediated reaction wherein nonspecific cytotoxic cells that express FcTRs recognize bound antibody on a target cell and subsequently cause lysis of the target cell) activity of the multimeric proteins. In other words, the multimeric proteins encompassed by the disclosure herein can include amino acid substitutions in each or both of the Fc monomeric domains of a parental sequence, usually IgG1, that can enhance ADCC. Exemplary Fc ADCC variants are shown in FIGS. 16 and 17.

In some embodiments, the Fc ADCC variants (e.g., ADCC-enhanced Fc variants) comprise amino acid substitution(s) selected from the group including:S239D, S239E, I332D, I332E, S239D/I332E, S239E/I332E, S239D/I332D, S239E/I332D, S239D/A330L/I332E, S239D/A330L, A330L/I332E, F243L, F243L/R292P/Y300L/V305I/P396L, I332E/P247I/A339Q, S298A/E333A, S298A/E333A/K334A, V264I/I332E, S298A, S298A/I332E, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264II332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E298R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering. In some embodiments, the amino acid substitution(s) present in an Fc ADCC variants are selected from the group including: S239D, S239E, I332D, I332E, S239D/I332E, S239E/I332E, S239D/I332D, S239E/I332D, S239D/A330L/I332E, S239D/A330L, A330L/I332E, S239D/I332N, S239N/I332D, A330Y/I332E, A330L/I332E, L328V/I332E, L328T/I332E, S239Q/V264I/I332E, S239E/V264I/A330Y/I332E, L235D/S239D/A330Y/I332E, S239D/V240I/A330Y/I332E, S239D/V264T/A330Y/I332E, S239D/K326E/A330Y/I32E, S234D/K326T/A330Y/I332E, E274R, P227G, G236S, D221K, H224E, K246H, D249Y, R255Y, E258H, E258Y, T260H, E283H, E283L, V284E, S239D/3272II332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering.

In some embodiments, a first variant Fc domain and/or a second variant Fc domain of the multimeric proteins provided comprise an Fc ADCC variant selected from the group including: S239D, S239E, I332D, I332E, S239D/I332E, S239E/I332E, S239D/I332D, S239E/I332D, S239D/A330L/I332E, S239D/A330L, A330L/I332E, S239D/I332N, S239N/I332D, A330Y/I332E, A330L/I332E, L328V/I332E, L328T/I332E, S239Q/V264II332E, S239E/V264I/A330Y/I332E, L235D/S239D/A330Y/I332E, S239D/V240I/A330Y/I332E, S239D/V264T/A330Y/I332E, S239D/K326E/A330Y/I32E, S234D/K326T/A330Y/I332E, E274R, P227G, G236S, D221K, H224E, K246H, D249Y, R255Y, E258H, E258Y, T260H, E283H, E283L, V284E, S239D/3272II332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering.

In some embodiments, one or more of these variants can be included either in both of the first variant Fc domain and the second variant Fc domain or in only one of the Fc domains (i.e., either the first variant Fc domain of the VH1-CH1-hinge-CH2-CH3 monomer or the second variant Fc domain of the VH2-CH1-hinge-CH2-CH3 monomer) of a multimeric protein. In some embodiments, a multimeric protein described includes ADCC-enhanced variants which includes one or more amino acid modifications in a first variant Fc domain (of the VH1-CH1-hinge-CH2-CH3 monomer) and/or a second variant Fc domain (of the VH2-CH1-hinge-CH2-CH3 monomer), in other words, in the first variant Fc domain alone, in the second variant Fc domain alone, or in both the first and second variant Fc domains. In some instances, a first variant Fc domain includes an Fc ADCC variant (e.g., the variant Fc domain of the first monomer of the multimeric protein), and a second variant Fc domain (e.g., the variant Fc domain of the third monomer of the multimeric protein) does not include an Fc ADCC variant, resulting in an asymmetrical distribution of Fc ADCC variants. In other instances, a first variant Fc domain includes an Fc ADCC variant, and a second variant Fc domain includes an Fc ADCC variant. In one embodiment, the Fc ADCC variant of the first variant Fc domain and the second variant Fc domain can be the same amino acid substitution. In one embodiment, the Fc ADCC variant of the first variant Fc domain and the second variant Fc domain can be different amino acid substitutions.

In some embodiments, the Fc ADCC variants described bind with greater affinity to the FcγRIIIa (CD16A) human receptor. In some embodiments, the Fc variants have affinity for FcγRIIIa (CD16A) that is at least 1-fold, 5-fold, 10-fold, 100-fold, 200-fold, or 300-fold greater than that of the parental Fc domain.

In some embodiments, the Fc ADCC variants described can mediate effector function more effectively in the presence of effector cells. In some embodiments, the Fc variants mediate ADCC that is greater than that mediated by the parental Fc domain. In certain embodiments, the Fc variants mediate ADCC that is at least 5-fold, 10-fold, 20-fold, 30-fold, 40-fold, or 50-fold greater than that mediated by the parental Fc domain.

Additional detailed descriptions of Fc variants that may enhance ADCC are provided in WO2004/029207, which is expressly incorporated herein by reference in its entirety and specifically for the variants disclosed therein.

### 1. Fc v90 Variants:

In some embodiments, a multimeric protein includes one or more Fc domains with enhanced binding to human FcγRIIIa (CD16A) and thus increased ADCC activity (“an Fc ADCC variant”). In some embodiments, multimeric protein includes the amino acid substitutions S239D/I332E (sometimes referred to as the “v90 variants”) in the CH2 domain of one or both of the first variant Fc domain and the second variant Fc domain of the multimeric protein, according to EU numbering. In some embodiments, a multimeric protein described herein comprises the Fc v90 variants (e.g., amino acid substitutions S239D/I332E) in both the first variant Fc domain and the second variant Fc domain. In some embodiments, a multimeric protein described herein comprises the Fc v90 variants in only one of the variant Fc domains. In some embodiments, the multimeric protein comprises the Fc v90 variants in one of the variant Fc domains and lacks the Fc v90 variants in the other variant Fc domain (e.g., in either the first variant Fc domain of the first monomer of the multimeric protein or the second variant Fc domain of the third monomer of the multimeric protein). In some embodiments, the multimeric protein comprises the Fc v90 variants in a first variant Fc domain and an amino acid substitution S239D in the CH2 domain of a second variant Fc domain, according to EU numbering. In certain embodiments, the multimeric protein comprises the Fc v90 variants in a first variant Fc domain and an amino acid substitution I332E in the CH2 domain of the second variant Fc domain, according to EU numbering. In some embodiments, the multimeric protein comprises the Fc v90 variants in a first variant Fc domain and lacks an amino acid substitution selected from S239D, I332E and S239D/I332E in the CH2 domain of the second variant Fc domain, according to EU numbering. In some embodiments, a first variant Fc domain comprises the S239D variant and a second variant Fc domain comprises the I332E variant. In some embodiments, a first variant Fc domain comprises the S239D variant and a second variant Fc domain comprises no Fc ADCC variant. In some embodiments, a first variant Fc domain comprises the I332E variant and a second variant Fc domain comprises no Fc ADCC variant.

As will be appreciated by those in the art, in the case of these asymmetrical Fc ADCC variants, which variant Fc domain (i.e., the first variant Fc domain of the first monomer of the multimeric protein or the second variant Fc domain of the third monomer of the multimeric protein) receives which variant(s) can be based on the “strandedness” outlined herein; that is, it may be useful to calculate the pI of different combinations and utilize the Fc ADCC variants such that the pIs of the two monomers are different to facilitate purification.

In some embodiments, monomer 1 (e.g., the first variant Fc domain) comprises a first Fc v90 variants, and monomer 2 (e.g., the second variant Fc domain) comprises the amino acid substitution S239D or 1332E. In some embodiments, monomer 1 comprises the Fc V90 variants, and monomer 2 does not comprise the amino acid substitution(s) S239D, 1332E or S239D/I332E. In some embodiments, at least one of the monomers of the multimeric protein comprises the Fc v90 variants (e.g., the first monomer of the multimeric protein, the third monomer of the multimeric protein). A first monomer may comprise the Fc v90 variants, or it may comprise a parental sequence relative to the Fc v90 variants (e.g., a wild-type Fc domain, a Fc domain with one or more amino acid modifications that improves ADCC but does not include S239D, 1332E or S239D/I332E substitutions, and the like). In such instances where at least one of the monomers comprises a parental sequence, relative to the Fc v90 variants, for the purposes of this section, this monomer may be referred to as a “WT Fc domain” with respect to the S239 and 1332 positions of the Fc domain. In some embodiments, the multimeric protein described herein comprises a first monomer having an amino acid substitution of either S239D, I332E, or S239D/I332E (such as, for example, the first variant Fc domain of the first monomer of the multimeric protein), and a second monomer having an amino acid substitution of either S239D, I332E, or S239D/I332E (such as, for example, the second variant Fc domain of the third monomer of the multimeric protein). In some embodiments, the multimeric protein described herein comprises a first monomer having an amino acid substitution of either S239D, I332E, or S239D/I332E, and a second monomer without an amino acid substitution of either S239D, I332E, or S239D/I332E.

In some embodiments, a first monomer (e.g., the first variant Fc domain) and a second monomer (e.g., the second variant Fc domain) contain a set of ADCC-enhanced variant substitutions (first monomer variant:second monomer variant) selected from the group including:S239: I332E; S239D:S239D; S239D:WT; S239D:S239D/I332E; S239D/I332E: WT; S239D/I332E:S239D; S239D/I332E:I332E; S239D/I332E:S239D/I332E; I332E:WT; I332E:I332E; I332E:S239D; I332E:S239D/I332E; WT:S239D; WT:I332E; WT: S239D/I332E, according to EU numbering. In some embodiments, monomer 1 and monomer 2 contain a set of ADCC-enhanced variant substitutions (monomer 1: monomer 2) selected from the group including:S239: I332E; S239D:S239D; S239D:WT; S239D:S239D/I332E; S239D/I332E:WT; S239D/I332E:S239D; S239D/I332E:I332E; S239D/I332E: S239D/I332E; I332E:WT; I332E:I332E; I332E:S239D; I332E:S239D/I332E; WT:S239D; WT:I332E; WT:S239D/I332E, according to EU numbering.

In some embodiments, monomers with enhanced ADCC can further comprise one or more additional modifications at one or more of the following positions, including, but not limited to, 236, 243, 298, 299, or 330 in the CH2 domain, according to EU numbering. In some embodiments, the first monomer comprises an amino acid substitution including, but not limited to: 236A, 243L, 298A, 299T, or 330L in the CH2 domain, according to EU numbering.

In some embodiments, an ADCC-enhanced Fc variant further includes, but is not limited, an amino acid substitution at one or more positions of the CH2 domain, according to EU numbering selected from the group including: position 236, 243, 298, 299, and 330. In some embodiments, an ADCC-enhanced Fc variant includes an amino acid substitution selected from the group including: 236A, 243L, 298A, 299T, 330L, 239D/332E, 236A/332E, 239D/332E/330L, 332E/330L, and any combination thereof in the CH2 domain, according to EU numbering. In some embodiments, a first monomer (i.e., the first variant Fc domain) and/or a second monomer (i.e., the second variant Fc domain) comprises an ADCC-enhanced Fc variant including, but not limited to, an amino acid substitution selected from the group including: 236A, 243L, 298A, 299T, 330L, 239D/332E, 236A/332E, 239D/332E/330L, 332E/330L, and any combination thereof in the CH2 domain, according to EU numbering, such that the Fc ADCC variant is the same in both monomers. Alternatively, the Fc ADCC variant is a different variant in each of the monomers.

Engineered multimeric proteins comprising such ADCC-enhanced Fc variants can also have higher-affinity FcγRIIIa binding, thus resulting in stronger ADCC activity with cells, such as but not limited to, NK cells. Multimeric proteins having a variant Fc domain described herein can be useful and effective for immune cell-mediated killing of tumor cells.

### 2. Fc Variants to Increase Binding to FcγRIIIa/CD16A:

There are additional Fc substitutions that find use in enhancing FcγRIIIa binding. In some embodiments, select monomers of the multimeric proteins (e.g., the first variant Fc domain of the first monomer of the multimeric protein, the second variant Fc domain of the third monomer of the multimeric protein) provided include one or more domains or monomers having increased binding to FcγRIIIa as compared to human IgG1 produced in standard research and production cell lines. In some embodiments, the Fc variants with improved binding affinity to at least FcγRIIIa have amino acid substitution(s) selected from the group including: V264I/I332E, S298A, S298A/I332E, S298A/E333A/K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E298R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering of the Fc domain. Additional Fc variants with enhanced binding affinity, specificity and/or avidity to FcγRIIIa are disclosed the specification and FIG. 41 of U.S. Pat. No. 8,188,231.

The described multimeric proteins contain such Fc variants that provide enhanced effector function and substantial increases in affinity for FcγRIIIa. In some embodiments, the Fc variants improve binding to FcγRIIIa allotypes such as, for example, both V158 and F158 polymorphic forms of FcγRIIIa. The FcTR binding affinities of these Fc variants can be evaluated using assay recognized by those skilled in the art including, but not limited to, a Surface Plasmon Resonance (SPR) and/or a BLI binding assay (such as Biacore, Octet, or Carterra LSA).

### B. Fc Variants for Increasing Binding to FcRn

Provided herein are additional Fc substitutions that find use in increased binding to the FcRn receptor and increased serum half-life, as specifically disclosed in U.S. Ser. No. 12/341,769, hereby incorporated by reference in its entirety, including, but not limited to, N434S, N434A, M428L, V308F, V259I, M428L/N434S, M428L/N434A, V259I/V308F, Y436I/M428L, Y436I/N434S, Y436V/N434S, Y436V/M428L, M252Y/S254T/T256E, and V2591/V308F/M428L. Such modification may be included in one or more domains or monomers of the subject multimeric proteins (such as, for example, the first variant Fc domain, the second variant Fc domain).

In some embodiments, additional Fc variants can increase serum half-life of a multimeric protein compared to a parental Fc domain. In some embodiments, the Fc variants have one or more amino acid modifications (i.e., substitutions, insertions or deletions) at one or more of the following amino acid residues or positions selected from the group including: 234, 235, 238, 250, 252, 254, 256, 265, 272, 286, 303, 305, 307, 311, 312, 317, 322, 340, 356, 360, 362, 376, 378, 380, 382, 413, 424, 428, and 434, according to EU numbering of the Fc region.

In some embodiments, the Fc variants have one or more amino acid substitutions selected from the group including: 234F, 235Q, 250E, 250Q, 252T, 252Y, 254T, 256E, 428L, 428F, 434S, 434A, 428L/434S, 428L/434A, 252Y/254T/256E, 234F/235Q/252T/254T/256E/322Q, 250E/428F, 250E/428L, 250Q/428F, and 250Q/428L, according to EU numbering.

In some embodiments, multimeric proteins described can include M428L/N434S or M428L/N434A substitutions in one or both variant Fc domains (i.e., the first variant Fc domain and/or the second variant Fc domain), which can result in longer half-life in serum. In more embodiments, a first monomer (e.g., the first variant Fc domain of the first monomer of the multimeric protein) or a second monomer (e.g., the second variant Fc domain of the third monomer of the multimeric protein) include M428L/N434S substitutions. In more embodiments, a first monomer (e.g., the first variant Fc domain) and a second monomer (e.g., the second variant Fc domain) include M428L/N434S substitutions. In certain embodiments a first monomer (e.g., the first variant Fc domain) or a second monomer (e.g., the second variant Fc domain) include M428L/N434A substitutions. In certain embodiments, a first monomer (e.g., the first variant Fc domain) and a second monomer (e.g., the second variant Fc domain) include M428L/N434A substitutions. In various embodiments a first monomer (e.g., the first variant Fc domain) or a second monomer (e.g., the second variant Fc domain) include M252Y/S254T/T256E substitutions. In certain embodiments, a first monomer (e.g., the first variant Fc domain) and a second monomer (e.g., the second variant Fc domain) include M252Y/S254T/T256E substitutions. Such substitutions can result in longer half-life in serum of molecules comprising such.

### C. Fc Variants for Heterodimerization

In some embodiments, the multimeric proteins provided herein include two variant Fc domain sequences (i.e., a first variant Fc domain within the first monomer and a second variant Fc domain within the third monomer of the subject multimeric protein). Such variant Fc domains include amino acid modifications to facilitate the self-assembly and/or purification of the proteins.

An ongoing problem in antibody technologies is the desire for “bispecific” proteins that bind to two different antigens simultaneously, in general thus allowing the different antigens to be brought into proximity and resulting in new functionalities and new therapies. In general, these multimeric proteins are made by including genes for each heavy and light chain into the host cells. This generally results in the proper formation of the desired Fc heterodimer (A-B), as well as the two Fc homodimers (A-A and B-B (not including the light chain heterodimeric issues also described herein)). However, a major obstacle in the formation of bispecific, multimeric proteins is the difficulty in biasing the formation of the desired Fc heterodimers over the formation of the Fc homodimers and/or purifying the Fc heterodimers away from the homodimers.

There are a number of mechanisms that can be used to generate the subject multimeric proteins. In addition, as will be appreciated by those in the art, these different mechanisms can be combined to ensure high heterodimerization of the Fc domains. Amino acid modifications that facilitate the production and purification of heterodimeric Fc domains are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (also referred to as “knobs-into-holes” or “KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric Fc domains away from homodimers are further discussed in detailed below.

There are a number of suitable pairs of sets of heterodimerization skew variants. These variants come in “pairs” of “sets.” That is, one set of the pair is incorporated into a first monomer (e.g., the first variant Fc domain of the first monomer of the multimeric protein) and the other set of the pair is incorporated into a second monomer (e.g., the second variant Fc domain of the third monomer of the multimeric protein). It should be noted that these sets do not necessarily behave as “knobs in holes” variants, with a one-to-one correspondence between a residue on one monomer and a residue on the other; that is, these pairs of sets form an interface between the two monomers that encourages heterodimer formation (of the Fc domains) and discourages homodimer formation (of the Fc domains), allowing the percentage of heterodimers that spontaneously form under biological conditions to be over 90%, rather than the expected 50% (25% homodimer A/A:50% heterodimer A/B:25% homodimer B/B).

### 1. Skew Variants:

In some embodiments, the multimeric protein includes skew (e.g., steric) variants which are one or more amino acid modifications in a first monomer (A; e.g., the first variant Fc domain) and/or a second monomer (B; e.g., the second variant Fc domain) that favor the formation of Fc heterodimers (Fc dimers that include the first and second variant Fc domains; (A-B) over Fc homodimers (Fc dimers that include two of the first variant Fc domain or two of the second variant Fc domain; A-A or B-B). Suitable skew variants are included in the FIG. 29 of US2016/0355608, hereby incorporated by reference in its entirety and specifically for its disclosure of skew variants, as well as in FIGS. 13 and 17.

Thus, suitable Fc heterodimerization variant pairs that will permit the formation of heterodimeric Fc regions are shown in the figures including FIGS. 13 and 17. Thus, a first monomer (e.g., the first variant Fc domain of the first monomer of the multimeric protein) has first Fc heterodimerization variants and the second monomer (e.g., the second variant Fc domain of the third monomer of the multimeric protein) has second Fc heterodimerization variants selected from the pairs in FIGS. 13 and 17.

One mechanism is generally referred to in the art as “knobs and holes,” referring to amino acid engineering that creates steric influences to favor heterodimeric formation and disfavor homodimeric formation (with respect to Fc domains) can also optionally be used; this is sometimes referred to as “knobs and holes,” as described in U.S. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety. The Figures identify a number of “monomer A-monomer B” pairs (generally, first variant Fc domain-second variant Fc domain) that rely on “knobs and holes.” In addition, as described in Merchant et al., Nature Biotech. 16:677 (1998), these “knobs and hole” mutations can be combined with disulfide bonds to skew formation to heterodimerization.

An additional mechanism that finds use in the generation of heterodimeric Fc domains is sometimes referred to as “electrostatic steering” as described in Gunasekaran et al., J. Biol. Chem. 285(25):19637 (2010), hereby incorporated by reference in its entirety. This mechanism is also sometimes referred to herein as “charge pairs.” In this embodiment, electrostatics are used to skew the formation towards heterodimerization (of the first and second variant Fc domains). As those in the art will appreciate, these variants may also have an effect on pI, and thus on purification, and thus could in some cases also be considered pI variants. However, as these were generated to force heterodimerization and were not used as purification tools, they are classified as “steric variants.” These include, but are not limited to, D221E/P228E/L368E paired with D221R/P228R/K409R (e.g., these are “monomer corresponding sets”) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the multimeric protein includes one or more sets of such heterodimerization skew variants. These variants come in “pairs” of “sets.” That is, one set of the pair is incorporated into a first monomer (e.g., the first variant Fc domain) and the other set of the pair is incorporated into a second monomer (e.g., the second variant Fc domain). It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation (of the variant Fc domains) and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization skew variants are depicted in FIGS. 13 and 17. Such skew variants include, but are not limited to:S364K/E357Q:L368D/K370S; L368D/K370S:S364K; L368E/K370S:S364K; T411E/K360E/Q362E:D401K; L368D/K370S:S364K/E357L; K370S:S364K/E357Q (EU numbering). In terms of nomenclature, the pair “S364K/E357Q:L368D/K370S” means that one of the monomers has the double variant set S364K/E357Q and the other has the double variant set L368D/K370S.

In exemplary embodiments, the multimeric protein includes Fc heterodimerization variants as sets:S364K/E357Q:L368D/K370S; L368D/K370S:S364K; L368E/K370S:S364K; T411E/K360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C or T366S/L368A/Y407V/S354C:T366W/Y349C) are all skew variant amino acid substitution sets of Fc heterodimerization variants. In an exemplary embodiment, the multimeric protein includes a “S364K/E357Q:L368D/K370S” amino acid substitution set. In terms of nomenclature, the pair “S364K/E357Q:L368D/K370S” means that one of the monomers includes an Fc domain (e.g., the first variant Fc domain) that includes the amino acid substitutions S364K and E357Q and the other monomer (e.g., the second variant fc domain) includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US 2012/0149876, herein incorporated by reference, particularly for its disclosure of skew variants), pI variants, isotypic variants, FcRn variants, ablation variants, etc. into one or both of the first and second monomers of the multimeric protein. Further, individual modifications can also independently and optionally be included or excluded from the subject the heterodimeric antibody.

Additional monomer A and monomer B variants that can be combined with other variants, optionally and independently in any amount, such as pI variants outlined herein or other steric variants that are shown in FIG. 37 of US 2012/0149876, the figure and legend and SEQ ID NOs of which are incorporated expressly by reference herein.

In some embodiments, the steric variants outlined herein can be optionally and independently incorporated with any pI variant (or other variants such as, for example, Fc ADCC variants, FcRn variants, etc.) into one or both monomers (i.e., into one or both of the first and second variant Fc domains), and can be independently and optionally included or excluded from the monomers of the multimeric proteins described herein.

A subset of skew variants is “knobs in holes” (KIH) variants. Exemplary “knob-in-hole” variants are depicted in FIG. 7 of U.S. Pat. No. 8,216,805, which is incorporated herein by reference. Such “knob-in-hole” variants include, but are not limited to: an amino acid substitution at position 347, 349, 350, 351, 357, 366, 368, 370, 392, 394, 395, 397, 398, 399, 405, 407 and/or 409 of the CH3 constant domain of an IgG such as an IgG1, IgG2a, IgG2b, or IgG4 (Kabat numbering). In some embodiments, the “knob-in-hole “variants include, but are not limited to: an amino acid substitution at Y349, L351, E357, T366, L368, K370, N390, K392, T394, D399, S400, F405, Y407, K409, R409, T411, or any combination thereof of the CH3 domain of an IgG such as an IgG1, IgG2a, IgG2b, IgG4 (EU numbering). In some embodiments, the “knob-in-hole” variants include, but are not limited to: one or more amino acid substitutions including Y349D/E, L351D/K/Y, E357K, T366A/K/Y, L368E, K370E, N390D/K/R, K392E/F/L/M/R, T394W, D399K/R/W/Y, S400D/E/K/R, F405A/I/M/S/T/V/W, Y407A/Y, K409E/D/F, R409E/D/F, and T411D/E/K/N/Q/R/W.

In some embodiments, such variants include one or more amino acid substitutions including, but not limited to: Y349C, E357K, S354C, T366S, T366W, T366Y, L368A, K370E, T394S T394W, D399K, F405A, F405W, Y407A, Y407T, Y407V, R409D, T366Y/F405A, T394W/Y407T, T366W/F405W, T394S/Y407A, F405W/Y407A, and T366W/T394S (EU numbering). In some embodiments, these variants include knob:hole paired substitutions including, but not limited to: T366W:Y407V; S354C/T366W:Y349C/T366S/Y407V; Y349C/T366W:S354C/T366S/L368A/Y407V; Y349C/T366W/R409D/K370E:S354C/T366S/L368A/Y407V/D399K/E357K; R409D/K370E:D399K/E357K; T366W:T366S/L368A/Y407V; T366W/R409D/K370E:T366S/L368A/Y407V/D399K/E357K; T366W:T366S/L368A/Y407V; T366W/Y366Y:T366S/L368A/T394W/F405A/Y407V; Y349C/T366W:S354C/T366S/L368A/Y407V; Y349C/T366W/R409D/K370E:S354C/T366S/L368A/Y407V/D399K/E357K paired substitutions, according to EU numbering.

Additional exemplary “knob-into-hole” variants as described by the amino acid substitutions of the CH3 domains can be found in, for example, Carter et al., J. Immunol. Methods, 248(1-2):7-15 (2001), Merchant et al. Nat. Biotechnol. 16(7):677-81 (1998), Ridgway et al. Protein Eng. 9(7):617-2 (1996), and U.S. Pat. Nos. 8,216,805 and 10,287,352, the disclosures of which are herein incorporated by reference in their entireties.

### 2 pI (Isoelectric Point) Variants for Fc Heterodimers:

In some embodiments, the multimeric protein includes purification variants that advantageously allow for the separation of heterodimeric Fc domains from homodimeric proteins.

There are several basic mechanisms that can lead to ease of purifying multimeric proteins comprising heterodimeric Fc domains. For example, modifications to one or both of the multimeric protein heavy chain monomers A and B (i.e., modifications to one or both of the first variant Fc domain and/or the second variant Fc domain of the multimeric protein) such that each monomer comprising a variant Fc domain has a different pI allows for the isoelectric purification of heterodimeric Fc domain pairing (“A-B”) from monomeric (“A-A” and “B-B”) proteins. As described above, it is also possible to “skew” the formation of heterodimers over homodimers (with respect to Fc domains) using skew variants. Thus, a combination of heterodimerization skew variants and pI variants find particular use in the multimeric proteins provided herein.

Additionally, as more fully outlined below, depending on the format of the multimeric proteins, pI variants either contained within the constant region and/or Fc domains of a monomer, and/or domain linkers can be used. In some embodiments, the multimeric protein includes additional modifications for alternative functionalities that can also create pI changes, such as Fc, FcRn and KO variants.

In some embodiments, the subject multimeric proteins provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the multimeric protein, pI variants can be either contained within the constant and/or Fc domains of a monomer, or charged linkers, either domain linkers or scFv linkers, can be used. That is, antibody formats that utilize scFv(s) can include charged scFv linkers (either positive or negative), that give a further pI boost for purification purposes. As will be appreciated by those in the art, some formats are useful with just charged scFv linkers and no additional pI adjustments, although the multimeric proteins described herein do provide pI variants that are on one or both of the variant Fc domains (i.e., the first variant Fc domain and/or the second variant Fc domain), and/or charged domain linkers as well. In addition, additional amino acid engineering for alternative functionalities may also confer pI changes, such as Fc, FcRn and KO variants.

In subject multimeric proteins for which pI is used as a separation mechanism to allow the purification of heterodimeric Fc domains, amino acid variants are introduced into one or both of the variant Fc domains (i.e., in the first variant Fc domain of the first monomer of the multimeric protein and/or the second variant Fc domain of the third monomer of the multimeric protein). That is, the pI of one of the variant Fc domains (referred to herein for simplicity as “monomer A”) can be engineered away from monomer B (referring to the other variant Fc domain), or both monomer A and B can be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (e.g., aspartic acid to lysine) or changing a charged residue to a neutral residue (e.g., loss of a charge; lysine to serine). A number of these pI variants are shown in the FIGS. 13, 14, and 17.

Thus, in some embodiments, the subject multimeric protein includes amino acid modifications in the constant regions that alter the isoelectric point (pI) of at least one, if not both, of the variant Fc domains of the multimeric proteins to form “pI multimeric proteins”) by incorporating amino acid substitutions (“pI variants” or “pI substitutions”) into one or both of the variant Fc domains. As shown herein, the separation of the Fc heterodimers from the two Fc homodimers can be accomplished if the pIs of the two monomers differ by as little as 0.1 pH unit, with 0.2, 0.3, 0.4 and 0.5 or greater all finding use in the multimeric proteins described herein.

As will be appreciated by those in the art, the number of pI variants to be included on each or both variant Fc domain(s) to achieve good separation will depend in part on the starting pI of the components. That is, to determine which monomer to engineer or in which “direction” (e.g., more positive, or more negative), the Fv sequences of the two target antigens are calculated and a decision is made from there. As is known in the art, different Fvs will have different starting pIs which are exploited in the multimeric proteins described herein. In general, as outlined herein, the pIs are engineered to result in a total pI difference of each monomer of at least about 0.1 logs, with 0.2 to 0.5 being preferred as outlined herein.

In the case where pI variants are used to achieve heterodimerization of the variant Fc domains, by using the constant region(s) of the heavy chain(s), a more modular approach to designing and purifying bispecific multimeric proteins, including antibodies, is provided. Thus, in some embodiments, heterodimerization variants (including skew and pI heterodimerization variants) are not included in the variable regions, such that each individual multimeric protein must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively, or in addition to isotypic substitutions, the possibility of immunogenicity resulting from the pI variants is significantly reduced by utilizing isosteric substitutions (e.g., Asn to Asp; and Gln to Glu).

As discussed below, a side benefit that can occur with this pI engineering is also the extension of serum half-life and increased FcRn binding. That is, as described in US2012/0028304 (incorporated by reference in its entirety), lowering the pI of multimeric protein constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific multimeric proteins, as the ability to either eliminate, minimize, and distinguish when Fc homodimers are present is significant. Similarly, the ability to reliably test the reproducibility of the multimeric protein production is important.

In general, embodiments of particular use rely on sets of variants that include skew variants, which encourage Fc heterodimerization formation over Fc homodimerization formation, coupled with pI variants, which increase the pI difference between the two variant Fc domains to facilitate purification of Fc heterodimers away from Fc homodimers.

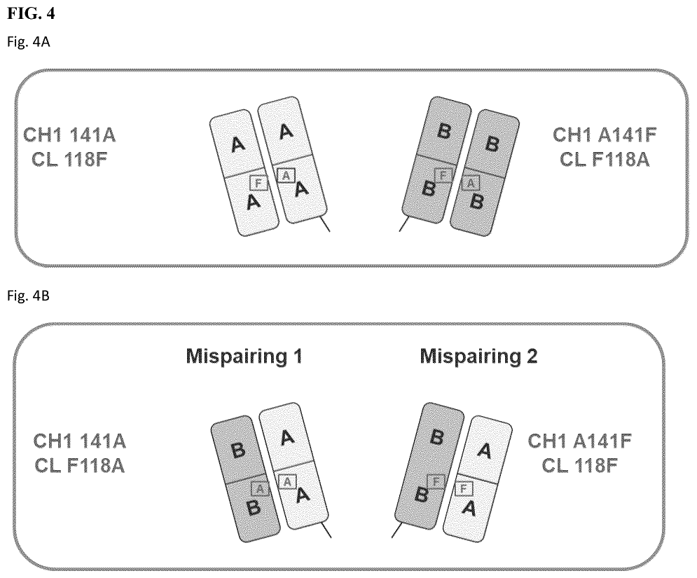

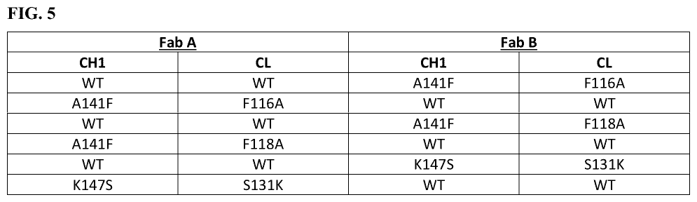

Exemplary combinations of pI variants are shown in FIGS. 4 and 5, and FIG. 30 of US2016/0355608, all of which are herein incorporated by reference in its entirety and specifically for the disclosure of pI variants. Preferred combinations of pI variants are shown in FIGS. 13, 14, and 17. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one variant Fc domain (the negative Fab side) comprising 208D/295E/384D/418E/421D variants (N208D/Q295E/N384D/Q418E/N421D when relative to human IgG1) and the other variant Fc domain (the positive scFv side) comprising a positively charged scFv linker, including (GKPGS)4 (SEQ ID NO: 194). However, as will be appreciated by those in the art, the first and third monomer of the multimeric protein includes a CH1 domain, including position 208. Accordingly, in constructs that do not include a CH1 domain (for example for fusion proteins that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 13 and the other monomer has a charged linker (either in the form of a charged scFv linker because that monomer comprises an scFv or a charged domain linker, as the format dictates, which can be selected from those depicted in FIG. 26).

In some embodiments, modifications are made in the hinge of the variant Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389, 392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

### 3. Isotypic Variants:

In addition, many embodiments of the multimeric proteins described herein rely on the “importation” of pI amino acids at particular positions from one IgG isotype into another, thus reducing or eliminating the possibility of unwanted immunogenicity being introduced into the variants. A number of these are shown in FIG. 21 of US 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significantly affect the pI of the multimeric protein. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the multimeric protein can be seen. As discussed herein, having the pIs of the two monomers differ by at least 0.5 can allow separation by ion exchange chromatography or isoelectric focusing, or other methods sensitive to isoelectric point. 4. Calculating p:

The pI of each monomer can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US 2014/0370013. As discussed herein, which monomer to engineer is generally decided by the inherent pI of the Fv and scaffold regions. Alternatively, the pI of each monomer can be compared.

5. pI Variants that also Confer Better FcRn In Vivo Binding:

In the case where the pI variant decreases the pI of the monomer, they can have the added benefit of improving serum retention in vivo.

Although still under examination, Fc regions are believed to have longer half-lives in vivo, because binding to FcRn at pH 6 in an endosome sequesters the Fc (Ghetie and Ward, 1997, Immunol Today, 18(12): 592-598, entirely incorporated by reference). The endosomal compartment then recycles the Fc to the cell surface. Once the compartment opens to the extracellular space, the higher pH 7.4, induces the release of Fc back into the blood. In mice, Dall'Acqua et al. showed that Fc mutants with increased FcRn binding at pH 6 and pH 7.4 actually had reduced serum concentrations and the same half-life as wild-type Fc (Dall'Acqua et al., 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

It has been suggested that antibodies with variable regions that have lower isoelectric points may also have longer serum half-lives (Igawa et al., 2010, PEDS, 23(5): 385-392, entirely incorporated by reference). However, the mechanism of this is still poorly understood. Constant region variants with reduced pI and extended half-life would provide a more modular approach to improving the pharmacokinetic properties of multimeric proteins, as described herein.

### 6. Additional Fc Variants for Additional Functionality:

In addition to the Fc heterodimerization variants discussed above, there are a number of useful Fc amino acid modification that can be made for a variety of reasons, including, but not limited to, altering binding to one or more FcTR receptors, altered binding to FcRn receptors, etc., as discussed herein.

Accordingly, the multimeric proteins provided herein can include such amino acid modifications with or without the heterodimerization variants outlined herein (e.g., the pI variants and steric variants). Each set of variants can be independently and optionally included or excluded from any particular multimeric protein.

### 7. Additional Heterodirnerization Variants:

In some embodiments, the first variant Fc domain (of the first monomer of the multimeric protein) comprises one or more amino acid substitutions selected from the group including: L351Y, D399R, D399K, S400D, S400E, S400R, S400K, F405A, F4051, F405M, F405T, F405S, F405V, F405W, Y407A, Y407I, Y407L, Y407V, and any combination thereof, and the second variant Fc domain (of the third monomer of the multimeric protein) comprises one or more amino acid substitutions selected from the group including: T350V, T366A, T366I, T366L, T366M, T366Y, T366S, T366C, T366V, T366W, N390D, N390E, N390R, K392L, K392M, K3921, K392D, K392E, T394W, K409F, K409W, T411N, T411R, T411Q, T411K, T411D, T411E, T411W, and any combination thereof.

In some embodiments, other heterodimerization pair variants include, but are not limited to, amino acid substitutions of L234A/L235A:wildtype; L234A/L235A:L234K/L235K; L234D/L235E:L234K/L235K; E233A/L234D/L235E:E233A/L234R/L235R; L234D/L235E: E233K/L234R/L235R; E233A/L234K/L235A:E233K/L234A/L235K; E269Q/D270N: E269K/D270R; and WT:L235K/A327K of the CH2 domain, according to the EU numbering.

In some embodiments, the first and/or second variant Fc domains comprise one or more amino acid substitutions selected from the group including:S239D, D265S, S267D, E269K, S298A, K326E, A330L and 1332E. In certain instances, the Fc paired variants include, but are not limited to, S239D/D265S/I332E/E269K:S239D/D265S/S298A; S239D/K326E/A330L/I332E:S298A or S239D/K326E/A330L/I332E/E269K:S298A of the CH2 domain, according to EU numbering.

Additional descriptions of useful heterodimeric variants are disclosed in U.S. Pat. Nos. 9,732,155; 10,457,742 and 10,875,931 and US2021/0277150 and US2020/0087414, the disclosures of which, including the description of Fc domain variants are herein incorporated by reference in their entireties.

### D. Ablation Variants

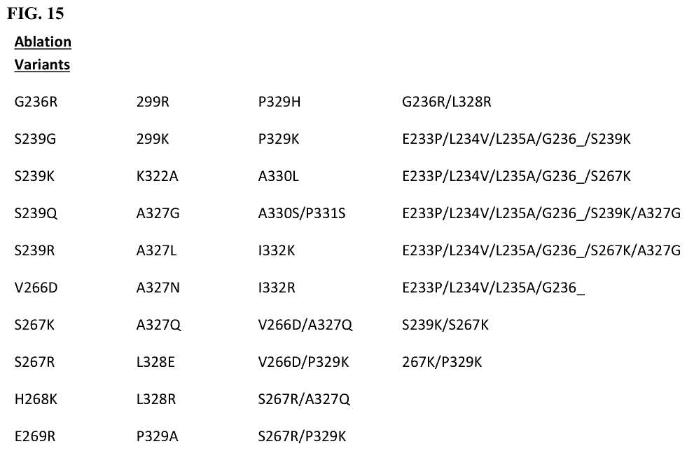

While, in general, multimeric proteins that engage natural killer (NK) cells retain at binding to CD16A (including “wild type” binding or increased binding to CD16A as outlined above), in some cases, surprisingly, NK engager activity can be seen even when binding to CD16A has been reduced or ablated. Accordingly, provided is another category of functional Fc variants to include are “FcγR ablation variants” or “Fc knock out (FcKO or KO)” variants. In these embodiments, it is desirable to reduce or remove the normal binding of a variant Fc domain to one or more or all of the Fc7 receptors (e.g., FcTR1, FcγRIIa, FcγRIIb, FcγRIIIa, etc.) to avoid additional mechanisms of action. That is, for example, in many embodiments, particularly in the use of bispecific, multimeric proteins that bind a target antigen monovalently, it is generally desirable to ablate FcγRIIIa binding to eliminate or significantly reduce ADCC activity, wherein one of the variant Fc domains (e.g., the first variant Fc domain or the second variant Fc domain) comprises one or more Fc receptor ablation variants. These ablation variants are depicted in FIGS. 15 and 17, and each can be independently and optionally included or excluded, with preferred aspects utilizing ablation variants selected from the group including G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcTR binding but generally not FcRn binding.

As is known in the art, the Fc domain of human IgG1 has the highest binding to the Fc7 receptors, and thus ablation variants can be used when the constant domain (or variant Fc domain) in the backbone of the multimeric protein is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fc7 receptors, and thus those backbones can be used with or without the ablation variants.

### E. Combination of Heterodimeric and Fc Variants

As will be appreciated by those in the art, all of the recited heterodimerization variants (including skew and/or pI variants) can be optionally and independently combined in any way, as long as they retain their “strandedness” or “monomer partition.” In addition, all of these variants can be combined into any of the heterodimerization formats.

In the case of pI variants, while embodiments finding particular use are shown in the Figures, other combinations can be generated, following the basic rule of altering the pI difference between two monomers to facilitate purification.

In addition, any of the heterodimerization variants, skew, and pI, are also independently and optionally combined with Fc ADCC variants, Fc variants, FcRn variants, or Fc ablation variants, as generally outlined herein.

Exemplary combination of variants that are included in some embodiments of the multimeric proteins are included in FIG. 17. Alternatively, the FcRn variants can be M428L/N434A or M252Y/S243T/T256E.

Accordingly, the multimeric proteins provided herein can include such amino acid modifications with or without the heterodimerization variants outlined herein (e.g., the pI variants and steric variants). Each set of variants can be independently and optionally included or excluded from any particular heterodimeric protein.

### F. Afucosylated Fc Domains

In some embodiments, the increased binding of a Fc domain to CD16A is the result of producing the multimeric protein in a cell line that reduces or eliminates the incorporation of fucose into the glycosylation of the multimeric protein. See, for example, Pereira et al., MAbs (2018) 10(5):693-711.

In some embodiments, multimeric proteins comprising variant Fc domains described are produced in a host cell such that the variant Fc domains have reduced fucosylation or no fucosylation compared to a parental Fc domain. In some instances, multimeric proteins described are produced in a genetically modified host cell, wherein the genetic modification to the host cell results in the overexpression of 0(1,4)-N-acetylglucosaminyltransferase III (GnTIII), a glycosyltransferase catalyzing the formation of bisected oligosaccharides, which are generally also non-fucosylated. N-glycosylation of the (variant) Fc domain can play a role in binding to FcγR; and afucosylation of the N-glycan can increase the binding capacity of the (variant) Fc domain to FcγRIIIa. As discussed in further detail above, an increase in FcγRIIIa binding can enhance ADCC, which can be advantageous in certain antibody therapeutic applications in which cytotoxicity is desirable.

In some embodiments, a variant Fc domain (e.g., the first variant domain, the second variant domain) is engineered such that it has reduced fucosylation or no fucosylation, compared to a parental Fc domain. In the context of an Fc domain, the terms “afucosylation,” “afucosylated,” “defucosylation,” and “defucosylated” are used interchangeably, and generally refer to the absence or removal of core-fucose from the N-glycan attached to the CH2 domain of an Fc domain. For instance, an afucosylated antibody lacks core fucosylation in the Fc domain. As used herein, the phrase “a low level of fucosylation” or “reduced fucosylation” generally refers to an overall fucosylation level in a specific (variant) Fc domain that is no more than about 10.0%, no more than 5.0%, no more than 2.5%, no more than 1.0%, no more than about 0.5%, no more than 0.25%, no more than about 0.1%, or no more than 0.01%, compared to the fucosylation level of parental Fc domain. The term “% fucosylation” generally refers to the level of fucosylation in a specific (variant) Fc domain compared to that of a parental Fc domain. The % fucosylation can be measured according to any suitable method known in the relevant art, such as, for example, by mass spectrometry (MS), HPLC-Chip Cube MS (Agilent), and reverse phase-HPLC.

In some embodiments, a particular level of fucosylation is desired. In some embodiments, a variant Fc domain is provided, wherein the variant Fc domain comprises a particular level of afucosylation. In some further embodiments, the fucosylation level of the variant Fc domain is no more than about 10.0%, no more than about 9.0%, no more than about 8.0%, no more than about 7.0%, no more than about 6.0%, no more than about 5.0%, no more than about 4.0%, no more than about 3.0%, no more than about 2.0%, no more than about 1.5%, no more than about 1.0%, no more than about 0.5%, no more than 0.25%, no more than about 0.1%, or no more than 0.01%, compared to that of a parental Fc domain.

In some embodiments, multimeric proteins comprising afucosylated Fc domain(s) can be enriched (to obtain a particular level of afucosylation) by affinity chromatography using resins conjugated with a fucose binding moiety, such as, for example, an antibody or lectin specific for fucose, with some embodiments finding particular utility when fucose is present in a 1-6 linkage (see, e.g., Kobayashi et al., 2012, J. Biol. Chem. 287:33973-82).

In some embodiments, the fucosylated species of the variant Fc domain(s) can be separated from the afucosylated species of the Fc domain (to obtain a particular level of afucosylation) using an anti-fucose specific antibody in an affinity column. Alternatively, or in addition to, afucosylated species can be separated from fucosylated species based on the differential binding affinity to FcγRIIIa using affinity chromatography (again, to obtain a particular level of afucosylation).

### VI. Nucleic Acids

The disclosure further provides nucleic acid compositions encoding the multimeric proteins provided herein, including, but not limited to, tetrameric and dimeric proteins.

As will be appreciated by those in the art, the nucleic acid compositions will depend on the format and scaffold of the multimeric protein. Thus, for example, when the format requires four amino acid sequences, such as for the subject tetrameric proteins (wherein the multimeric protein comprises a first monomer comprising a VH1-CH1-hinge-CH2-CH3 monomer, wherein VH1 is a first variable heavy domain and CH2-CH3 is a first variant IgG Fc domain; a second monomer comprising a VL1-CL1 monomer, wherein VL1 is a first variable light domain, and wherein the first variable heavy domain and the first variable light domain form a first antigen binding domain; a third monomer comprising a VH2-CH1-hinge-CH2-CH3 monomer, wherein VH2 is a second variable heavy domain and CH2-CH3 is a second variant IgG Fc domain; and a fourth monomer comprising a VL2-CL2 monomer, wherein VL2 is a second variable light domain, and wherein the second variable heavy domain and the second variable light domain form a second antigen binding domain), four nucleic acid sequences can be incorporated into one or more expression vectors for expression. Similarly, some formats (e.g., dual scFv formats such as disclosed in FIG. FIG. 2 of U.S. Pat. No. 10,793,632) only two nucleic acids are needed; again, they can be put into one or two expression vectors.

As is known in the art, the nucleic acids encoding the components of the multimeric proteins described herein can be incorporated into expression vectors as is known in the art, and depending on the host cells used to produce the multimeric proteins described herein. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the multimeric proteins described herein are then transformed into any number of different types of host cells as is well known in the art, including mammalian, bacterial, yeast, insect and/or fungal cells, with mammalian cells (e.g., CHO cells), finding use in many embodiments.

In some embodiments, nucleic acids encoding each monomer are each contained within a single expression vector, generally under control of different or the same promoter(s). In embodiments of particular use in the multimeric proteins described herein, each of the monomers encoded by nucleic acids are contained on a different expression vector. As shown herein and in U.S. Pat. No. 9,822,186, hereby incorporated by reference, different vector ratios can be used to drive heterodimer formation.

The multimeric proteins described herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI, and the Fc heterodimer also has a distinct pI, thus facilitating isoelectric purification of the multimeric proteins (e.g., anionic exchange columns, cationic exchange columns). These substitutions also aid in the determination and monitoring of any contaminating multimeric proteins with mispaired heavy chains.

### VII. Biological and Biochemical Functionality of the Multimeric Proteins

Generally, the multimeric proteins described herein are administered to patients with a disease, disorder, or condition (e.g., cancer), and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

VIII. Treatments

Once made, the compositions of the invention find use in a number of applications. In some instances, the compositions of the invention find use in a number of oncology applications, by treating cancer, generally by enhancing immune responses, including, promoting T cell activation, activating NK cells, enhancing NK cell mediated lysis of tumor cells and providing co-stimulation to T cells in the tumor environment. Such compositions can be combined with proinflammatory cytokines for increased cytotoxicity against cancer or tumor cells.

Alternatively, or in addition to, the compositions of the invention find use in a number of applications outside of oncology, such as, for example, the treatment of asthma, inflammatory diseases, autoimmune diseases, and infection.

### A. Multimeric Protein Compositions for In Vivo Administration

Formulations of the multimeric proteins used in accordance with the multimeric proteins described herein are prepared for storage by mixing a multimeric protein having the desired degree of purity with optional pharmaceutically acceptable carriers, excipients, or stabilizers (Remington's Pharmaceutical Sciences 16th edition, Osol, A. Ed. [1980]), in the form of lyophilized formulations or aqueous solutions.

### B. Administrative Modalities

The multimeric proteins provided herein administered to a subject, in accord with known methods, such as intravenous administration as a bolus or by continuous infusion over a period of time.

### C. Treatment Modalities

In the methods described herein, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MM) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the disclosure includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for a therapy may also be measured by its ability to stabilize the progression of disease. The ability of a compound to inhibit the disease may be evaluated in an animal model system.

### EXAMPLES

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### A. Example 1-Solving Heavy Chain/Light Chain Pairing Problem

A number of antibody formats have been explored for bispecific targeting. For example, antibody fragments of many different forms have been generated, including BiTE (Goebeler et al. (2016) Journal Clinical Oncology, 34(10): 1104-1111, herein incorporated by reference), DART (Root et al. (2016) Antibodies, 5(1), 6; Tsai et al. (2016) Molecular Therapy Oncolytics, 3, 15024, all of which are herein incorporated by reference), Nanobody (Bannas et al. (2017) Front Immunol, 8: 1603, herein incorporated by reference), and TandAb (Reusch et al. (2014) mAbs, 6:3, 727-738; Reusch et al. (2015) mAbs, 7:3, 584-604, all of which are herein incorporated by reference) formats which have advanced into clinical testing. However, these antibody fragments clear rapidly in vivo as they lack the constant region of intact IgG that provides binding to various Fc receptors and ligands that maintain long half-life in serum (i.e., the neonatal Fc receptor, FcRn). To address such shortcomings of fragment-based bispecifics, formats having Fc regions have been generated, such as the HFE BiTE format (Lorenczewski et al. (2017) Blood, 130:2815; PCT Publication No. WO 2017/134140, all of which are herein incorporated by reference) and the 1+1 Fab-scFv-Fc format (referred to as the Triple F format in PCT Publication No. WO 2014/110601, herein incorporated by reference). Nonetheless, these formats differ structurally from the natural IgG format, and may fall short of the evolutionarily optimized pharmacokinetic properties and immunogenic profile inherent to the natural IgG format.

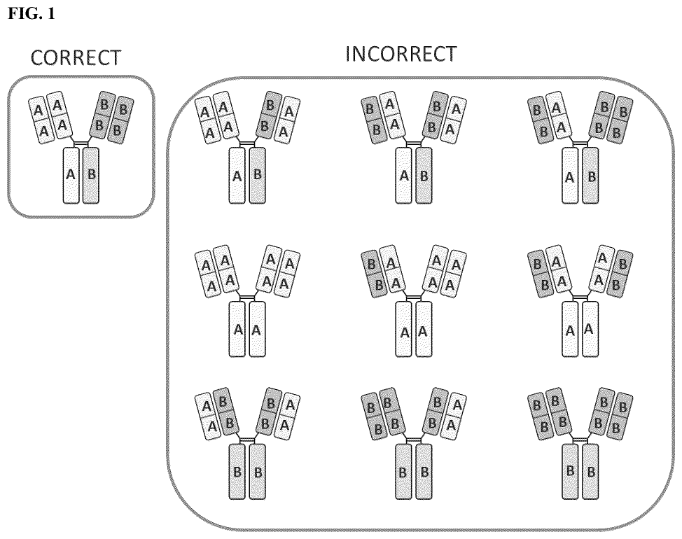

However, production of bispecific antibodies in IgG format is challenging, as antibody heavy chains bind antibody light chains in a relatively promiscuous manner. As a result of this promiscuous pairing, concomitant expression of, e.g., two antibody heavy chains and two antibody light chains naturally leads to heavy chain homodimerization and/or scrambling of heavy chain/light chain pairings. This potentially results in one correct bispecific pairing in a mixture with nine incorrect pairings, as depicted in FIG. 1.

There are numerous approaches to circumvent the problem of heavy chain homodimerization, including those described in U.S. Pat. No. 10,858,417. However, circumventing the scrambling of heavy chain/light chain pairing has been more difficult due to the complex multidomain heterodimeric interactions within antibody Fabs (i.e., assembly driven by both VH/VL and CH1/CL domain interactions).

(A)(1). Engineering electrostatic variants in the CH1:CL interface:

A first strategy is based on identification of a salt bridge formed by K213/K218 in the CH1 and D122/E123 in the CL (positions in EU numbering). Without being limited by theory, substitutions are engineered at these residues to skew towards formation of correct heavy chain/light chain pairs by an electrostatic steering mechanism. As depicted in FIG. 2A, the goal is to engineer substitutions in the CH1 and/or CL so that Heavy Chain A pairs with Light Chain A and Heavy Chain B pairs with Light Chain B by virtue of electrostatic complementarity. Conversely, as depicted in FIG. 2B, the substitutions disfavor the pairing of Heavy Chain A with Light Chain B and Heavy Chain B with Light Chain A. While FIG. 2 depicts K213E/K218D:D122K/E123K in CH1:CL interface of Fab A and WT in Fab B, additional substitution pairings as depicted in FIG. 3 are also contemplated.

(A)(2). Engineering steric variants in the CH-1:CL interface:

A second strategy for developing variants described herein is based on a “knob-in-hole” pair formed by A141 in the CH1 and F116 in the CL; A141F in the CH1 and F118 in the CL; and K147 in CH1 and S131 in the CL (positions in EU numbering). Without being limited by theory, substitutions are engineered at these residues to skew towards formation of correct heavy chain/light chain pairs by a steric hindrance mechanism. As depicted in FIG. 4A, the goal is to engineer substitution in the CH1 and/or CL so that Heavy Chain A pairs with Light Chain A and Heavy Chain B pairs with Light Chain B by virtue of steric complementarity. Conversely, as depicted in FIG. 4B, the substitutions disfavor the pairing of Heavy Chain A with Light Chain B and Heavy Chain B with Light Chain A. While FIG. 4 depicts WT in Fab A and A141F:F118A in CH1:CL interface of Fab B, additional substitution pairings as depicted in FIG. 5 are also contemplated.

(A)(3). Engineering electrostatic variants in the V-L:VL interface:

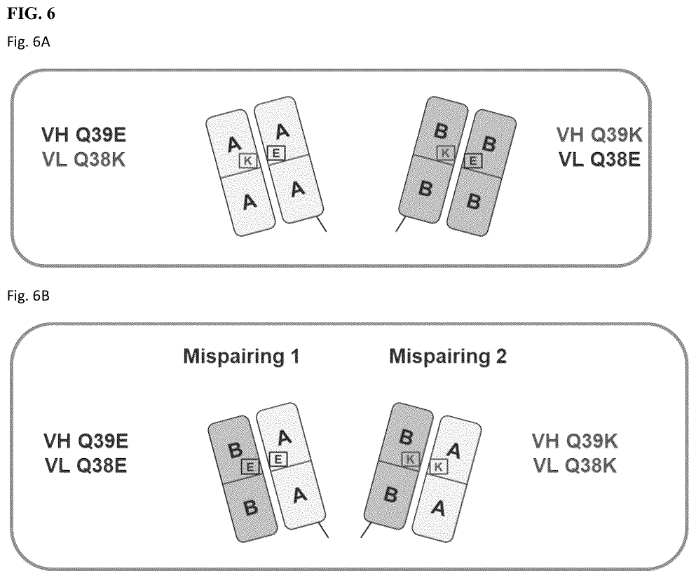

A third strategy is based on a hydrogen bonded pair formed by Q39 in the VH and Q38 in the VL (positions in Kabat numbering). By electrostatic steering mechanism, substitutions engineered at these residues to skew towards formation of correct heavy chain/light chain pairs. As depicted in FIG. 6A, the goal is to engineer substitutions in the VH and/or VL so that Heavy Chain A pairs with Light Chain A and Heavy Chain B pairs with Light Chain B by virtue of electrostatic complementarity. Conversely, as depicted in FIG. 6B, the substitutions disfavor the pairing of Heavy Chain A with Light Chain B and Heavy Chain B with Light Chain A. While FIG. 6 depicts Q39E:Q38K in VH:VL interface of Fab A and Q39K:Q38E in VH:VL interface of Fab B, additional substitution pairings as depicted in FIG. 7 are also contemplated.

(A)(4). Combining variants in the VH/VL and CH1:CL interfaces:

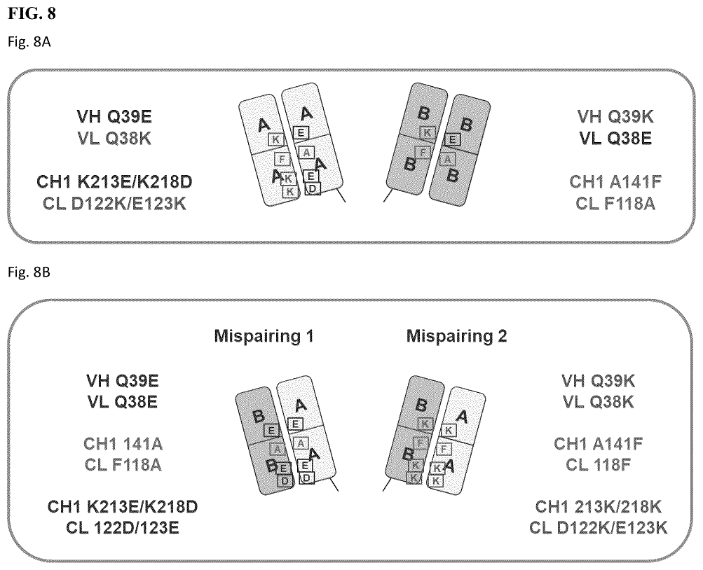

To fully leverage the multidomain interactions within the Fabs, another strategy is to combine each of the foregoing strategies, or to combine at least two of the foregoing strategies (such as, for example, VH:VL interface electrostatic variants and CH1:CL interface electrostatic variants, or VH:VL interface electrostatic variants and CH1:CL interface steric variants). As depicted in FIG. 8A, one such combination utilizes Q39E:Q38K in VH:VL interface and K213E/K218D:D122K/E123K in CH1:CL interface of Fab A and Q39K:Q38E in VH:VL interface and A141F:F118A in CH1:CL interface of Fab B; however, additional combinations of any of the substitutions in FIGS. 3, 5, and 7 are also contemplated. Sequences of illustrative bispecific IgGs which includes Q39E:Q38K in VH:VL interface and K213E/K218D:D122K/E123K in CH1:CL interface of Fab A and Q39K:Q38E in VH:VL interface and A141F:F116A, A141F:F118A, or K147S:S131K in the CH1:CL interface are depicted in FIG. 9.

### B. Example 2—Prodiction and Purification

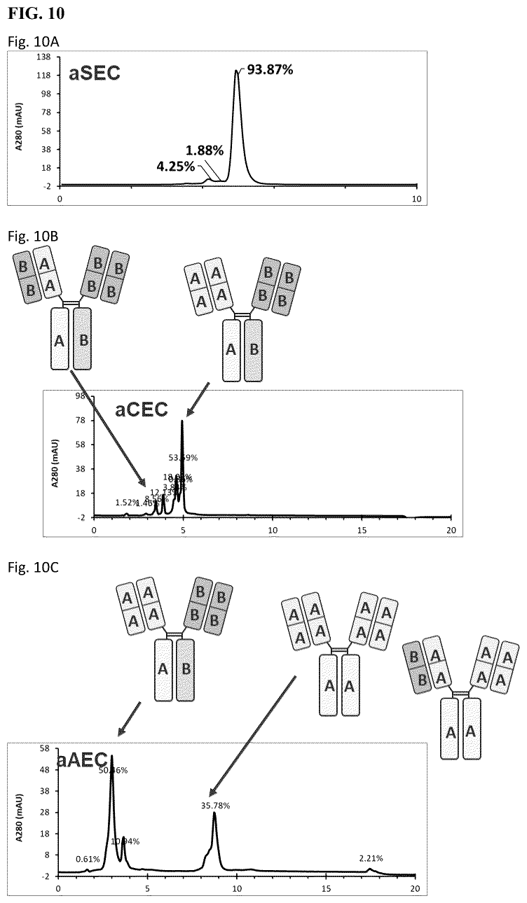

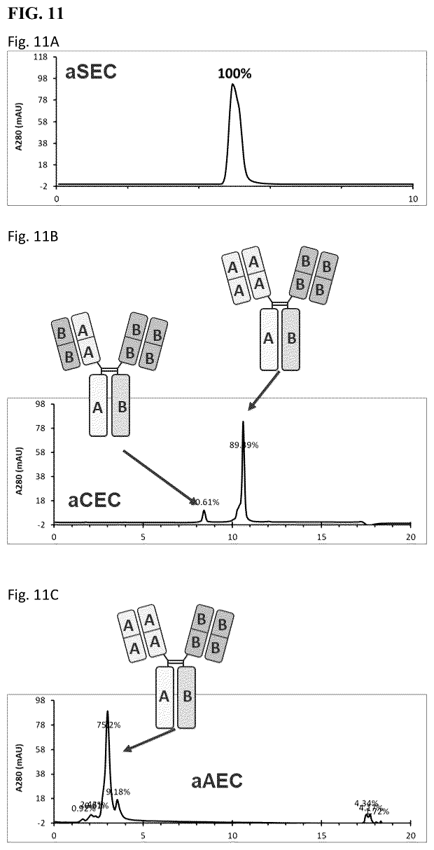

Even with a plethora of substitutions across both Fab arms, for example as described in Example 1D, to skew toward correct heavy chain/light chain pairing, it is difficult to completely abrogate formation of incorrect pairs. Using XENP40711 (sequences as depicted in FIG. 9) as an illustrative example, FIGS. 10A-C respectively depict separation of species using analytical size-exclusion chromatography (aSEC), analytical cation-exchange chromatography (aCEC), and analytical anion-exchange chromatography (aAEC) following Protein A purification. FIGS. 10B and 10C show that while correctly paired XENP40711 is the dominant species, there were in fact mispaired species as well.

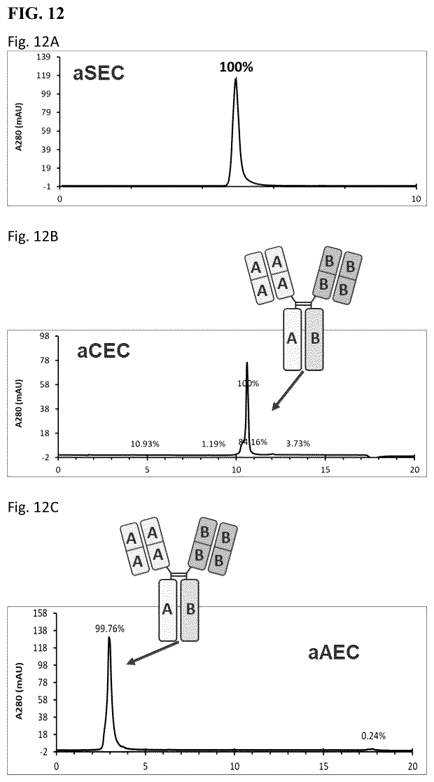

However, an additional advantage of the novel engineering as described herein and in Example 1A is that “Mispairing 1” as depicted in FIG. 2B results in a surface-exposed negatively charged patch which elutes earlier in cation exchange chromatographic purification; and conversely, “Mispairing 2” as depicted in FIG. 2B results in a surface-exposed positively charged patch which elutes later in anion exchange chromatographic purification. This enables a strategy for effectively removing mispaired species using CEC and AEC purification. FIGS. 11A-C respectively depicts separation of species using aSEC, aCEC, and aAEC following Protein A purification and AEC purification. As shown in FIG. 11C, negatively charged contaminating mispaired species were removed. FIGS. 12A-C respectively depicts separation of species using aSEC, aCEC, and aAEC following protein A purification, AEC purification, and CEC purification. As shown in FIGS. 12B-C, remaining positively charged contaminating mispaired species were also removed.

C. Example 3—Bispecifics incorporating Novel HC/LC Pairing Variants Demonstrate Good Pharimacokinetics

One of the motivations for engineering bispecific antibodies in the native IgG was to emulate its pharmacokinetic properties. Accordingly, cynomolgus monkeys were dosed with XENP40711, an exemplary bispecific incorporating novel HC/LC pairing variants. Serum concentration was determined by anti-human Fc capture and anti-human kappa detection. PK interpretative analysis was performed using Phoenix WinNonlin software (version 6.4.0.768) with PK parameters for non-compartmental analysis of free drug serum concentration versus time. Pharmacokinetic profile of XENP40711 is depicted in FIG. 26. Concentration-time profiles generally followed an apparent biphasic decline with estimates of the t_{1/2β} ranging from 7.24 to 11.1 days. The half-life of native IgG is roughly 10⁻²¹ days (depending on attributes such as the variable regions), so the data from this study suggests that the native IgG-like bispecific incorporating novel HC/CL pairing variants demonstrate good pharmacokinetic comparable to native IgG.

1. A multimeric protein, comprising:
(a) a first monomer comprising a VH1-CH1-hinge-CH2-CH3 monomer, wherein VH1 is a first variable heavy domain and CH2-CH3 is a first variant IgG Fc domain;
(b) a second monomer comprising a VL1-CL1 monomer, wherein VL1 is a first variable light domain, and wherein the first variable heavy domain and the first variable light domain form a first antigen binding domain;
(c) a third monomer comprising a VH2-CH1-hinge-CH2-CH3 monomer, wherein VH2 is a second variable heavy domain and CH2-CH3 is a second variant IgG Fc domain; and
(d) a fourth monomer comprising a VL2-CL2 monomer, wherein VL2 is a second variable light domain, and wherein the second variable heavy domain and the second variable light domain form a second antigen binding domain, wherein:
(1) the first monomer and the second monomer comprise a set of CH1:CL electrostatic variants and the third monomer and the fourth monomer comprise a set of CH1:CL steric variants, or the first monomer and the second monomer comprise a set of CH1:CL steric variants and the third monomer and the fourth monomer comprise a set of CH1:CL electrostatic variants,
(2) the set of CH1:CL electrostatic variants comprises a set of amino acid substitutions selected from the group consisting of: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R,
(3) the set of CH1:CL steric variants comprises a set of amino acid substitutions selected from the group consisting of: (i) A141F:F118A, (ii) A141F:F116A, and (iii) K147S:S131K,
(4) the first monomer and the second monomer comprise a first set of VH:VL electrostatic variants, and the third monomer and the fourth monomer comprise a second set of VH:VL electrostatic variants,
(5) the first set of VH:VL electrostatic variants and the second set of VH:VL electrostatic variants comprise amino acid substitutions selected from the group consisting of: (i) Q39E:Q38K and Q39K:Q38E, respectively, (ii) Q39K:Q38E and Q39E:Q38K, respectively, (iii) Q39E:Q38K and Q39R:Q38E, respectively, (iv) Q39E:Q38K and Q39R:Q38D, respectively, (v) Q39E:Q38K and Q39E:Q38K, respectively, (vi) Q39E:Q38K and Q39E:Q38R, respectively, (vii) Q39E:Q38K and Q39D:Q38K, respectively, (viii) Q39E:Q38K and Q39D:Q38R, respectively, (ix) Q39E:Q38R and Q39K:Q38E, respectively, (x) Q39E:Q38R and Q39K:Q38D, respectively, (xi) Q39E:Q38R and Q39R:Q38E, respectively, (xii) Q39E:Q38R and Q39R:Q38D, respectively, (xiii) Q39E:Q38R and Q39E:Q38K, respectively, (xiv) Q39E:Q38R and Q39E:Q38R, respectively, (xv) Q39E:Q38R and Q39D:Q38K, respectively, (xvi) Q39E:Q38R and Q39D:Q38R, respectively, (xvii) Q39D:Q38K and Q39K:Q38E, respectively, (xviii) Q39D:Q38K and Q39K:Q38D, respectively, (xix) Q39D:Q38K and Q39R:Q38E, respectively, (xx) Q39D:Q38K and Q39R:Q38D, respectively, (xxi) Q39D:Q38K and Q39E:Q38K, respectively, (xxii) Q39D:Q38K and Q39E:Q38R, respectively, (xxiii) Q39D:Q38K and Q39D:Q38K, respectively, (xxiv) Q39D:Q38K and Q39D:Q38R, respectively, (xxv) Q39D:Q38R and Q39K:Q38E, respectively, (xxvi) Q39D:Q38R and Q39K:Q38D, respectively, (xxvii) Q39D:Q38R and Q39R:Q38E, respectively, (xxviii) Q39D:Q38R and Q39R:Q38D, respectively, (xxix) Q39D:Q38R and Q39E:Q38K, respectively, (xxx) Q39D:Q38R and Q39E:Q38R, respectively, (xxxi) Q39D:Q38R and Q39D:Q38K, respectively, (xxxii) Q39D:Q38R and Q39D:Q38R, respectively, (xxxiii) Q39K:Q38E and Q39K:Q38E, respectively, (xxxiv) Q39K:Q38E and Q39K:Q38D, respectively, (xxxv) Q39K:Q38E and Q39R:Q38E, respectively, (xxxvi) Q39K:Q38E and Q39R:Q38D, respectively, (xxxvii) Q39E:Q38K and Q39K:Q38D, respectively, (xxxviii) Q39K:Q38E and Q39E:Q38R, respectively, (xxxix) Q39K:Q38E and Q39D:Q38K, respectively, (xl) Q39K:Q38E and Q39D:Q38R, respectively, (xli) Q39R:Q38E and Q39K:Q38E, respectively, (xlii) Q39R:Q38E and Q39K:Q38D, respectively, (xliii) Q39R:Q38E and Q39R:Q38E, respectively, (xliv) Q39R:Q38E and Q39R:Q38D, respectively, (xlv) Q39R:Q38E and Q39E:Q38K, respectively, (xlvi) Q39R:Q38E and Q39E:Q38R, respectively, (xlvii) Q39R:Q38E and Q39D:Q38K, respectively, (xlviii) Q39R:Q38E and Q39D:Q38R, respectively, (xlix) Q39K:Q38D and Q39K:Q38E, respectively, (1) Q39K:Q38D and Q39K:Q38D, respectively, (li) Q39K:Q38D and Q39R:Q38E, respectively, (lii) Q39K:Q38D and Q39R:Q38D, respectively, (liii) Q39K:Q38D and Q39E:Q38K, respectively, (liv) Q39K:Q38D and Q39E:Q38R, respectively, (lv) Q39K:Q38D and Q39D:Q38K, respectively, (lvi) Q39K:Q38D and Q39D:Q38R, respectively, (lvii) Q39R:Q38D and Q39K:Q38E, respectively, (lviii) Q39R:Q38D and Q39K:Q38D, respectively, (lix) Q39R:Q38D and Q39R:Q38E, respectively, (lx) Q39R:Q38D and Q39R:Q38D, respectively, (lxi) Q39R:Q38D and Q39E:Q38K, respectively, (lxii) Q39R:Q38D and Q39E:Q38R, respectively, (lxiii) Q39R:Q38D and Q39D:Q38K, respectively, and (lxiv) Q39R:Q38D and Q39D:Q38R, respectively, and
(6) numbering of the set of CH1:CL electrostatic variants, numbering of the set of CH1:CL steric variants, numbering of the first set of VH:VL electrostatic variants, and numbering of the second set of VH:VL electrostatic variants are all according to EU numbering.

2. The multimeric protein according to claim 1, wherein the set of CH1:CL electrostatic variants comprises amino acid substitutions K213E/K218D:D122K/E123K.

3. The multimeric protein according to claim 1, wherein the set of CH1:CL steric variants comprises amino acid substitutions A141F:F118A.

4. The multimeric protein according to claim 1, wherein:
(i) the first set of VH:VL electrostatic variants comprises amino acid substitutions Q39E:Q38K, and the second set of VH:VL electrostatic variants comprises amino acid substitutions Q39K:Q38E, or
(ii) the first set of VH:VL electrostatic variants comprises amino acid substitutions Q39K:Q38E, and the second set of VH:VL electrostatic variants comprises amino acid substitutions Q39E:Q38K.

5. (canceled)

6. The multimeric protein according to claim 1, wherein the first and/or second variant IgG Fc domains comprise one or more FcγRIIIA (CD16a) binding variant substitutions.

7. The multimeric protein according to claim 6, wherein the one or more FcγRIIIA (CD16a) binding variant substitutions are selected from the group consisting of: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

8. The multimeric protein according to claim 7, wherein the first and second variant IgG Fc domains comprise a set of FcγRIIIA (CD16a) binding variant substitutions selected from the group consisting of: (i) S239D/I332E:S239D/I332E, (ii) S239D S239D, (iii) 1332E:1332E, (iv) WT:S239D/I332E, (v) WT:S239D, (vi) WT:1332E, (vii) S239D/I332E:WT, (viii) S239D:WT, (ix) 1332E:WT, (x) S239D/I332E:S239D, (xi) S239D/I332E:1332E, (xii) S239D:S239D/I332E, (xiii) 1332E:S239D/I332E, (xiv) S239D:1332E, and (xv) 1332E:S239D, wherein numbering is according to EU numbering.

9. (canceled)

10. The multimeric protein according to claim 6, wherein the first and second variant IgG Fc domains comprise a set of heterodimerization variants selected from the group consisting of those depicted in FIGS. 13A-13F, wherein numbering is according to EU numbering.

11. The multimeric protein according to claim 10, wherein the set of heterodimerization variants is selected from the group consisting of: (i) S364K/E357Q: L368D/K370S, (ii) S364K:L368D/K370S, (iii) S364K:L368E/K370S, (iv) D401K:T411E/K360E/Q362E, and (v) T366W:T366S/L368A/Y407V, wherein numbering is according to EU numbering.

12. The multimeric protein according to claim 1, wherein the first and second variant IgG Fc domains further comprise;_(i) one or more ablation variants, and/or (ii) one or more pI variants.

13. The multimeric protein according to claim 12, wherein the one or more ablation variants are E233P/L234V/L235A/G236del/S267K, wherein numbering is according to EU numbering.

14. (canceled)

15. The multimeric protein according to claim 12, wherein the one or more pI variants are N208D/Q295E/N384D/Q418E/N421D, wherein numbering is according to EU numbering.

16. The multimeric protein according to claim 1,
wherein the first variant IgG Fc domain comprises amino acid variants S364K/E357Q/E233P/L234V/L235A/G236del/S267K, wherein the second variant IgG Fc domain comprises amino acid variants L368D/K370S/N208D/Q295E/N384D/Q418E/N421D/E233P/L234V/L235A/G236del/S267K, and wherein numbering is according to EU numbering.

17. The multimeric protein according to claim 1, wherein the first and second variant IgG Fc domains each further comprise amino acid variants M428L/N434S or M428L/N434A, wherein numbering is according to EU numbering.

18. The multimeric protein according to claim 1, wherein:
(A) the first variant IgG Fc domain is selected from the group consisting of: (i) a first variant IgG1 Fc domain, (ii) a first variant IgG2 Fc domain, and (iii) a first variant IgG4 Fc domain, and
(B) the second variant IgG Fc domain is selected from the group consisting of: (i) a second variant IgG1 Fc domain, (ii) a second variant IgG2 Fc domain, and (iii) a second variant IgG4 Fc domain.

19. (canceled)

20. A nucleic acid composition comprising nucleic acids encoding the first, second, third, and fourth monomers according to claim 1.

21. An expression vector comprising the nucleic acids according to claim 20.

22. A host cell transformed with an expression vector according to claim 21.

23. A method of making a multimeric protein, comprising:
(a) culturing the host cell according to claim 22 under conditions wherein the multimeric protein is expressed; and
(b) recovering the multimeric protein.

24. A multimeric protein, comprising:
(a) a first monomer comprising a VH1-CH1-hinge-CH2-CH3 monomer, wherein VH1 is a first variable heavy domain and CH2-CH3 is a first variant IgG Fc domain;
(b) a second monomer comprising a VL1-CL1 monomer, wherein VL1 is a first variable light domain, and wherein the first variable heavy domain and the first variable light domain form a first antigen binding domain;
(c) a third monomer comprising a VH2-CH1-hinge-CH2-CH3 monomer, wherein VH2 is a second variable heavy domain and CH2-CH3 is a second variant IgG Fc domain; and
(d) a fourth monomer comprising a VL2-CL2 monomer, wherein VL2 is a second variable light domain, and wherein the second variable heavy domain and the second variable light domain form a second antigen binding domain,
wherein either the first monomer and the second monomer or the third monomer and the fourth monomer comprise a set of CH1:CL electrostatic variants, wherein the set of CH1:CL electrostatic variants comprises amino acid substitutions at amino acid residues K213/K218:D122/E123, wherein numbering is according to EU numbering.

25.-46. (canceled)

47. A multimeric protein, comprising:
(a) a first monomer comprising a VH1-CH1-hinge-CH2-CH3 monomer, wherein VH1 is a first variable heavy domain and CH2-CH3 is a first variant IgG Fc domain;
(b) a second monomer comprising a VL1-CL1 monomer, wherein VL1 is a first variable light domain, and wherein the first variable heavy domain and the first variable light domain form a first antigen binding domain;
(c) a third monomer comprising a VH2-CH1-hinge-CH2-CH3 monomer, wherein VH2 is a second variable heavy domain and CH2-CH3 is a second variant IgG Fc domain; and
(d) a fourth monomer comprising a VL2-CL2 monomer, wherein VL2 is a second variable light domain, and wherein the second variable heavy domain and the second variable light domain form a second antigen binding domain,
wherein either the first monomer and the second monomer or the third monomer and the fourth monomer comprise a set of CH1:CL steric variants, wherein the set of CH1:CL steric variants comprises amino acid substitutions at amino acid residues selected from the group consisting of: (i) A141:F118, (ii) A141:F116, and (iii) K147:S131, wherein numbering is according to EU numbering.

48.-68. (canceled)

69. A multimeric protein, comprising:
(a) a first monomer comprising a VH1-CH1-hinge-CH2-CH3 monomer, wherein VH1 is a first variable heavy domain and CH2-CH3 is a first variant IgG Fc domain;
(b) a second monomer comprising a VL1-CL1 monomer, wherein VL1 is a first variable light domain, and wherein the first variable heavy domain and the first variable light domain form a first antigen binding domain;
(c) a third monomer comprising a VH2-CH1-hinge-CH2-CH3 monomer, wherein VH2 is a second variable heavy domain and CH2-CH3 is a second variant IgG Fc domain; and
(d) a fourth monomer comprising a VL2-CL2 monomer, wherein VL2 is a second variable light domain, and wherein the second variable heavy domain and the second variable light domain form a second antigen binding domain,
wherein (i) the first monomer and the second monomer comprise a first set of VH:VL electrostatic variants comprising amino acid substitutions at amino acid residues Q39:Q38, and (ii) the third monomer and the fourth monomer comprise a second set of VH:VL electrostatic variants, comprising amino acid substitutions at amino acid residues Q39:Q38, wherein numbering is according to Kabat numbering.

70.-90. (canceled)

91. A multimeric protein, comprising:
(a) a first monomer comprising a VH1-CH1-hinge-CH2-CH3 monomer, wherein VH1 is a first variable heavy domain and CH2-CH3 is a first variant IgG Fc domain;
(b) a second monomer comprising a VL1-CL1 monomer, wherein VL1 is a first variable light domain, and wherein the first variable heavy domain and the first variable light domain form a first antigen binding domain;
(c) a third monomer comprising a VH2-CH1-hinge-CH2-CH3 monomer, wherein VH2 is a second variable heavy domain and CH2-CH3 is a second variant IgG Fc domain; and
(d) a fourth monomer comprising a VL2-CL2 monomer, wherein VL2 is a second variable light domain, and wherein the second variable heavy domain and the second variable light domain form a second antigen binding domain,
wherein (i) the first monomer and the second monomer comprise a first set of VH:VL electrostatic variants and a set of CH1:CL electrostatic variants, and (ii) the third monomer and the fourth monomer comprise a second set of VH:VL electrostatic variants and a set of CH1:CL steric variants.

92.-114. (canceled)

115. A multimeric protein, comprising:
(a) a first monomer comprising a VH1-CH1-hinge-CH2-CH3 monomer, wherein VH1 is a first variable heavy domain and CH2-CH3 is a first variant IgG Fc domain;
(b) a second monomer comprising a VL1-CL1 monomer, wherein VL1 is a first variable light domain, and wherein the first variable heavy domain and the first variable light domain form a first antigen binding domain;
(c) a third monomer comprising a VH2-CH1-hinge-CH2-CH3 monomer, wherein VH2 is a second variable heavy domain and CH2-CH3 is a second variant IgG Fc domain; and
(d) a fourth monomer comprising a VL2-CL2 monomer, wherein VL2 is a second variable light domain, and wherein the second variable heavy domain and the second variable light domain form a second antigen binding domain,
wherein (i) the VH:VL interface of the first monomer and the second monomer comprises a first set of VH:VL electrostatic variants comprising amino acid substitutions at amino acid residues Q39:Q38, and the VH:VL interface of the third monomer and the fourth monomer comprises a second set of VH:VL electrostatic variants comprising amino acid substitutions at amino acid residues Q39:Q38, wherein numbering is according to Kabat numbering,
(ii) the CH1:CL interface of the first monomer and the second monomer and/or the CH1:CL interface of the third monomer and the fourth monomer comprises one or more amino acid substitutions, wherein the one or more amino acid substitutions comprises (1) a set of CH1:CL electrostatic variants, or (2) a set of CH1:CL steric variants, wherein numbering is according to EU numbering, and
(iii) the multimeric protein has antigen binding affinity substantially equivalent to a corresponding multimeric protein lacking the amino acid substitutions at the VH:VL interfaces and the CH1:CL interface(s).

116.-150. (canceled)

151. A multimeric protein, comprising:
(a) a first monomer comprising a VH1-CH1-hinge-CH2-CH3 monomer, wherein VH1 is a first variable heavy domain and CH2-CH3 is a first variant IgG Fc domain;
(b) a second monomer comprising a VL1-CL1 monomer, wherein VL1 is a first variable light domain, and wherein the first variable heavy domain and the first variable light domain form a first antigen binding domain;
(c) a third monomer comprising a VH2-CH1-hinge-CH2-CH3 monomer, wherein VH2 is a second variable heavy domain and CH2-CH3 is a second variant IgG Fc domain; and
(d) a fourth monomer comprising a VL2-CL2 monomer, wherein VL2 is a second variable light domain, and wherein the second variable heavy domain and the second variable light domain form a second antigen binding domain, wherein:
(i) the first monomer and the second monomer comprise a first set of VH:VL electrostatic variants comprising amino acid substitutions Q39E:Q38K, and a set of CH1:CL electrostatic variants comprising amino acid substitutions K213E/K218D:D122K/E123K; and
(ii) the third monomer and the fourth monomer comprise a second set of VH:VL electrostatic variants comprising amino acid substitutions Q39K:Q38E, and a set of CH1:CL steric variants comprising amino acid substitutions A141F:F116A; and
wherein numbering of the first set and second set of VH:VL electrostatic variants is according to Kabat numbering; and numbering of the set of CH1:CL electrostatic variants and the set of CH1:CL steric variants is according to EU numbering.

152. A multimeric protein, comprising:
(a) a first monomer comprising a VH1-CH1-hinge-CH2-CH3 monomer, wherein VH1 is a first variable heavy domain and CH2-CH3 is a first variant IgG Fc domain;
(b) a second monomer comprising a VL1-CL1 monomer, wherein VL1 is a first variable light domain, and wherein the first variable heavy domain and the first variable light domain form a first antigen binding domain;
(c) a third monomer comprising a VH2-CH1-hinge-CH2-CH3 monomer, wherein VH2 is a second variable heavy domain and CH2-CH3 is a second variant IgG Fc domain; and
(d) a fourth monomer comprising a VL2-CL2 monomer, wherein VL2 is a second variable light domain, and wherein the second variable heavy domain and the second variable light domain form a second antigen binding domain, wherein:
(i) the first monomer and the second monomer comprise a first set of VH:VL electrostatic variants comprising amino acid substitutions Q39E:Q38K, and a set of CH1:CL electrostatic variants comprising amino acid substitutions K213E/K218D:D122K/E123K; and
(ii) the third monomer and the fourth monomer comprise a second set of VH:VL electrostatic variants comprising amino acid substitutions Q39K:Q38E, and a set of CH1:CL steric variants comprising amino acid substitutions A141F:F118A; and
wherein numbering of the first set and second set of VH:VL electrostatic variants is according to Kabat numbering, and numbering of the set of CH1:CL electrostatic variants and the set of CH1:CL steric variants is according to EU numbering.

153. A multimeric protein, comprising:
(a) a first monomer comprising a VH1-CH1-hinge-CH2-CH3 monomer, wherein VH1 is a first variable heavy domain and CH2-CH3 is a first variant IgG Fc domain;
(b) a second monomer comprising a VL1-CL1 monomer, wherein VL1 is a first variable light domain, and wherein the first variable heavy domain and the first variable light domain form a first antigen binding domain;
(c) a third monomer comprising a VH2-CH1-hinge-CH2-CH3 monomer, wherein VH2 is a second variable heavy domain and CH2-CH3 is a second variant IgG Fc domain; and
(d) a fourth monomer comprising a VL2-CL2 monomer, wherein VL2 is a second variable light domain, and wherein the second variable heavy domain and the second variable light domain form a second antigen binding domain, wherein:
(i) the first monomer and the second monomer comprise a first set of VH:VL electrostatic variants comprising amino acid substitutions Q39E:Q38K, and a set of CH1:CL electrostatic variants comprising amino acid substitutions K213E/K218D:D122K/E123K; and
(ii) the third monomer and the fourth monomer comprise a second set of VH:VL electrostatic variants comprising amino acid substitutions Q39K:Q38E, and a set of CH1:CL steric variants comprising amino acid substitutions K147S:S131K; and
wherein numbering of the first set and second set of VH:VL electrostatic variants is according to Kabat numbering, and numbering of the set of CH1:CL electrostatic variants and the set of CH1:CL steric variants is according to EU numbering.

154. A multimeric protein, comprising:
(a) a first monomer comprising a VH-CH1 domain, wherein VH is a variable heavy domain; and
(b) a second monomer comprising a VL-CL domain, wherein VL is a variable light domain, wherein:
(i) the variable heavy domain and the variable light domain form an antigen binding domain; and
(ii) the first monomer and the second monomer comprise a set of CH1:CL electrostatic variants comprising amino acid substitutions at amino acid residues K213/K218:D122/E123, wherein numbering is according to EU numbering.

155.-161. (canceled)

162. A multimeric protein, comprising:
(a) a first monomer comprising a VH-CH1 domain, wherein VH is a variable heavy domain; and
(b) a second monomer comprising a VL-CL domain, wherein VL is a variable light domain, wherein:
(i) the variable heavy domain and the variable light domain form an antigen binding domain; and
(ii) the first monomer and the second monomer comprise a set of CH1:CL steric variants comprising amino acid substitutions at amino acid residues selected from the group consisting of: (i) A141:F118, (ii) A141:F116, and (iii) K147:S131, wherein numbering is according to EU numbering.

163.-169. (canceled)

170. A multimeric protein, comprising:
(a) a first monomer comprising a VH-CH1 domain, wherein VH is a variable heavy domain; and
(b) a second monomer comprising a VL-CL domain, wherein VL is a variable light domain, wherein:
(i) the variable heavy domain and the variable light domain form an antigen binding domain; and
(ii) the first monomer and the second monomer comprise a set of VH:VL electrostatic variants comprising amino acid substitutions at amino acid residues Q39:Q38, wherein numbering is according to Kabat numbering.

171.-177. (canceled)

178. A multimeric protein, comprising:
(a) a variant human IgG1 CH1 domain (vCH1); and
(b) a variant human kappa CL domain (vCL),
wherein said CH1 domain and said CL domain together have a set of amino acid substitutions selected from the group consisting of: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R, wherein numbering is according to EU numbering.

179. A multimeric protein, comprising:
(a) a variant human IgG1 CH1 domain (vCH1); and
(b) a variant human kappa CL domain (vCL), wherein:
(1) said CH1 domain and said CL domain together have a first set of amino acid substitutions selected from the group consisting of: (i) K213E/K218D:D122K/E123K, (ii) K213E/K218E:D122K/E123K, (iii) K213D/K218E:D122K/E123K, (iv) K213D/K218D:D122K/E123K, (v) K213E/K218D:D122K/E123R, (vi) K213E/K218E:D122K/E123R, (vii) K213D/K218E:D122K/E123R, (viii) K213D/K218D:D122K/E123R, (ix) K213E/K218D:D122R/E123K, (x) K213E/K218E:D122R/E123K, (xi) K213D/K218E:D122R/E123K, (xii) K213D/K218D:D122R/E123K, (xiii) K213E/K218D:D122R/E123R, (xiv) K213E/K218E:D122R/E123R, (xv) K213D/K218E:D122R/E123R, and (xvi) K213D/K218D:D122R/E123R,
(2) said CH1 domain and said CL domain together have a second set of amino acid substitutions selected from the group consisting of: (i) A141F:F118A, (ii) A141F:F116A, and (iii) K147S:S131K, and
(3) numbering of said first set of amino acid substitutions and numbering of said second set of amino acid substitutions are both according to EU numbering.
